SEQUENCE LISTING (97 sequences)

SEQ ID NO: 1 - protein, 16 aa, Artificial Sequence
  Made in Lab - Anti-CD79A-01 CDRL1
RSSQSIVHSNGNTYLE

SEQ ID NO: 2 - protein, 7 aa, Artificial Sequence
  Made in Lab - Anti-CD79A-01 CDRL2
KVSNRFS

SEQ ID NO: 3 - protein, 9 aa, Artificial Sequence
  Made in Lab - Anti-CD79A-01 CDRL3
FQGSHVPFT

SEQ ID NO: 4 - protein, 10 aa, Artificial Sequence
  Made in Lab - Anti-CD79A-01 CDRH1
GYTFSTSWMN

SEQ ID NO: 5 - protein, 17 aa, Artificial Sequence
  Made in Lab - Anti-CD79A-01 CDRH2
RIYPGDGDTNYNGKFKG

SEQ ID NO: 6 - protein, 11 aa, Artificial Sequence
  Made in Lab - Anti-CD79A-01 CDRH3
FYYGNTFAMDY

SEQ ID NO: 7 - protein, 112 aa, Artificial Seqauence
  Made in Lab - anti-CD79A-01 Light chain
DVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 8 - protein, 120 aa, Artificial Seqauence
  Made in Lab - anti-CD79A-01 Heavy chain
QVQLQQSGPELVKPGASVKISCKASGYTFSTSWMNWVKQRPGQGLEWIGRIYPGDGDTNYNGKFKGKATLTADKSSNTAYMQLSSLTSVDSAVYFCERFYYGNTFAMDYWGQGTSVTVSS

SEQ ID NO: 9 - protein, 16 aa, Artificial Sequence
  Made in Lab - Anti-CD79A-10 CDRL1
RSSQSLVHSNGNTYLH

SEQ ID NO: 10 - protein, 7 aa, Artificial Sequence
  Made in Lab - Anti-CD79A-10 CDRL2
KVSNRFS

SEQ ID NO: 11 - protein, 10 aa, Artificial Sequence
  Made in Lab - Anti-CD79A-10 CDRL3
SQSTHVPPYT

SEQ ID NO: 12 - protein, 10 aa, Artificial Sequence
  Made in Lab - Anti-CD79A-19 CDRH1
GYAFSFSWMN

SEQ ID NO: 13 - protein, 17 aa, Artificial Sequence
  Made in Lab - Anti-CD79A-10 CDRH2
RIYPGNGDTNYNGKFKG

SEQ ID NO: 14 - protein, 12 aa, Artificial Sequence
  Made in Lab - Anti-CD79A-10 CDRH3
WVYSGNNYAVDY

SEQ ID NO: 15 - protein, 113 aa, Artificial Seqauence
  Made in Lab - anti-CD79A-10 Light chain
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPYTFGGGTKLEIK

SEQ ID NO: 16 - protein, 121 aa, Artificial Seqauence
  Made in Lab - anti-CD79A-10 Heavy chain
QVQLQQSGPELVKPGASVKISCKTSGYAFSFSWMNWVKWGPGQGLEWIGRIYPGNGDTNYNGKFKGKATLTADKSSNTAYMQLSSLTSVDSAVYFCARWVYSGNNYAVDYWGQGTSVTVSS

SEQ ID NO: 17 - protein, 492 aa, Artificial Sequence
  Made in Lab - anti-CD79A-01_HL CAR construct
MALPVTALLLPLALLLHAARPQVQLQQSGPELVKPGASVKISCKASGYTFSTSWMNWVKQRPGQGLEWIGRIYPGDGDTNYNGKFKGKATLTADKSSNTAYMQLSSLTSVDSAVYFCERFYYGNTFAMDYWGQGTSVTVSSGGGGSGGGGSGGGGSDVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 492 aa, Artificial Sequence
  Made in Lab - anti-CD79A-01_LH CAR construct
MALPVTALLLPLALLLHAARPDVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRGGGGSGGGGSGGGGSQVQLQQSGPELVKPGASVKISCKASGYTFSTSWMNWVKQRPGQGLEWIGRIYPGDGDTNYNGKFKGKATLTADKSSNTAYMQLSSLTSVDSAVYFCERFYYGNTFAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 493 aa, Artificial Sequence
  Made in Lab - anti-CD79A-10_HL CAR construct
MALPVTALLLPLALLLHAARPQVQLQQSGPELVKPGASVKISCKTSGYAFSFSWMNWVKWGPGQGLEWIGRIYPGNGDTNYNGKFKGKATLTADKSSNTAYMQLSSLTSVDSAVYFCARWVYSGNNYAVDYWGQGTSVTVSSGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPYTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 493 aa, Artificial Sequence
  Made in Lab - anti-CD79A-10_LH CAR construct
MALPVTALLLPLALLLHAARPDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPYTFGGGTKLEIKGGGGSGGGGSGGGGSQVQLQQSGPELVKPGASVKISCKTSGYAFSFSWMNWVKWGPGQGLEWIGRIYPGNGDTNYNGKFKGKATLTADKSSNTAYMQLSSLTSVDSAVYFCARWVYSGNNYAVDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 1479 bp, Artificial Sequence
  Made in Lab - anti-CD79A-01_HL CAR polynucleotide construct
atggcacttccagtgactgcattgttgttgcccctggccctgctgcttcacgccgcaaggccccaggtccagcttcagcagtccggaccagagttggtgaaacctggggcatcagtcaagattagctgcaaggcatcagggtacaccttcagcactagttggatgaattgggtgaagcagcggcctggccaggggttggagtggatcggacgcatctacccaggggatggcgatacaaactacaacggaaagtttaaaggtaaggctactttgaccgctgacaaatcatcaaacacggcatatatgcaactgtctagcctcacatctgtcgactctgccgtctacttttgcgagcgattctactatggcaacacttttgccatggactactgggggcaggggacatcagtaacagttagttctggagggggcggttccgggggcgggggatccggagggggaggcagtgatgtgctgatgacccagataccgttgtcactcccggtctcactgggtgaccaggcaagcatcagctgcaggagcagccagtccatcgtgcactctaacggaaatacctacctggagtggtatctgcagaaacctggccagtcccctaagttgttgatttacaaagtttcaaaccgatttagtggagttcccgataggttttccggttccgggagcggtacagacttcaccctgaagataagtcgggttgaggctgaagacctgggagtatattattgctttcagggcagccacgtaccattcacatttgggagtggaaccaaactggagatcaaaagaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaa

SEQ ID NO: 22 - DNA, 1479 bp, Artificial Sequence
  Made in Lab - anti-CD79A-01_LH CAR polynucleotide construct
atggctctgcctgtgacggccctgcttttgcccctcgccctgcttctgcatgccgcgagacccgacgtgttgatgacccaaataccgcttagtctgcctgtatctcttggggaccaggctagcatctcatgccgcagcagtcaatccattgtgcactcaaatgggaacacctatttggagtggtatctgcaaaaaccgggacagtctccgaaactgctgatatacaaagtaagcaacaggttcagcggagttcctgacagattcagcggaagcggttctggaactgactttacacttaagatctctcgcgttgaggcggaggacctgggcgtgtattactgttttcaaggatcccacgtcccgtttacattcggatcaggcaccaagctggagatcaagcgcggtggcgggggttctggcgggggcggatccggaggcggcggatcccaggtgcagctgcagcagtctggaccagaactggttaagcccggagcttcagttaagatttcctgtaaggcttcaggctatacattttccacttcttggatgaactgggtgaaacagcgccctggccaggggctggaatggatcggacggatctatcccggcgatggagacactaattataacggtaagttcaaagggaaggccaccctcacggccgacaagtcctccaatacagcgtacatgcaactcagttccctgaccagcgttgatagcgcagtttacttctgtgagcgcttctattacggaaacaccttcgctatggattactggggtcaggggacctccgtgaccgtgtcctctaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaa

SEQ ID NO: 23 - DNA, 1482 bp, Artificial Sequence
  Made in Lab - anti-CD79A-10_HL CAR polynucleotide construct
atggctctgccagtgactgcgctgctgctgcccctcgctcttctgctgcacgccgctcggccacaggtccaactgcaacagagcggccccgagctggtaaaacccggggcctccgtaaaaatatcctgcaagaccagcggctatgccttttcattctcctggatgaactgggtgaagtggggacccggtcagggacttgagtggatcgggcgaatctatcccggaaacggggacacgaattacaacggcaaatttaaaggcaaggctactctgactgctgacaaaagtagcaacaccgcctacatgcagttgtcctctttgacatcagtagactctgcagtgtatttttgcgcccggtgggtttactccggaaataactacgcggttgactattggggacagggcacctccgtgacagtgtcttctggcggcgggggatcaggtggcggcgggtctgggggtggagggagcgacgtggttatgacccagacccctttgagcctgcccgtgagccttggcgaccaagcctccatctcttgccggtcctctcaatcactggtgcacagtaacgggaatacttatctccactggtatttgcaaaagcccggtcagtctcctaagcttctgatctataaggtgtccaaccgcttttctggagtgcccgatagattttccggatctggatccggaacagacttcacattgaagattagtagagtcgaagcggaggacttgggtgtttatttttgttcccagagtacccacgtgcctccatacacgtttggaggtggaaccaaacttgaaattaagaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaa

SEQ ID NO: 24 - DNA, 1482 bp, Artificial Sequence
  Made in Lab - anti-CD79A-10_LH CAR polynucleotide construct
atggccctgccagtaacagcactgctccttcctctcgcactcctcctgcacgcagcaagaccagatgtcgtgatgactcagacacctcttagcttgcccgtctccttgggtgatcaggctagcatttcatgtagatcaagccagagcttggtacattcaaatggcaatacctacctgcactggtatctccagaaacctggccagagcccaaaattgcttatctataaagttagtaacagattctccggggtcccagatcgattttccggctcaggctcaggcacggattttaccctgaagatctcacgagtcgaggcagaagatcttggcgtgtacttttgttcccagagtacccacgtacccccttacacattcggaggagggaccaagttggagatcaaaggaggggggggcagtggggggggcggttccggagggggaggaagccaagtacaactgcaacaaagcggacccgagctcgtcaaacctggtgctagcgtcaaaatttcctgtaagaccagcggctacgcttttagcttttcttggatgaactgggtgaaatgggggccggggcagggcctggagtggataggacgaatttatcccgggaatggggatacaaattacaatggtaaattcaaggggaaagccacactgactgcagataaaagctccaacacggcatacatgcagctcagctccctcacttctgtggattctgctgtttacttttgtgccaggtgggtttactctggtaataactatgccgtggattactgggggcaaggcacctctgttacagtgagttccaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaa

SEQ ID NO: 25 - protein, 10 aa, Artificial Sequence
  Made in Lab - Anti-CD20 CDRL1
RASSSVNYMD

SEQ ID NO: 26 - protein, 7 aa, Artificial Sequence
  Made in Lab - Anti-CD20 CDRL2
ATSNLAS

SEQ ID NO: 27 - protein, 9 aa, Artificial Sequence
  Made in Lab - Anti-CD20 CDRL3
QQWSFNPPT

SEQ ID NO: 28 - protein, 10 aa, Artificial Sequence
  Made in Lab - Anti-CD20 CDRH1
GYTFTSYNMH

SEQ ID NO: 29 - protein, 17 aa, Artificial Sequence
  Made in Lab - Anti-CD20 CDRH2
AIYPGNGDTSYNQKFKG

SEQ ID NO: 30 - protein, 13 aa, Artificial Sequence
  Made in Lab - Anti-CD20 CDRH3
SNYYGSSYWFFDV

SEQ ID NO: 31 - protein, 106 aa, Artificial Seqauence
  Made in Lab - anti-CD20 Light chain
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 32 - protein, 122 aa, Artificial Seqauence
  Made in Lab - anti-CD20 Heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 33 - protein, 374 aa, Artificial Sequence
  Made in Lab - anti-CD20 L-H CCR construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 34 - DNA, 1125 bp, Artificial Sequence
  Made in Lab - anti-CD20 L-H CCR polynucleotide construct
atggcgctccctgtcacagcactgctccttccgctggctctgcttctgcacgctgctaggccagaagttcaactccagcagagcggcgcagagctcgtgaagcccggagcttcagtgaagatgtcatgtaaagcctcaggatatacttttacttcatacaatatgcactgggtgaagcagacaccgggtcagggacttgagtggatcggcgcaatataccccggaaatggagacaccagctacaaccagaaatttaagggaaaagccaccctgacagccgataaatcctccagtacggcttacatgcaattgagctcactgactagcgaggactccgcagattattattgtgctagaagtaactactacggcagctcctattggttttttgacgtgtggggggcgggcaccaccgttacagtcagcagtggcggcggaggttcaggtggggggggctctggaggcggtgggtctgatatcgtcttgactcaaagcccagcgatattgtcagcctcccctggagagaaagtgactatgacctgcagggctagcagctctgttaattatatggattggtatcagaagaaacctggcagctcccctaagccctggatttatgctacatcaaatctcgcctcaggggtgccagccaggttcagtggatccggcagtggcaccagctatagcctgacaatctcaagggtcgaagcagaggacgccgcaacatattattgtcagcaatggagctttaatccccccacatttggcggaggaaccaagttggagatcaagaccacgaccccggcaccccggccacctacaccagcccctacaattgctagccagcccctgagccttaggccagaagcctgtagacccgccgccggcggtgcggttcacacccggggactcgacttcgcctgtgatatatatatctgggcacccctggccggcacatgtggagtgcttctgctgtccttggtcattaccctctactgcagatctaaaagatccagactgcttcattctgactatatgaatatgactcctagacggcctgggcccaccaggaagcactaccagccatacgccccaccacgagattttgccgcttatcggtcctaa

SEQ ID NO: 35 - protein, 374 aa, Artificial Sequence
  Made in Lab - anti-CD20 H-L CCR construct
MALPVTALLLPLALLLHAARPDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 36 - DNA, 1125 bp, Artificial Sequence
  Made in Lab - anti-CD20 H-L CCR polynucleotide construct
atggcgctccctgtcacagcactgctccttccgctggctctgcttctgcacgctgctaggccagatatcgtcttgactcaaagcccagcgatattgtcagcctcccctggagagaaagtgactatgacctgcagggctagcagctctgttaattatatggattggtatcagaagaaacctggcagctcccctaagccctggatttatgctacatcaaatctcgcctcaggggtgccagccaggttcagtggatccggcagtggcaccagctatagcctgacaatctcaagggtcgaagcagaggacgccgcaacatattattgtcagcaatggagctttaatccccccacatttggcggaggaaccaagttggagatcaagggcggcggaggttcaggtggggggggctctggaggcggtgggtctgaagttcaactccagcagagcggcgcagagctcgtgaagcccggagcttcagtgaagatgtcatgtaaagcctcaggatatacttttacttcatacaatatgcactgggtgaagcagacaccgggtcagggacttgagtggatcggcgcaatataccccggaaatggagacaccagctacaaccagaaatttaagggaaaagccaccctgacagccgataaatcctccagtacggcttacatgcaattgagctcactgactagcgaggactccgcagattattattgtgctagaagtaactactacggcagctcctattggttttttgacgtgtggggggcgggcaccaccgttacagtcagcagtaccacgaccccggcaccccggccacctacaccagcccctacaattgctagccagcccctgagccttaggccagaagcctgtagacccgccgccggcggtgcggttcacacccggggactcgacttcgcctgtgatatatatatctgggcacccctggccggcacatgtggagtgcttctgctgtccttggtcattaccctctactgcagatctaaaagatccagactgcttcattctgactatatgaatatgactcctagacggcctgggcccaccaggaagcactaccagccatacgccccaccacgagattttgccgcttatcggtcctaa

SEQ ID NO: 37 - protein, 887 aa, Artificial Sequence
  Made in Lab - anti-CD79A-01_LH CAR - T2A - anti-CD20 CCR construct
MALPVTALLLPLALLLHAARPDVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRGGGGSGGGGSGGGGSQVQLQQSGPELVKPGASVKISCKASGYTFSTSWMNWVKQRPGQGLEWIGRIYPGDGDTNYNGKFKGKATLTADKSSNTAYMQLSSLTSVDSAVYFCERFYYGNTFAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 38 - DNA, 2664 bp, Artificial Sequence
  Made in Lab - anti-CD79A-01_LH CAR - T2A - CD20 CCR polynucleotide construct
atggctctgcctgtgacggccctgcttttgcccctcgccctgcttctgcatgccgcgagacccgacgtgttgatgacccaaataccgcttagtctgcctgtatctcttggggaccaggctagcatctcatgccgcagcagtcaatccattgtgcactcaaatgggaacacctatttggagtggtatctgcaaaaaccgggacagtctccgaaactgctgatatacaaagtaagcaacaggttcagcggagttcctgacagattcagcggaagcggttctggaactgactttacacttaagatctctcgcgttgaggcggaggacctgggcgtgtattactgttttcaaggatcccacgtcccgtttacattcggatcaggcaccaagctggagatcaagcgcggtggcgggggttctggcgggggcggatccggaggcggcggatcccaggtgcagctgcagcagtctggaccagaactggttaagcccggagcttcagttaagatttcctgtaaggcttcaggctatacattttccacttcttggatgaactgggtgaaacagcgccctggccaggggctggaatggatcggacggatctatcccggcgatggagacactaattataacggtaagttcaaagggaaggccaccctcacggccgacaagtcctccaatacagcgtacatgcaactcagttccctgaccagcgttgatagcgcagtttacttctgtgagcgcttctattacggaaacaccttcgctatggattactggggtcaggggacctccgtgaccgtgtcctctaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcgggtccggtgagggacgaggatctctgttaacgtgtggcgatgtcgaggaaaatcctggcccaatggcgctccctgtcacagcactgctccttccgctggctctgcttctgcacgctgctaggccagaagttcaactccagcagagcggcgcagagctcgtgaagcccggagcttcagtgaagatgtcatgtaaagcctcaggatatacttttacttcatacaatatgcactgggtgaagcagacaccgggtcagggacttgagtggatcggcgcaatataccccggaaatggagacaccagctacaaccagaaatttaagggaaaagccaccctgacagccgataaatcctccagtacggcttacatgcaattgagctcactgactagcgaggactccgcagattattattgtgctagaagtaactactacggcagctcctattggttttttgacgtgtggggggcgggcaccaccgttacagtcagcagtggcggcggaggttcaggtggggggggctctggaggcggtgggtctgatatcgtcttgactcaaagcccagcgatattgtcagcctcccctggagagaaagtgactatgacctgcagggctagcagctctgttaattatatggattggtatcagaagaaacctggcagctcccctaagccctggatttatgctacatcaaatctcgcctcaggggtgccagccaggttcagtggatccggcagtggcaccagctatagcctgacaatctcaagggtcgaagcagaggacgccgcaacatattattgtcagcaatggagctttaatccccccacatttggcggaggaaccaagttggagatcaagaccacgaccccggcaccccggccacctacaccagcccctacaattgctagccagcccctgagccttaggccagaagcctgtagacccgccgccggcggtgcggttcacacccggggactcgacttcgcctgtgatatatatatctgggcacccctggccggcacatgtggagtgcttctgctgtccttggtcattaccctctactgcagatctaaaagatccagactgcttcattctgactatatgaatatgactcctagacggcctgggcccaccaggaagcactaccagccatacgccccaccacgagattttgccgcttatcggtcctaa

SEQ ID NO: 39 - protein, 888 aa, Artificial Sequence
  Made in Lab - anti-CD79A-10_HL CAR - T2A - anti-CD20 CCR construct
MALPVTALLLPLALLLHAARPQVQLQQSGPELVKPGASVKISCKTSGYAFSFSWMNWVKWGPGQGLEWIGRIYPGNGDTNYNGKFKGKATLTADKSSNTAYMQLSSLTSVDSAVYFCARWVYSGNNYAVDYWGQGTSVTVSSGGGGSGGGGSGGGRSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPYTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 40 - DNA, 2667 bp, Artificial Sequence
  Made in Lab - anti-CD79A-10_HL CAR - T2A - anti-CD20 CCR polynucleotide construct
atggctctgccagtgactgcgctgctgctgcccctcgctcttctgctgcacgccgctcggccacaggtccaactgcaacagagcggccccgagctggtaaaacccggggcctccgtaaaaatatcctgcaagaccagcggctatgccttttcattctcctggatgaactgggtgaagtggggacccggtcagggacttgagtggatcgggcgaatctatcccggaaacggggacacgaattacaacggcaaatttaaaggcaaggctactctgactgctgacaaaagtagcaacaccgcctacatgcagttgtcctctttgacatcagtagactctgcagtgtatttttgcgcccggtgggtttactccggaaataactacgcggttgactattggggacagggcacctccgtgacagtgtcttctggcggcgggggatcaggtggcggcgggtctgggggtggaaggagcgacgtggttatgacccagacccctttgagcctgcccgtgagccttggcgaccaagcctccatctcttgccggtcctctcaatcactggtgcacagtaacgggaatacttatctccactggtatttgcaaaagcccggtcagtctcctaagcttctgatctataaggtgtccaaccgcttttctggagtgcccgatagattttccggatctggatccgggacagacttcacattgaagattagtagagtcgaagcggaggacttgggtgtttatttttgttcccagagtacccacgtgcctccatacacgtttggaggtggaaccaaacttgaaattaagaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcgggtccggtgagggacgaggatctctgttaacgtgtggcgatgtcgaggaaaatcctggcccaatggcgctccctgtcacagcactgctccttccgctggctctgcttctgcacgctgctaggccagaagttcaactccagcagagcggcgcagagctcgtgaagcccggagcttcagtgaagatgtcatgtaaagcctcaggatatacttttacttcatacaatatgcactgggtgaagcagacaccgggtcagggacttgagtggatcggcgcaatataccccggaaatggagacaccagctacaaccagaaatttaagggaaaagccaccctgacagccgataaatcctccagtacggcttacatgcaattgagctcactgactagcgaggactccgcagattattattgtgctagaagtaactactacggcagctcctattggttttttgacgtgtggggggcgggcaccaccgttacagtcagcagtggcggcggaggttcaggtggggggggctctggaggcggtgggtctgatatcgtcttgactcaaagcccagcgatattgtcagcctcccctggagagaaagtgactatgacctgcagggctagcagctctgttaattatatggattggtatcagaagaaacctggcagctcccctaagccctggatttatgctacatcaaatctcgcctcaggggtgccagccaggttcagtggatccggcagtggcaccagctatagcctgacaatctcaagggtcgaagcagaggacgccgcaacatattattgtcagcaatggagctttaatccccccacatttggcggaggaaccaagttggagatcaagaccacgaccccggcaccccggccacctacaccagcccctacaattgctagccagcccctgagccttaggccagaagcctgtagacccgccgccggcggtgcggttcacacccggggactcgacttcgcctgtgatatatatatctgggcacccctggccggcacatgtggagtgcttctgctgtccttggtcattaccctctactgcagatctaaaagatccagactgcttcattctgactatatgaatatgactcctagacggcctgggcccaccaggaagcactaccagccatacgccccaccacgagattttgccgcttatcggtcctaa

SEQ ID NO: 41 - protein, 999 aa, Artificial Sequence
  Made in Lab - anti-CD79A-01_LH CAR-T2A-anti- CD20 CD28z CAR construct
MALPVTALLLPLALLLHAARPDVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRGGGGSGGGGSGGGGSQVQLQQSGPELVKPGASVKISCKASGYTFSTSWMNWVKQRPGQGLEWIGRIYPGDGDTNYNGKFKGKATLTADKSSNTAYMQLSSLTSVDSAVYFCERFYYGNTFAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 3000 bp, Artificial Sequence
  Made in Lab - anti-CD79A-01_LH CAR-T2A-anti- CD20 CD28z CAR polynucleotide construct
atggctctgcctgtgacggccctgcttttgcccctcgccctgcttctgcatgccgcgagacccgacgtgttgatgacccaaataccgcttagtctgcctgtatctcttggggaccaggctagcatctcatgccgcagcagtcaatccattgtgcactcaaatgggaacacctatttggagtggtatctgcaaaaaccgggacagtctccgaaactgctgatatacaaagtaagcaacaggttcagcggagttcctgacagattcagcggaagcggttctggaactgactttacacttaagatctctcgcgttgaggcggaggacctgggcgtgtattactgttttcaaggatcccacgtcccgtttacattcggatcaggcaccaagctggagatcaagcgcggtggcgggggttctggcgggggcggatccggaggcggcggatcccaggtgcagctgcagcagtctggaccagaactggttaagcccggagcttcagttaagatttcctgtaaggcttcaggctatacattttccacttcttggatgaactgggtgaaacagcgccctggccaggggctggaatggatcggacggatctatcccggcgatggagacactaattataacggtaagttcaaagggaaggccaccctcacggccgacaagtcctccaatacagcgtacatgcaactcagttccctgaccagcgttgatagcgcagtttacttctgtgagcgcttctattacggaaacaccttcgctatggattactggggtcaggggacctccgtgaccgtgtcctctaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcgggtccggtgagggacgaggatctctgttaacgtgtggcgatgtcgaggaaaatcctggcccaatggcgctccctgtcacagcactgctccttccgctggctctgcttctgcacgctgctaggccagaagttcaactccagcagagcggcgcagagctcgtgaagcccggagcttcagtgaagatgtcatgtaaagcctcaggatatacttttacttcatacaatatgcactgggtgaagcagacaccgggtcagggacttgagtggatcggcgcaatataccccggaaatggagacaccagctacaaccagaaatttaagggaaaagccaccctgacagccgataaatcctccagtacggcttacatgcaattgagctcactgactagcgaggactccgcagattattattgtgctagaagtaactactacggcagctcctattggttttttgacgtgtggggggcgggcaccaccgttacagtcagcagtggcggcggaggttcaggtggggggggctctggaggcggtgggtctgatatcgtcttgactcaaagcccagcgatattgtcagcctcccctggagagaaagtgactatgacctgcagggctagcagctctgttaattatatggattggtatcagaagaaacctggcagctcccctaagccctggatttatgctacatcaaatctcgcctcaggggtgccagccaggttcagtggatccggcagtggcaccagctatagcctgacaatctcaagggtcgaagcagaggacgccgcaacatattattgtcagcaatggagctttaatccccccacatttggcggaggaaccaagttggagatcaagaccacgaccccggcaccccggccacctacaccagcccctacaattgctagccagcccctgagccttaggccagaagcctgtagacccgccgccggcggtgcggttcacacccggggactcgacttcgcctgtgatatatatatctgggcacccctggccggcacatgtggagtgcttctgctgtccttggtcattaccctctactgcagatctaaaagatccagactgcttcattctgactatatgaatatgactcctagacggcctgggcccaccaggaagcactaccagccatacgccccaccacgagattttgccgcttatcggtcccgggttaaattttctaggagcgccgacgcgccagcataccagcagggccagaaccagctctacaacgagctcaacctggggcgcagggaggagtatgacgtgcttgacaagaggagaggccgggatccagagatgggcgggaagcctaggcggaaaaacccacaggaggggttgtacaacgagcttcaaaaggacaaaatggccgaagcctactctgaaataggcatgaagggcgaacgcagacgaggcaaaggccacgacggactgtatcaaggattgtctaccgctacaaaggatacctacgacgcactgcatatgcaggccctgcctcctaggtaa

SEQ ID NO: 43 - protein, 1000 aa, Artificial Sequence
  Made in Lab - anti-CD79A-10_HL CAR-T2A-anti- CD20 CD28z CAR construct
MALPVTALLLPLALLLHAARPQVQLQQSGPELVKPGASVKISCKTSGYAFSFSWMNWVKWGPGQGLEWIGRIYPGNGDTNYNGKFKGKATLTADKSSNTAYMQLSSLTSVDSAVYFCARWVYSGNNYAVDYWGQGTSVTVSSGGGGSGGGGSGGGRSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPYTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 3003 bp, Artificial Sequence
  Made in Lab - anti-CD79A-10_HL CAR-T2A-anti- CD20 CD28z CAR polynucleotide construct
atggctctgccagtgactgcgctgctgctgcccctcgctcttctgctgcacgccgctcggccacaggtccaactgcaacagagcggccccgagctggtaaaacccggggcctccgtaaaaatatcctgcaagaccagcggctatgccttttcattctcctggatgaactgggtgaagtggggacccggtcagggacttgagtggatcgggcgaatctatcccggaaacggggacacgaattacaacggcaaatttaaaggcaaggctactctgactgctgacaaaagtagcaacaccgcctacatgcagttgtcctctttgacatcagtagactctgcagtgtatttttgcgcccggtgggtttactccggaaataactacgcggttgactattggggacagggcacctccgtgacagtgtcttctggcggcgggggatcaggtggcggcgggtctgggggtggaaggagcgacgtggttatgacccagacccctttgagcctgcccgtgagccttggcgaccaagcctccatctcttgccggtcctctcaatcactggtgcacagtaacgggaatacttatctccactggtatttgcaaaagcccggtcagtctcctaagcttctgatctataaggtgtccaaccgcttttctggagtgcccgatagattttccggatctggatccgggacagacttcacattgaagattagtagagtcgaagcggaggacttgggtgtttatttttgttcccagagtacccacgtgcctccatacacgtttggaggtggaaccaaacttgaaattaagaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcgggtccggtgagggacgaggatctctgttaacgtgtggcgatgtcgaggaaaatcctggcccaatggcgctccctgtcacagcactgctccttccgctggctctgcttctgcacgctgctaggccagaagttcaactccagcagagcggcgcagagctcgtgaagcccggagcttcagtgaagatgtcatgtaaagcctcaggatatacttttacttcatacaatatgcactgggtgaagcagacaccgggtcagggacttgagtggatcggcgcaatataccccggaaatggagacaccagctacaaccagaaatttaagggaaaagccaccctgacagccgataaatcctccagtacggcttacatgcaattgagctcactgactagcgaggactccgcagattattattgtgctagaagtaactactacggcagctcctattggttttttgacgtgtggggggcgggcaccaccgttacagtcagcagtggcggcggaggttcaggtggggggggctctggaggcggtgggtctgatatcgtcttgactcaaagcccagcgatattgtcagcctcccctggagagaaagtgactatgacctgcagggctagcagctctgttaattatatggattggtatcagaagaaacctggcagctcccctaagccctggatttatgctacatcaaatctcgcctcaggggtgccagccaggttcagtggatccggcagtggcaccagctatagcctgacaatctcaagggtcgaagcagaggacgccgcaacatattattgtcagcaatggagctttaatccccccacatttggcggaggaaccaagttggagatcaagaccacgaccccggcaccccggccacctacaccagcccctacaattgctagccagcccctgagccttaggccagaagcctgtagacccgccgccggcggtgcggttcacacccggggactcgacttcgcctgtgatatatatatctgggcacccctggccggcacatgtggagtgcttctgctgtccttggtcattaccctctactgcagatctaaaagatccagactgcttcattctgactatatgaatatgactcctagacggcctgggcccaccaggaagcactaccagccatacgccccaccacgagattttgccgcttatcggtcccgggttaaattttctaggagcgccgacgcgccagcataccagcagggccagaaccagctctacaacgagctcaacctggggcgcagggaggagtatgacgtgcttgacaagaggagaggccgggatccagagatgggcgggaagcctaggcggaaaaacccacaggaggggttgtacaacgagcttcaaaaggacaaaatggccgaagcctactctgaaataggcatgaagggcgaacgcagacgaggcaaaggccacgacggactgtatcaaggattgtctaccgctacaaaggatacctacgacgcactgcatatgcaggccctgcctcctaggtaa

SEQ ID NO: 45 - protein, 1000 aa, Artificial Sequence
  Made in Lab - anti-CD79A-01_LH CAR-T2A-anti- CD20 BBz CAR construct
MALPVTALLLPLALLLHAARPDVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRGGGGSGGGGSGGGGSQVQLQQSGPELVKPGASVKISCKASGYTFSTSWMNWVKQRPGQGLEWIGRIYPGDGDTNYNGKFKGKATLTADKSSNTAYMQLSSLTSVDSAVYFCERFYYGNTFAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 3003 bp, Artificial Sequene
  Made in Lab - anti-CD79A-01_LH CAR-T2A-anti- CD20 BBz CAR polynucleotide construct
atggctctgcctgtgacggccctgcttttgcccctcgccctgcttctgcatgccgcgagacccgacgtgttgatgacccaaataccgcttagtctgcctgtatctcttggggaccaggctagcatctcatgccgcagcagtcaatccattgtgcactcaaatgggaacacctatttggagtggtatctgcaaaaaccgggacagtctccgaaactgctgatatacaaagtaagcaacaggttcagcggagttcctgacagattcagcggaagcggttctggaactgactttacacttaagatctctcgcgttgaggcggaggacctgggcgtgtattactgttttcaaggatcccacgtcccgtttacattcggatcaggcaccaagctggagatcaagcgcggtggcgggggttctggcgggggcggatccggaggcggcggatcccaggtgcagctgcagcagtctggaccagaactggttaagcccggagcttcagttaagatttcctgtaaggcttcaggctatacattttccacttcttggatgaactgggtgaaacagcgccctggccaggggctggaatggatcggacggatctatcccggcgatggagacactaattataacggtaagttcaaagggaaggccaccctcacggccgacaagtcctccaatacagcgtacatgcaactcagttccctgaccagcgttgatagcgcagtttacttctgtgagcgcttctattacggaaacaccttcgctatggattactggggtcaggggacctccgtgaccgtgtcctctaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcgggtccggtgagggacgaggatctctgttaacgtgtggcgatgtcgaggaaaatcctggcccaatggcgctccctgtcacagcactgctccttccgctggctctgcttctgcacgctgctaggccagaagttcaactccagcagagcggcgcagagctcgtgaagcccggagcttcagtgaagatgtcatgtaaagcctcaggatatacttttacttcatacaatatgcactgggtgaagcagacaccgggtcagggacttgagtggatcggcgcaatataccccggaaatggagacaccagctacaaccagaaatttaagggaaaagccaccctgacagccgataaatcctccagtacggcttacatgcaattgagctcactgactagcgaggactccgcagattattattgtgctagaagtaactactacggcagctcctattggttttttgacgtgtggggggcgggcaccaccgttacagtcagcagtggcggcggaggttcaggtggggggggctctggaggcggtgggtctgatatcgtcttgactcaaagcccagcgatattgtcagcctcccctggagagaaagtgactatgacctgcagggctagcagctctgttaattatatggattggtatcagaagaaacctggcagctcccctaagccctggatttatgctacatcaaatctcgcctcaggggtgccagccaggttcagtggatccggcagtggcaccagctatagcctgacaatctcaagggtcgaagcagaggacgccgcaacatattattgtcagcaatggagctttaatccccccacatttggcggaggaaccaagttggagatcaagaccacgaccccggcaccccggccacctacaccagcccctacaattgctagccagcccctgagccttaggccagaagcctgtagacccgccgccggcggtgcggttcacacccggggactcgacttcgcctgtgatatatatatctgggcacccctggccggcacatgtggagtgcttctgctgtccttggtcattaccctctactgcaaacgcggtcggaagaagcttctgtacatcttcaaacaacccttcatgaggcctgtgcagacaacacaggaggaggatggctgtagttgcagattccctgaagaagaggaaggtggctgcgagctccgggttaaattttctaggagcgccgacgcgccagcataccagcagggccagaaccagctctacaacgagctcaacctggggcgcagggaggagtatgacgtgcttgacaagaggagaggccgggatccagagatgggcgggaagcctaggcggaaaaacccacaggaggggttgtacaacgagcttcaaaaggacaaaatggccgaagcctactctgaaataggcatgaagggcgaacgcagacgaggcaaaggccacgacggactgtatcaaggattgtctaccgctacaaaggatacctacgacgcactgcatatgcaggccctgcctcctaggtaa

SEQ ID NO: 47 - protein, 1001 aa, Artificial Sequence
  Made in Lab - anti-CD79A-10_HL CAR-T2A-anti- CD20 BBz CAR construct
MALPVTALLLPLALLLHAARPQVQLQQSGPELVKPGASVKISCKTSGYAFSFSWMNWVKWGPGQGLEWIGRIYPGNGDTNYNGKFKGKATLTADKSSNTAYMQLSSLTSVDSAVYFCARWVYSGNNYAVDYWGQGTSVTVSSGGGGSGGGGSGGGRSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPYTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - DNA, 3006 bp, Artificial Sequence
  Made in Lab - anti-CD79A-10_HL CAR-T2A-anti- CD20 BBz CAR polynucleotide construct
atggctctgccagtgactgcgctgctgctgcccctcgctcttctgctgcacgccgctcggccacaggtccaactgcaacagagcggccccgagctggtaaaacccggggcctccgtaaaaatatcctgcaagaccagcggctatgccttttcattctcctggatgaactgggtgaagtggggacccggtcagggacttgagtggatcgggcgaatctatcccggaaacggggacacgaattacaacggcaaatttaaaggcaaggctactctgactgctgacaaaagtagcaacaccgcctacatgcagttgtcctctttgacatcagtagactctgcagtgtatttttgcgcccggtgggtttactccggaaataactacgcggttgactattggggacagggcacctccgtgacagtgtcttctggcggcgggggatcaggtggcggcgggtctgggggtggaaggagcgacgtggttatgacccagacccctttgagcctgcccgtgagccttggcgaccaagcctccatctcttgccggtcctctcaatcactggtgcacagtaacgggaatacttatctccactggtatttgcaaaagcccggtcagtctcctaagcttctgatctataaggtgtccaaccgcttttctggagtgcccgatagattttccggatctggatccgggacagacttcacattgaagattagtagagtcgaagcggaggacttgggtgtttatttttgttcccagagtacccacgtgcctccatacacgtttggaggtggaaccaaacttgaaattaagaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcgggtccggtgagggacgaggatctctgttaacgtgtggcgatgtcgaggaaaatcctggcccaatggcgctccctgtcacagcactgctccttccgctggctctgcttctgcacgctgctaggccagaagttcaactccagcagagcggcgcagagctcgtgaagcccggagcttcagtgaagatgtcatgtaaagcctcaggatatacttttacttcatacaatatgcactgggtgaagcagacaccgggtcagggacttgagtggatcggcgcaatataccccggaaatggagacaccagctacaaccagaaatttaagggaaaagccaccctgacagccgataaatcctccagtacggcttacatgcaattgagctcactgactagcgaggactccgcagattattattgtgctagaagtaactactacggcagctcctattggttttttgacgtgtggggggcgggcaccaccgttacagtcagcagtggcggcggaggttcaggtggggggggctctggaggcggtgggtctgatatcgtcttgactcaaagcccagcgatattgtcagcctcccctggagagaaagtgactatgacctgcagggctagcagctctgttaattatatggattggtatcagaagaaacctggcagctcccctaagccctggatttatgctacatcaaatctcgcctcaggggtgccagccaggttcagtggatccggcagtggcaccagctatagcctgacaatctcaagggtcgaagcagaggacgccgcaacatattattgtcagcaatggagctttaatccccccacatttggcggaggaaccaagttggagatcaagaccacgaccccggcaccccggccacctacaccagcccctacaattgctagccagcccctgagccttaggccagaagcctgtagacccgccgccggcggtgcggttcacacccggggactcgacttcgcctgtgatatatatatctgggcacccctggccggcacatgtggagtgcttctgctgtccttggtcattaccctctactgcaaacgcggtcggaagaagcttctgtacatcttcaaacaacccttcatgaggcctgtgcagacaacacaggaggaggatggctgtagttgcagattccctgaagaagaggaaggtggctgcgagctccgggttaaattttctaggagcgccgacgcgccagcataccagcagggccagaaccagctctacaacgagctcaacctggggcgcagggaggagtatgacgtgcttgacaagaggagaggccgggatccagagatgggcgggaagcctaggcggaaaaacccacaggaggggttgtacaacgagcttcaaaaggacaaaatggccgaagcctactctgaaataggcatgaagggcgaacgcagacgaggcaaaggccacgacggactgtatcaaggattgtctaccgctacaaaggatacctacgacgcactgcatatgcaggccctgcctcctaggtaa

SEQ ID NO: 49 - protein, 303 aa, Artificial Sequence
  Made in Lab - PDCD-1 I-OnuI LHE variant
  MOD_RES (1)..(4): Xaa is any amino acid or absent
  MOD_RES (302)..(303): Xaa is any amino acid or absent
XXXXSRRESINPWTLTGFADAEGSFGLSILNRNRGTARYHTRLSFTIMLHNKDKSILENIQSTWKVGSILNNGDHYVSLVVYAFEDLKVIIDHFEKYPLITQKLGDYKLFKQAFSVMENKEHLKENGIKELVRIKAKMNWGLNDELKKAFPEVISRERPLINKNIPNGKWLAGFTSGDGSFFVRLRKSNVNARVRVQLVFEISQHIRDKNLMNSLITYLGCGHIYEGNKSERSWLQFRVEKFSDINDKIIPVFQENTLIGMKLEDFEDWCKVAKLIEEKKHLTESGLDEIKKIKLNMNKRRXX

SEQ ID NO: 50 - protein, 944 aa, Artificial Sequence
  Made in Lab - PDCD-1 megaTAL construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAISRVGGSSRRESINPWTLTGFADAEGSFGLSILNRNRGTARYHTRLSFTIMLHNKDKSILENIQSTWKVGSILNNGDHYVSLVVYAFEDLKVIIDHFEKYPLITQKLGDYKLFKQAFSVMENKEHLKENGIKELVRIKAKMNWGLNDELKKAFPEVISRERPLINKNIPNGKWLAGFTSGDGSFFVRLRKSNVNARVRVQLVFEISQHIRDKNLMNSLITYLGCGHIYEGNKSERSWLQFRVEKFSDINDKIIPVFQENTLIGMKLEDFEDWCKVAKLIEEKKHLTESGLDEIKKIKLNMNKRR

SEQ ID NO: 51 - DNA, 22 bp, Homo sapiens
ggcatgcagatcccacaggcgc

SEQ ID NO: 52 - DNA, 37 bp, Homo sapiens
ggtggggctgctccaggcatgcagatcccacaggcgc

SEQ ID NO: 53 - protein, 306 aa, Artificial Sequence
  Made in Lab - CBLB I-OnuI LHE variant
  MOD_RES (1)..(4): Xaa is any amino acid or absent
  MOD_RES (302)..(306): Xaa is any amino acid or absent
XXXXSRRESINPWILTGFADAEGCFRLDIRNANDLRAGYRTRLAFEIVLHNKDKSILENIQSTWKVGTIYNAGDNAVRLQVTRFEDLKVIIDHFEKYPLITQKLGDYKLFKQAFSVMENKEHLKENGIKELVRIKAKMNWGLNDELKKAFPENISKERSLINKNIPNLKWLAGFTSGDGSFVVELKKRRSPVKVGVRLRFSITQHIRDKNLMNSLITYLGCGRIVENNKSEHSWLEFIVTKFSDINDKIIPVFQENTLIGVKLEDFEDWCKVAKLIEEKKHLTESGLDEIKKIKLNMNKGRXXXXX

SEQ ID NO: 54 - protein, 915 aa, Artificial Sequence
  Made in Lab - CBLB megaTAL construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGCFRLDIRNANDLRAGYRTRLAFEIVLHNKDKSILENIQSTWKVGTIYNAGDNAVRLQVTRFEDLKVIIDHFEKYPLITQKLGDYKLFKQAFSVMENKEHLKENGIKELVRIKAKMNWGLNDELKKAFPENISKERSLINKNIPNLKWLAGFTSGDGSFVVELKKRRSPVKVGVRLRFSITQHIRDKNLMNSLITYLGCGRIVENNKSEHSWLEFIVTKFSDINDKIIPVFQENTLIGVKLEDFEDWCKVAKLIEEKKHLTESGLDEIKKIKLNMNKGRVFSGR

SEQ ID NO: 55 - DNA, 22 bp, Homo sapiens
ctgtaagatattcccatcccca

SEQ ID NO: 56 - DNA, 39 bp, Homo sapiens
ttgttatgaggtatggtctgtaagatattcccatcccca

SEQ ID NO: 57 - protein, 5 aa, Artificial Sequence
  Exemplary linker sequence
DGGGS

SEQ ID NO: 58 - protein, 5 aa, Artificial Sequence
  Exemplary linker sequence
TGEKP

SEQ ID NO: 59 - protein, 4 aa, Artificial Sequence
  Exemplary linker sequence
GGRR

SEQ ID NO: 60 - protein, 5 aa, Artificial Sequence
  Exemplary linker sequence
GGGGS

SEQ ID NO: 61 - protein, 14 aa, Artificial Sequence
  Exemplary linker sequence
EGKSSGSGSESKVD

SEQ ID NO: 62 - protein, 18 aa, Artificial Sequence
  Exemplary linker sequence
KESGSVSSEQLAQFRSLD

SEQ ID NO: 63 - protein, 8 aa, Artificial Sequence
  Exemplary linker sequence
GGRRGGGS

SEQ ID NO: 64 - protein, 9 aa, Artificial Sequence
  Exemplary linker sequence
LRQRDGERP

SEQ ID NO: 65 - protein, 12 aa, Artificial Sequence
  Exemplary linker sequence
LRQKDGGGSERP

SEQ ID NO: 66 - protein, 16 aa, Artificial Sequence
  Exemplary linker sequence
LRQKDGGGSGGGSERP

SEQ ID NO: 67 - protein, 18 aa, Artificial Sequence
  Exemplary linker sequence
GSTSGSGKPGSGEGSTKG

SEQ ID NO: 68 - protein, 7 aa, Artificial Sequence
  Cleavage sequence by TEV protease
  misc_feature (2)..(3): Xaa is any amino acid
  misc_feature (5)..(5): Xaa is any amino acid
  MISC_FEATURE (7)..(7): Xaa = Gly or Ser
EXXYXQX

SEQ ID NO: 69 - protein, 7 aa, Artificial Sequence
  Cleavage sequence by TEV protease
ENLYFQG

SEQ ID NO: 70 - protein, 7 aa, Artificial Sequence
  Cleavage sequence by TEV protease
ENLYFQS

SEQ ID NO: 71 - protein, 22 aa, Artificial Sequence
  Self-cleaving polypeptide comprising 2A site
GSGATNFSLLKQAGDVEENPGP

SEQ ID NO: 72 - protein, 19 aa, Artificial Sequence
  Self-cleaving polypeptide comprising 2A site
ATNFSLLKQAGDVEENPGP

SEQ ID NO: 73 - protein, 14 aa, Artificial Sequence
  Self-cleaving polypeptide comprising 2A site
LLKQAGDVEENPGP

SEQ ID NO: 74 - protein, 21 aa, Artificial Sequence
  Self-cleaving polypeptide comprising 2A site
GSGEGRGSLLTCGDVEENPGP

SEQ ID NO: 75 - protein, 18 aa, Artificial Sequence
  Self-cleaving polypeptide comprising 2A site
EGRGSLLTCGDVEENPGP

SEQ ID NO: 76 - protein, 13 aa, Artificial Sequence
  Self-cleaving polypeptide comprising 2A site
LLTCGDVEENPGP

SEQ ID NO: 77 - protein, 23 aa, Artificial Sequence
  Self-cleaving polypeptide comprising 2A site
GSGQCTNYALLKLAGDVESNPGP

SEQ ID NO: 78 - protein, 20 aa, Artificial Sequence
  Self-cleaving polypeptide comprising 2A site
QCTNYALLKLAGDVESNPGP

SEQ ID NO: 79 - protein, 14 aa, Artificial Sequence
  Self-cleaving polypeptide comprising 2A site
LLKLAGDVESNPGP

SEQ ID NO: 80 - protein, 25 aa, Artificial Sequence
  Self-cleaving polypeptide comprising 2A site
GSGVKQTLNFDLLKLAGDVESNPGP

SEQ ID NO: 81 - protein, 22 aa, Artificial Sequence
  Self-cleaving polypeptide comprising 2A site
VKQTLNFDLLKLAGDVESNPGP

SEQ ID NO: 82 - protein, 14 aa, Artificial Sequence
  Self-cleaving polypeptide comprising 2A site
LLKLAGDVESNPGP

SEQ ID NO: 83 - protein, 19 aa, Artificial Sequence
  Self-cleaving polypeptide comprising 2A site
LLNFDLLKLAGDVESNPGP

SEQ ID NO: 84 - protein, 19 aa, Artificial Sequence
  Self-cleaving polypeptide comprising 2A site
TLNFDLLKLAGDVESNPGP

SEQ ID NO: 85 - protein, 14 aa, Artificial Sequence
  Self-cleaving polypeptide comprising 2A site
LLKLAGDVESNPGP

SEQ ID NO: 86 - protein, 17 aa, Artificial Sequence
  Self-cleaving polypeptide comprising 2A site
NFDLLKLAGDVESNPGP

SEQ ID NO: 87 - protein, 20 aa, Artificial Sequence
  Self-cleaving polypeptide comprising 2A site
QLLNFDLLKLAGDVESNPGP

SEQ ID NO: 88 - protein, 24 aa, Artificial Sequence
  Self-cleaving polypeptide comprising 2A site
APVKQTLNFDLLKLAGDVESNPGP

SEQ ID NO: 89 - protein, 40 aa, Artificial Sequence
  Self-cleaving polypeptide comprising 2A site
VTELLYRMKRAETYCPRPLLAIHPTEARHKQKIVAPVKQT

SEQ ID NO: 90 - protein, 18 aa, Artificial Sequence
  Self-cleaving polypeptide comprising 2A site
LNFDLLKLAGDVESNPGP

SEQ ID NO: 91 - protein, 40 aa, Artificial Sequence
  Self-cleaving polypeptide comprising 2A site
LLAIHPTEARHKQKIVAPVKQTLNFDLLKLAGDVESNPGP

SEQ ID NO: 92 - protein, 33 aa, Artificial Sequence
  Self-cleaving polypeptide comprising 2A site
EARHKQKIVAPVKQTLNFDLLKLAGDVESNPGP

SEQ ID NO: 93 - DNA, 10 bp, Artificial Sequence
  Consensus Kozak sequence
gccrccatgg

SEQ ID NO: 94 - protein, 4 aa, Artificial Sequence
  Exemplary rule for determining heavy chain CDR-H1motif
  MISC_FEATURE (2)..(4): Xaa is any amino acid
CXXX

SEQ ID NO: 95 - protein, 5 aa, Artificial Sequence
  Exemplary rule for determining heavy chain CDR-H2 motif
LEWIG

SEQ ID NO: 96 - protein, 4 aa, Artificial Sequence
  Exemplary rule for determining heavy chain CDR-H3 motif
  MISC_FEATURE (3)..(3): Xaa is any amino acid
WGXG

SEQ ID NO: 97 - protein, 4 aa, Artificial Sequence
  Exemplary rule for determining light chain CDR-L3 motif
  MISC_FEATURE (3)..(3): Xaa is any amino acid
FGXG

(19) United States
(12) Patent Application Publication
(10) Pub. No.: US 2022/0280567 A1
(43) Pub. Date: Sep. 8, 2022

(54) COMPOSITIONS AND METHODS FOR TREATING CANCER

(71) Applicant: 2seventy bio, Inc., Cambridge, MA (US)

(72) Inventor: KEVIN FRIEDMAN, MELROSE, MA (US)

(73) Assignee: 2seventy bio, Inc., Cambridge, MA (US)

(21) Appl. No.: 17/618,747

(22) PCT Filed: Jun. 11, 2020
(86) PCT No.: PCT/US20/37145

§ 371 Date: Dec. 13, 2021

Related U.S. Application Data
(60) Provisional application No. 62/861,838, filed on Jun. 14, 2019.
(60) Provisional application No. 62/991,314, filed on Mar. 18, 2020.

(51) Int. Cl. A61K 35/17; C07K 16/28; C07K 14/725; C07K 14/705; C07K 14/005; A61P 35/00

(52) U.S. Cl. CPC A61K 35/17; C07K 16/2803; C07K 16/2887; C07K 14/7051; C07K 14/70517; C07K 14/70578; C07K 14/005; A61P 35/00; A61K 38/00

(57) ABSTRACT

The invention provides improved compositions for adoptive cell therapies for cancers that express CD79A and/or CD20.

### CROSS REFERENCE TO RELATED APPLICATIONS

This application claims the benefit under 35 U.S.C. § 119(e) of U.S. Provisional Application No. 62/991,314, filed Mar. 18, 2020, and U.S. Provisional Application No. 62/861,838, filed Jun. 14, 2019, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is BLBD_123_02WO_ST25. The text file is 152 KB, created on Jun. 5, 2020, and is being submitted electronically via EFS-Web, concurrent with the filing of the specification.

### BACKGROUND

### Technical Field

The present invention relates to improved compositions and methods for treating cancer. More particularly, the invention relates to fusion polypeptides encoding anti-CD79A chimeric antigen receptors (CARs) and anti-CD20 chimeric costimulatory receptors (CCRs), genetically modified immune effector cells expressing the same, optionally comprising one or more genome edits, and use of these compositions to effectively treat cancer.

### Description of the Related Art

Cancer is a significant health problem throughout the world. Based on rates from 2008-2010, 40.76% of men and women born today will be diagnosed with some form of cancer at some time during their lifetime. 20.37% of men will develop cancer between their 50th and 70th birthdays compared to 15.30% for women. On Jan. 1, 2010, in the United States there were approximately 13,027,914 men and women alive who had a history of cancer—6,078,974 men and 6,948,940 women. It is estimated that 1,660,290 men and women (854,790 men and 805,500 women) in the United States will be diagnosed with and 580,350 men and women will die of cancer of all sites in 2013. Howlader et al. 2013.

Malignant transformation of B cells leads to cancers including, but not limited to lymphomas, e.g., multiple myeloma and non-Hodgkins' lymphoma. The large majority of patients having B cell malignancies, including non-Hodgkin's lymphoma (NHL) and multiple myeloma (MM), are significant contributors to cancer mortality. The response of B cell malignancies to various forms of treatment is mixed. Traditional methods of treating B cell malignancies, including chemotherapy and radiotherapy, have limited utility due to toxic side effects.

New treatments for relapsed/refractory diffuse large B cell lymphoma (DLBCL) have yet to fulfill the unmet need for treatment of this disease as evidence by several recent setbacks in clinical trials that attempted to improve on standard R-CHOP (rituximab [Rituxan] with cyclophosphamide, doxorubicin, vincristine, and prednisone) therapy. The phase III PHOENIX trial combining ibrutinib (Imbruvica) with R-CHOP failed to meet its primary endpoint of improvement in event-free survival. Results from the CORAL and SCHOLAR-1 trials further evidenced the high unmet need for patients with refractory DLBCL. In each of these studies, the long-term overall survival (OS) rate was just 15% to 20% for patients relapsing within 12 months of stem cell transplant or with refractory disease.

CD19 CAR T cell therapies, Yescarta and Kymriah, have been approved to treat patients that have relapsed/refractory DLBCL, and the CD19 CAR T cell therapy, JCAR017, is en route to approval. One major obstacle that still limits the efficacy of such CAR T cell therapies is relapse of “antigen negative” cancers. For example, although anti-CD19 CAR T cell therapy initially results in modest response rates in relapsed and refractory acute DLBCL, there is the strong possibility of relapse of CD19 negative blasts. Modestly effective CAR T cell therapies combined with the alarmingly high rate of antigen negative relapse reaffirms the unmet medical need of providing highly effective CAR T immunotherapies to DLBCL patients.

### BRIEF SUMMARY

The invention generally provides improved adoptive cell therapies and methods of using the same. More particularly, the invention provides adoptive cell therapies for the prevention, treatment, or amelioration of at least one symptom of cancers that express CD79A and/or CD20.

In various embodiments, a fusion polypeptide is provided comprising an anti-CD79A chimeric antigen receptor (CAR), a polypeptide cleavage signal, and an anti-CD20 chimeric costimulatory receptor (CCR).

In particular embodiments, the anti-CD79A CAR comprises an anti-CD79A antibody or antigen binding fragment thereof; a first transmembrane domain; a first intracellular costimulatory signaling domain; and a primary signaling domain.

In particular embodiments, the anti-CD79A CAR comprises an anti-CD79A antibody or antigen binding fragment thereof selected from the group consisting of: a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, Nanobody).

In various embodiments, the anti-CD79A CAR comprises an anti-CD79A antibody or antigen binding fragment thereof that is an scFv.

In certain embodiments, the anti-CD79A CAR comprises an anti-CD79A antibody or antigen binding fragment thereof comprising a variable light chain sequence comprising CDRL1-CDRL3 sequences set forth in SEQ ID NOs: 1-3 or 9-11 and a variable heavy chain sequence comprising CDRH1-CDRH3 sequences set forth in SEQ ID NOs: 4-6 or 12-14.

In particular embodiments, the anti-CD79A CAR comprises an anti-CD79A antibody or antigen binding fragment thereof comprising the light chain CDRs as set forth in SEQ ID NOs: 1-3 and the heavy chain CDRs as set forth in SEQ ID NOs: 4-6.

In some embodiments, the anti-CD79A CAR comprises an anti-CD79A antibody or antigen binding fragment thereof comprising the light chain CDRs as set forth in SEQ ID NOs: 9-11 and the heavy chain CDRs as set forth in SEQ ID NOs: 12-14.

In various embodiments, the anti-CD79A CAR comprises an anti-CD79A antibody or antigen binding fragment thereof comprising a variable light chain sequence as set forth in any one of SEQ ID NOs: 7 or 15 and a variable heavy chain sequence as set forth in any one of SEQ ID NOs: 8 or 16.

In particular embodiments, the anti-CD79A CAR comprises an anti-CD79A antibody or antigen binding fragment thereof comprising a variable light chain sequence as set forth in SEQ ID NO: 7 and/or a variable heavy chain sequence as set forth in SEQ ID NO: 8.

In certain embodiments, the anti-CD79A CAR comprises an anti-CD79A antibody or antigen binding fragment thereof comprising a variable light chain sequence as set forth in SEQ ID NO: 15 and/or a variable heavy chain sequence as set forth in SEQ ID NO: 16.

In particular embodiments, the anti-CD79A CAR comprises a first transmembrane domain isolated from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1.

In some embodiments, the anti-CD79A CAR comprises a first transmembrane domain isolated from CD8α.

In various embodiments, the anti-CD79A CAR comprises a first costimulatory signaling domain isolated from a costimulatory molecule selected from the group consisting of: TLR1, TLR2, TLR3, TLR4, TLR5, TLR6, TLR7, TLR8, TLR9, TLR10, CARD 11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

In certain embodiments, the anti-CD79A CAR comprises a first costimulatory signaling domain isolated from CD137.

In particular embodiments, the anti-CD79A CAR comprises a primary signaling domain isolated from a polypeptide selected from the group consisting of: FcRγ, FcRβ, CD3γ, CD3δ, CD3ε, CD3ζ, CD22, CD79a, CD79b, and CD66d.

In further embodiments, the anti-CD79A CAR comprises a primary signaling domain isolated from CD3ζ.

In some embodiments, a fusion polypeptide comprising an anti-CD79A CAR comprising a variable light chain sequence as set forth in any one of SEQ ID NOs: 7 or 15 and a variable heavy chain sequence as set forth in any one of SEQ ID NOs: 8 or 16, a CD8α hinge domain, a CD8α transmembrane domain, a CD137 costimulatory domain and a CD3ζ primary signaling domain, a polypeptide cleavage signal, and an anti-CD20 CCR is provided.

In particular embodiments, the polypeptide cleavage signal is a viral self-cleaving polypeptide.

In various embodiments, the polypeptide cleavage signal is a viral self-cleaving 2A polypeptide.

In some embodiments, the polypeptide cleavage signal is a viral self-cleaving polypeptide selected from the group consisting of: a foot-and-mouth disease virus (FMDV) 2A (F2A) peptide, an equine rhinitis A virus (ERAV) 2A (E2A) peptide, a Thosea asigna virus (TaV) 2A (T2A) peptide, a porcine teschovirus-1 (PTV-1) 2A (P2A) peptide, a Theilovirus 2A peptide, and an encephalomyocarditis virus 2A peptide.

In certain embodiments, a fusion polypeptide comprising an anti-CD79A CAR comprising an amino acid sequence as set forth in any one of SEQ ID NOs: 17-20, a T2A self-cleaving polypeptide, and an anti-CD20 CCR contemplated herein is provided.

In particular embodiments, the anti-CD20 CCR comprises an anti-CD20 antibody or antigen binding fragment thereof, a second transmembrane domain, and a second intracellular costimulatory domain.

In additional embodiments, the anti-CD20 CCR comprises an anti-CD20 antibody or antigen binding fragment thereof selected from the group consisting of: a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, Nanobody).

In various embodiments, the anti-CD20 CCR comprises an anti-CD20 antibody or antigen binding fragment thereof that is an scFv.

In other embodiments, the anti-CD20 CCR comprises an anti-CD20 antibody or antigen binding fragment thereof comprising a variable light chain sequence comprising CDRL1-CDRL3 sequences set forth in SEQ ID NOs: 25-27 and a variable heavy chain sequence comprising CDRH1-CDRH3 sequences set forth in SEQ ID NOs: 28-30.

In certain embodiments, the anti-CD20 CCR comprises an anti-CD20 antibody or antigen binding fragment thereof comprising a variable light chain sequence as set forth in SEQ ID NO: 31 and a variable heavy chain sequence as set forth in SEQ ID NO: 32.

In particular embodiments, the anti-CD20 CCR comprises a second transmembrane domain isolated from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1.

In some embodiments, the anti-CD20 CCR comprises a second transmembrane domain isolated from CD8α.

In various embodiments, the anti-CD20 CCR comprises a second costimulatory signaling domain isolated from a costimulatory molecule selected from the group consisting of: TLR1, TLR2, TLR3, TLR4, TLR5, TLR6, TLR7, TLR8, TLR9, TLR10, CARD 11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

In various embodiments, the anti-CD20 CCR comprises a second costimulatory signaling domain is isolated from CD28.

In certain embodiments, the anti-CD20 CCR comprises a CD8a hinge, a CD8α transmembrane domain, and a CD28 costimulatory signaling domain.

In particular embodiments, a fusion polypeptide comprising an anti-CD79A CAR comprising the amino acid sequence set forth in any one of SEQ ID NOs: 17-20, a T2A self-cleaving polypeptide, and an anti-CD20 CCR comprising the amino acid sequence set forth in SEQ ID NO: 33 or SEQ ID NO: 35 is provided.

In further embodiments, a fusion polypeptide comprises the amino acid sequence set forth in SEQ ID NO: 37 or SEQ ID NO: 39.

In particular embodiments, a polynucleotide encoding an anti-CD79A CAR and an anti-CD20 CAR contemplated herein is provided.

In some embodiments, a polynucleotide encoding a fusion polypeptide contemplated herein is provided.

In various embodiments, a polynucleotide is provided comprising a sequence set forth in SEQ ID NO: 38 or SEQ ID NO: 40.

In some embodiments, a vector comprising a polynucleotide contemplated herein is provided.

In particular embodiments, the vector is an expression vector.

In other embodiments, the vector is an episomal vector.

In particular embodiments, the vector is a viral vector.

In certain embodiments, the vector is a retroviral vector.

In various embodiments, the vector is a lentiviral vector.

In various embodiments, a cell that expresses a fusion polypeptide contemplated herein is provided.

In particular embodiments, a cell comprising a polynucleotide encoding a fusion polypeptide contemplate herein or a cell comprising a vector contemplated herein is provided.

In various embodiments, a cell is provided comprising one or more polynucleotides encoding: an anti-CD79A CAR comprising an anti-CD79A antibody or antigen binding fragment thereof, a first transmembrane domain; a first intracellular costimulatory signaling domain; and a primary signaling domain; and an anti-CD20 CCR comprising an anti-CD20 antibody or antigen binding fragment thereof, a second transmembrane domain; a second intracellular costimulatory signaling domain.

In particular embodiments, the anti-CD79A antibody or antigen binding fragment thereof and the anti-CD20 antibody or antigen binding fragment thereof are both independently selected from the group consisting of: a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, Nanobody).

In certain embodiments, the anti-CD79A antibody or antigen binding fragment thereof and the anti-CD20 antibody or antigen binding fragment thereof are both scFvs.

In various embodiments, the anti-CD79A antibody or antigen binding fragment thereof comprises a variable light chain sequence comprising CDRL1-CDRL3 sequences set forth in SEQ ID NOs: 1-3 or 9-11 and a variable heavy chain sequence comprising CDRH1-CDRH3 sequences set forth in SEQ ID NOs: 4-6 or 12-14.

In other embodiments, the anti-CD79A antibody or antigen binding fragment thereof comprises the light chain CDRs as set forth in SEQ ID NOs: 1-3 and the heavy chain CDRs as set forth in SEQ ID NOs: 4-6.

In particular embodiments, the anti-CD79A antibody or antigen binding fragment thereof comprises the light chain CDRs as set forth in SEQ ID NOs: 9-11 and the heavy chain CDRs as set forth in SEQ ID NOs: 12-14.

In various embodiments, the anti-CD79A antibody or antigen binding fragment thereof comprises a variable light chain sequence as set forth in any one of SEQ ID NOs: 7 or 15 and a variable heavy chain sequence as set forth in any one of SEQ ID NOs: 8 or 16.

In some embodiments, the anti-CD79A antibody or antigen binding fragment thereof comprises a variable light chain sequence as set forth in SEQ ID NO: 7 and/or a variable heavy chain sequence as set forth in SEQ ID NO: 8.

In additional embodiments, the anti-CD79A antibody or antigen binding fragment thereof comprises a variable light chain sequence as set forth in SEQ ID NO: 15 and/or a variable heavy chain sequence as set forth in SEQ ID NO: 16.

In certain embodiments, the anti-CD20 antibody or antigen binding fragment thereof comprises a variable light chain sequence comprising CDRL1-CDRL3 sequences set forth in SEQ ID NOs: 25-27 and a variable heavy chain sequence comprising CDRH1-CDRH3 sequences set forth in SEQ ID NOs: 28-30.

In various embodiments, the anti-CD20 antibody or antigen binding fragment thereof comprising a variable light chain sequence as set forth in SEQ ID NO: 31 and a variable heavy chain sequence as set forth in SEQ ID NO: 32.

In particular embodiments, the first transmembrane domain and the second transmembrane domain are each independently isolated from a polypeptide selected from the group consisting of alpha or beta chain of the T-cell receptor, CDδ, CD3ε, CDγ, CD3ζ, CD4, CD5, CD8a, CD9, CD 16, CD22, CD27, CD28, CD33, CD37, CD45, CD64, CD80, CD86, CD 134, CD137, CD152, CD154, and PD1.

In further embodiments, the first transmembrane domain and the second transmembrane domain are both isolated from CD8a.

In various embodiments, the first costimulatory signaling domain and the second costimulatory domain are each independently isolated from a costimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

In certain embodiments, the first costimulatory signaling domain isolated from CD137.

In other embodiments, the primary signaling domain isolated from a polypeptide selected from the group consisting of: FcRγ, FcRβ, CD3γ, CD3δ, CD3ε, CD3ζ, CD22, CD79a, CD79b, and CD66d.

In particular embodiments, the primary signaling domain isolated from CD3ζ.

In various embodiments, the second costimulatory signaling domain isolated from CD28.

In particular embodiments, the anti-CD20 CCR comprises a CD8a hinge domain, a CD8α transmembrane domain, and a CD28 costimulatory signaling domain.

In particular embodiments, the cell expresses an anti-CD79A CAR comprising an amino acid sequence set forth in any one of SEQ ID NOs: 17-20 and an anti-CD20 CCR comprising an amino acid sequence set forth in SEQ ID NO: 33 or SEQ ID NO: 35.

In some embodiments, the cell comprises a first polynucleotide encoding the anti-CD79A CAR and a second polynucleotide encoding the anti-CD20 CCR.

In various embodiments, an isolated polynucleotide encodes the anti-CD79A CAR and the anti-CD20 CCR.

In some embodiments, the isolated polynucleotide encodes the anti-CD79A CAR, an IRES sequence, and the anti-CD20 CCR.

In particular embodiments, the isolated polynucleotide encodes the anti-CD79A CAR, a polypeptide cleavage signal, and the anti-CD20 CCR.

In various embodiments, the polypeptide cleavage signal is a viral self-cleaving polypeptide.

In certain embodiments, the polypeptide cleavage signal is a viral self-cleaving 2A polypeptide.

In various embodiments, the polypeptide cleavage signal is a viral self-cleaving polypeptide selected from the group consisting of: a foot-and-mouth disease virus (FMDV) 2A (F2A) peptide, an equine rhinitis A virus (ERAV) 2A (E2A) peptide, a Thosea asigna virus (TaV) 2A (T2A) peptide, a porcine teschovirus-1 (PTV-1) 2A (P2A) peptide, a Theilovirus 2A peptide, and an encephalomyocarditis virus 2A peptide.

In particular embodiments, the cell comprises insertion or deletion of one or more nucleotides in a homing endonuclease (HE) variant cleavage target site or a megaTAL cleavage target site in the casitas B-lineage (Cbl) lymphoma proto-oncogene B (CBLB) gene.

In certain embodiments, the HE variant introduces one or more insertions or deletions into the HE target site in the CBLB gene set forth in SEQ ID NO: 55.

In some embodiments, the megaTAL introduces one or more insertions or deletions into the megaTAL target site in the CBLB gene set forth in SEQ ID NO: 56.

In particular embodiments, the insertions or deletions in the CBLB gene decrease CBLB expression, function, and/or activity.

In further embodiments, the cell comprises one or more modified CBLB alleles.

In additional embodiments, the cell comprises one or more modified CBLB alleles that do not express or produce CBLB or that express or produce non-functional CBLB.

In particular embodiments, the cell comprises insertion or deletion of one or more nucleotides in a homing endonuclease (HE) variant cleavage target site or a megaTAL cleavage target site in the programmed cell death 1 (PDCD-1) gene or.

In some embodiments, the HE variant introduces one or more insertions or deletions into the HE target site in the PDCD-1 gene set forth in SEQ ID NO: 51.

In further embodiments, the megaTAL introduces one or more insertions or deletions into the megaTAL target site in the PDCD-1 gene set forth in SEQ ID NO: 52.

In particular embodiments, the insertions or deletions in the PDCD-1 gene decrease PDCD-1 expression, function, and/or activity.

In some embodiments, the cell comprises one or more modified PDCD-1 alleles.

In particular embodiments, the cell comprises one or more modified PDCD-1 alleles that do not express or produce PDCD-1 or that express or produce non-functional PDCD-1.

In further embodiments, the cell is a hematopoietic cell.

In certain embodiments, the cell is a hematopoietic stem or progenitor cell.

In other embodiments, the cell is a CD34⁺ hematopoietic stem or progenitor cell.

In particular embodiments, the cell is an immune effector cell.

In various embodiments, the cell is a T cell.

In certain embodiments, the cell is a CD3⁺, CD4⁺, and/or CD8⁺ cell.

In particular embodiments, the cell is a cytotoxic T lymphocytes (CTLs), a tumor infiltrating lymphocytes (TILs), or a helper T cell.

In some embodiments, the cell is a natural killer (NK) cell or natural killer T (NKT) cell.

In various embodiments, a population of cells is provided comprising a plurality of cells contemplated herein.

In further embodiments, a population of cells comprising one or more hematopoietic stem or progenitor cells and one or more immune effector cells contemplated herein is provided.

In various embodiments, a population of cells comprising one or more CD34⁺ hematopoietic stem or progenitor cells and one or more T cells contemplated herein is provided.

In certain embodiments, a composition comprises the genetically modified cells contemplated herein and a physiologically acceptable excipient.

In some embodiments, a composition comprises a population of cells contemplated herein and a physiologically acceptable excipient.

In particular embodiments, method for killing cancer cells that express CD79A or CD20 in a subject is provided, comprising administering to the subject therapeutically effective amount of a composition contemplated herein.

In particular embodiments, method for killing cancer cells that express CD79A and CD20 in a subject is provided, comprising administering to the subject therapeutically effective amount of a composition contemplated herein.

In particular embodiments, method for killing cancer cells that express CD79A and/or CD20 in a subject is provided, comprising administering to the subject therapeutically effective amount of a composition contemplated herein.

In various embodiments, a method for decreasing the number of cancer cells that express CD79A and CD20 in a subject is provided, comprising administering to the subject therapeutically effective amount of a composition contemplated herein sufficient to decrease the number of cancer cells that express CD79A and CD20 compared to the number of the cancer cells that express CD79A and CD20 prior to the administration.

In various embodiments, a method for decreasing the number of cancer cells that express CD79A or CD20 in a subject is provided, comprising administering to the subject therapeutically effective amount of a composition contemplated herein sufficient to decrease the number of cancer cells that express CD79A or CD20 compared to the number of the cancer cells that express CD79A or CD20 prior to the administration.

In various embodiments, a method for decreasing the number of cancer cells that express CD79A and/or CD20 in a subject is provided, comprising administering to the subject therapeutically effective amount of a composition contemplated herein sufficient to decrease the number of cancer cells that express CD79A and/or CD20 compared to the number of the cancer cells that express CD79A and/or CD20 prior to the administration.

In some embodiments, a method of treating a cancer in a subject in need thereof is provided, comprising administering to the subject a therapeutically effect amount of a composition contemplated herein.

In particular embodiments, the cancer is a solid cancer.

In other embodiments, the solid cancer is an osteosarcoma or Ewing's sarcoma.

In certain embodiments, the cancer is a liquid cancer.

In some embodiments, the cancer is a hematological malignancy.

In various embodiments, the cancer is non-Hodgkin's lymphoma, acute lymphocytic leukemia (ALL), chronic lymphocytic leukemia (CLL), hairy cell leukemia (HCL), multiple myeloma (MM), acute myeloid leukemia (AML), or chronic myeloid leukemia (CML).

In particular embodiments, the non-Hodgkin's lymphoma is Burkitt's lymphoma, small lymphocytic lymphoma (SLL), diffuse large B cell lymphoma (DLBCL), follicular lymphoma (FL), mantle cell lymphoma (MCL), or marginal zone lymphoma (MZL).

In various embodiments, the non-Hodgkin's lymphoma is diffuse large B cell lymphoma (DLBCL).

In some embodiments, the cancer is a MM selected from the group consisting of: overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In various embodiments, a method for treating a subject that has DLBCL is provided comprising administering to the subject a therapeutically effect amount of a composition contemplated herein.

In particular embodiments, a method for ameliorating at one or more symptoms associated with a cancer expressing CD79A and/or CD20 in a subject is provided, comprising administering to the subject a therapeutically effective amount of a composition contemplated herein sufficient to ameliorate at least one symptom associated with cancer cells that express CD79A and/or CD20.

In certain embodiments, the one or more symptoms ameliorated are selected from the group consisting of: weakness, fatigue, shortness of breath, easy bruising and bleeding, frequent infections, enlarged lymph nodes, distended or painful abdomen, bone or joint pain, fractures, unplanned weight loss, poor appetite, night sweats, persistent mild fever, and decreased urination.

In particular embodiments, a method of generating a population of cells that expresses a fusion polypeptide contemplated herein is provided comprising introducing into a population of cells, a polynucleotide, or a vector contemplated herein.

In various embodiments, a method of generating a population of cells that expresses an anti-CD79A CAR and an anti-CD20 CCR is provided comprising introducing into the population of cells one or more polynucleotides encoding an anti-CD79A CAR and an anti-CD20 CCR contemplated herein.

In some embodiments, a method of generating a population of cells that expresses an anti-CD79A CAR and an anti-CD20 CCR is provided comprising introducing into a population of cells a polynucleotide encoding a fusion polypeptide contemplated herein.

In some embodiments, a method of generating a population of cells that expresses an anti-CD79A CAR and an anti-CD20 CCR is provided comprising introducing into a population of cells a polynucleotide encoding a fusion polypeptide sequence set forth in SEQ ID NO: 37 or SEQ ID NO: 39.

In certain embodiments, a method of generating a population of cells that expresses an anti-CD79A CAR and an anti-CD20 CCR is provided comprising introducing into the population of cells a first polynucleotide encoding the anti-CD79A CAR set forth in any one of SEQ ID NOs: 17-20 and a second polynucleotide encoding the ani-CD20 CCR sequence set forth in SEQ ID NO: 33 or SEQ ID NO: 35.

In particular embodiments, a method of generating a population of cells that expresses an anti-CD79A CAR and an anti-CD20 CCR is provided comprising introducing into the population of cells the polynucleotide sequence set forth in SEQ ID NO: 38 or SEQ ID NO: 40.

In various embodiments, a method of generating a population of cells that expresses an anti-CD79A CAR and an anti-CD20 CCR is provided comprising introducing into the population of cells a first polynucleotide sequence set forth in any one of SEQ ID NOs: 21 to 24 and a second polynucleotide sequence set forth in SEQ ID NO: 34 or SEQ ID NO: 36.

In particular embodiments, one or more cells in the population of cells comprises one or more insertions or deletions in the PDCD-1 gene at a polynucleotide sequence set forth in SEQ ID NO: 51 that decrease or eliminate PDCD-1 expression and/or function.

In certain embodiments, a polynucleotide encoding an HE variant that binds and cleaves the polynucleotide sequence set forth in SEQ ID NO: 51 is introduced into the population of cells.

In particular embodiments, one or more cells in the population of cells comprises one or more insertions or deletions in the PDCD-1 gene at a polynucleotide sequence set forth in SEQ ID NO: 52 that decrease or eliminate PDCD-1 expression and/or function.

In some embodiments, a polynucleotide encoding a megaTAL that binds and cleaves the polynucleotide sequence set forth in SEQ ID NO: 52 is introduced into the population of cells.

In certain embodiments, one or more cells in the population of cells comprises one or more insertions or deletions in the CBLB gene at a polynucleotide sequence set forth in SEQ ID NO: 55 that decrease or eliminate CBLB expression and/or function.

In particular embodiments, a polynucleotide encoding an HE variant that binds and cleaves the polynucleotide sequence set forth in SEQ ID NO: 55 is introduced into the population of cells.

In some embodiments, one or more cells in the population of cells comprises one or more insertions or deletions in the CBLB gene at a polynucleotide sequence set forth in SEQ ID NO: 56 that decrease or eliminate CBLB expression and/or function.

In particular embodiments, a polynucleotide encoding a megaTAL that binds and cleaves the polynucleotide sequence set forth in SEQ ID NO: 56 is introduced into the population of cells.

In some embodiments, the population of cells comprises hematopoietic stem or progenitor cells.

In certain embodiments, the population of cells comprises CD34⁺ hematopoietic stem or progenitor cells.

In various embodiments, the population of cells comprises immune effector cells.

In particular embodiments, the population of cells comprises T cells, NK cells, and/or NKT cells.

In various embodiments, the population of cells comprises T cells.

### BRIEF DESCRIPTION OF SEVERAL VIEWS OF THE DRAWINGS

FIGS. 1A-1C shows anti-CD79A CAR and anti-CD20 CCR expression and activity. A) CD79A CAR expression on untransduced PBMCs (UTD) or PBMCs transduced with lentiviral vector encoding an anti-CD79A CAR, T2A, anti-CD20 CCR fusion protein. B) This panel shows IFNγ secretion of UTD T cells or T cells expressing anti-CD79A CAR and anti-CD20 CCR co-cultured in the absence of target cells or with RD cells (CD79A⁻, CD20⁻), RD.CD79A cells (CD79A⁺, CD20), RD.CD79A.CD20 cells (CD79A⁺, CD20⁺), or Daudi cells (CD79A⁺, CD20⁺, high expression). C) This panel shows IL-2 secretion of UTD T cells, antiCD79A CAR T cells, or T cells expressing anti-CD79A CAR and anti-CD20 CCR co-cultured in the absence of target cells or with RD cells (CD79A⁻, CD20), RD.CD79A cells (CD79A⁺, CD20⁻), RD.CD79A.CD20 cells (CD79A⁺, CD20⁺), or Daudi cells (CD79A⁺, CD20⁺, high expression).

FIGS. 2A and 2B shows anti-CD79A CAR and anti-CD20 CCR activity against RD.CD20 cells. A) This panel shows IFNγ secretion of UTD T cells, anti-CD79 CAR T cells, or T cells expressing anti-CD79A CAR and anti-CD20 CCR co-cultured with RD cells (CD79A⁻, CD20⁻), or RD.CD20 cells (CD79A⁻, CD20⁺). C) This panel shows IL-2 secretion of UTD T cells, anti-CD79 CAR T cells, or T cells expressing anti-CD79A CAR and anti-CD20 CCR co-cultured with RD cells (CD79A⁻, CD20⁻), or RD.CD20 cells (CD79A⁻, CD20⁺).

FIGS. 3A and 3B shows anti-CD79A CAR and anti-CD20 CAR expression and activity. A) CD79A CAR expression on untransduced PBMCs (UTD) or PBMCs transduced with a lentiviral vector encoding an anti-CD79A-CD8α-4-1BB-CD3ζ, T2A, anti-CD20-CD8α-CD28-CD3ζ fusion protein (middle panel), or a lentiviral vector encoding an anti-CD79A-CD8α-4-1BB-CD3ζ, T2A, anti-CD20-CD8α-4-1BB-CD3 fusion protein (right panel). B) This panel shows IFNγ secretion of UTD T cells or T cells expressing an anti-CD79A-CD8α-4-1BB-CD3ζ, T2A, anti-CD20-CD8α-CD28-CD3ζ fusion protein, or an anti-CD79A-CD8α-4-1BB-CD3ζ, T2A, anti-CD20-CD8α-4-1BB-CD3 fusion protein co-cultured in the absence of target cells or with RD cells (CD79A⁻, CD20⁻), RD.CD79A cells (CD79A⁺, CD20⁻), RD. CD20 cells (CD79A⁻, CD20⁺), or Daudi cells (CD79A⁺, CD20⁺, high expression).

FIGS. 4A-4C shows anti-CD79A CAR and anti-CD20 CCR expression and activity. A) CD79A CAR expression on untransduced PBMCs (UTD) or PBMCs transduced with lentiviral vector encoding an anti-CD79A CAR, T2A, anti-CD20 CCR fusion protein. B) This panel shows IFNγ secretion of UTD T cells or T cells expressing anti-CD79A CAR and anti-CD20 CCR co-cultured in the absence of target cells or with RD cells (CD79A⁻, CD20-), RD.CD79A cells (CD79A⁺, CD20⁻), or REC-1 cells (CD79A⁺, CD20⁺, high expression). B) This panel shows IL-2 secretion of UTD T cells, antiCD79A CAR T cells, or T cells expressing anti-CD79A CAR and anti-CD20 CCR co-cultured in the absence of target cells or with RD cells (CD79A⁻, CD20⁻), RD.CD79A cells (CD79A⁺, CD20⁻), RD.CD79A.CD20 cells (CD79A⁺, CD20⁺), or REC-1 cells (CD79A⁺, CD20⁺, high expression).

FIGS. 5A and 5B shows anti-CD79A CAR and anti-CD20 CCR activity against RD.CD20 cells. A) This panel shows IFNγ secretion of UTD T cells, anti-CD79 CAR T cells, or T cells expressing anti-CD79A CAR and anti-CD20 CCR co-cultured with RD cells (CD79A⁻, CD20⁻), or RD.CD20 cells (CD79A⁻, CD20⁺). C) This panel shows IL-2 secretion of UTD T cells, anti-CD79 CAR T cells, or T cells expressing anti-CD79A CAR and anti-CD20 CCR co-cultured with RD cells (CD79A⁻, CD20⁻), or RD.CD20 cells (CD79A⁻, CD20⁺).

FIGS. 6A and 6B shows anti-CD79A CAR and anti-CD20 CAR expression and activity. A) CD79A CAR expression on untransduced PBMCs (UTD) or PBMCs transduced with a lentiviral vector encoding an anti-CD79A-CD8α-4-1BB-CD3ζ, T2A, anti-CD20-CD8α-CD28-CD3ζ fusion protein (middle panel), or a lentiviral vector encoding an anti-CD79A-CD8α-4-1BB-CD3ζ, T2A, anti-CD20-CD8α-4-1BB-CD3 fusion protein (right panel). B) This panel shows IFNγ secretion of UTD T cells or T cells expressing an anti-CD79A-CD8α-4-1BB-CD3ζ, T2A, anti-CD20-CD8α-CD28-CD3ζ fusion protein, or an anti-CD79A-CD8α-4-1BB-CD3ζ, T2A, anti-CD20-CD8α-4-1BB-CD3 fusion protein co-cultured in the absence of target cells or with RD cells (CD79A⁻, CD20⁻), RD.CD79A cells (CD79A⁺, CD20⁻), RD. CD20 cells (CD79A⁻, CD20⁺), or Daudi cells (CD79A⁺, CD20⁺, high expression).

FIG. 7 shows cytotoxicity of T cells that express an anti-CD79A CAR and an anti-CD20 CCR against cell lines engineered to express CD79A or CD20. Untransduced PBMCs (UTD) or PBMCs transduced with lentiviral vector encoding an anti-CD79A CAR, T2A, anti-CD20 CCR fusion protein were co-cultured in the presence of RD cells (CD79A⁻, CD20-; left panel), RD.79A cells (CD79A⁺, CD20; center panel), and RD.20 cells (CD79A⁻, CD20⁺; right panel).

FIG. 8 shows efficacy of T cells expressing an anti-CD79A CAR and an anti-CD20 CCR against Daudi cells (CD79A⁺, CD20⁺, high expression) in vitro and in vivo. Untransduced PBMCs (UTD) or PBMCs transduced with lentiviral vector encoding an anti-CD79A CAR, T2A, anti-CD20 CCR fusion protein were co-cultured in the presence of Daudi cells at a 1:1 ratio for 24 hours and supernatants were collected and analyzed for IFNγ using Luminex (left panel, n=3). NSG mice were intravenously injected with 2×10⁶ luciferase-expressing Daudi cells; after 13 days five mice were injected with Vehicle (medium), 10×10⁶ UTD T cells or 10×10⁶ T cells expressing the anti-CD79A CAR and anti-CD20 CCR; and mice were monitored for 30 days (right panel).

FIG. 9 shows efficacy of T cells expressing an anti-CD79A CAR and an anti-CD20 CCR against NU-DUL-1 cells (DLBCL tumor model) in vitro and in vivo. Untransduced PBMCs (UTD) or PBMCs transduced with lentiviral vector encoding an anti-CD79A CAR, T2A, anti-CD20 CCR fusion protein were co-cultured in the presence of NU-DUL-1 cells at a 1:1 ratio for 24 hours and supernatants were collected and analyzed for IFNγ using Luminex (left panel, n=3). NSG mice were intravenously injected with 2×10⁶ luciferase-expressing NU-DUL-1 cells; after 15 days five mice were injected with Vehicle (medium), 10×10⁶ UTD T cells or 5×10⁶ T cells expressing the anti-CD79A CAR and anti-CD20 CCR; and mice were monitored for 30 days (right panel).

FIG. 10 shows efficacy of T cells expressing an anti-CD79A CAR and an anti-CD20 CCR against a Toledo (Germinal Center B Cell (GCB) DLBCL tumor model in vitro and in vivo. Untransduced PBMCs (UTD) or PBMCs transduced with lentiviral vector encoding an anti-CD79A CAR, T2A, anti-CD20 CCR fusion protein were co-cultured in the presence of GCB DLBCL cells at a 1:1 ratio for 24 hours and supernatants were collected and analyzed for IFNγ using Luminex (left panel, n=3). NSG mice were intravenously injected with 50×10⁶ luciferase-expressing GCB DLBCL cells; after 16 days (tumors at 100 mm³) five mice were injected with Vehicle (medium), 20×10⁶ UTD T cells or 2.5×10⁶ T cells expressing the anti-CD79A CAR and anti-CD20 CCR; and mice were monitored for 30 days (right panel).

FIG. 11 shows efficacy of T cells expressing an anti-CD79A CAR and an anti-CD20 CCR in the presence a CBLB edit against a Toledo (Germinal Center B Cell (GCB) DLBCL tumor model in vitro and in vivo. Untransduced PBMCs (UTD) or PBMCs transduced with lentiviral vector encoding an anti-CD79A CAR, T2A, anti-CD20 CCR fusion protein with and without genome editing at the CBLB locus were co-cultured in the presence of GCB DLBCL cells at a 1:1 ratio for 24 hours and supernatants were collected and analyzed for IFNγ using Luminex (left panel, n=3). NSG mice were intravenously injected with 50×10⁶ luciferase-expressing GCB DLBCL cells; after 17 days (tumors at 130 mm³) five mice were injected with Vehicle (medium), 5×10⁶ UTD T cells+/−CBLB edit or 1×10⁶ T cells expressing the anti-CD79A CAR and anti-CD20 CCR+/−CBLB edit; and mice were monitored for 21 days (right panel).

FIG. 12 shows efficacy of T cells expressing an anti-CD79A CAR and an anti-CD20 CCR in the presence a CBLB edit against a Daudi.CD20KO (CD20 knockout) tumor model in vivo. NSG mice were intravenously injected with 2×10⁶ luciferase-expressing Daudi.CD20KO cells; after 14 days five mice were injected with Vehicle (medium), 20×10⁶ UTD T cells+/−CBLB edit or 10×10⁶ T cells expressing the anti-CD79A CAR and anti-CD20 CCR+/−CBLB edit; and mice were monitored for 30 days.

FIG. 13 shows cytokine secretion in T cells expressing an anti-CD79A CAR and an anti-CD20 CCR in the presence a CBLB edit and activated using anti-CD3 and anti-CD28 antibodies. 1×10⁶ cells/mL of UTD T cells+/−CBLB edit or T cells expressing an anti-CD79A CAR and anti-CD20 CCR were cultured in 96-well high binding plates coated with monoclonal antibodies against CD3 (titrated from 1 μg/mL to 0.063 μg/mL) and CD28 (5 μg/mL) for 24 hours, 11-2 (left panel) and IFNγ (right panel) were measured via Luminex (n=3).

FIG. 14 shows enhanced IL-2 secretion in T cells expressing an anti-CD79A CAR and an anti-CD20 CCR in the presence a CBLB edit. UTD T cells+/−CBLB edit and T cells expressing the anti-CD79A CAR and anti-CD20 CCR+/−CBLB were co-cultured at a 1:1 ratio with Daudi (Burkitt Lymphoma; CD79⁺, CD20⁺) tumor cells for 24 hours and supernatants were collected and analyzed for IL-2 using Luminex.

FIG. 15 shows that T cells treated with a CBLB megaTAL exhibit enhanced proliferation in a serial restimulation assay compared to T cells treated with the TCRα dead megaTAL. UTD T cells or T cells transduced with a single lentiviral vector encoding an anti-CD79A CAR and an anti-CD20 CCR were treated with a CBLB megaTAL or a TCRα dead megaTAL and subjected to a serial restimulation assay (n=4).

FIG. 16 shows an enhanced cytokine response of T cells expressing an anti-CD79A CAR and an anti-CD20 CCR in the presence a CBLB edit against a GCB DLBCL tumor model. PBMCs from 3 healthy donors (DH) and 3 DLBCL donors (DL) transduced with a lentiviral vector encoding an anti-CD79A CAR and an anti-CD20 CCR were co-cultured at a 1:1 ratio with Toledo GCB DLBCL tumor cells for 24 hours and supematants were collected and analyzed for IFNγ using Luminex.

### BRIEF DESCRIPTION OF THE SEQUENCE IDENTIFIERS

SEQ ID NOs: 1-16 set forth amino acid sequences of exemplary light chain CDR sequences, heavy chain CDR sequences, variable domain light chains, and variable domain heavy chains for anti-CD79A CARs contemplated herein.

SEQ ID NOs: 17-20 set forth the amino acid sequences of exemplary anti-CD79A CARs.

SEQ ID NOs: 21-24 set forth the nucleic acid sequences of exemplary anti-CD79A CARs.

SEQ ID NOs: 25-32 set forth amino acid sequences of exemplary light chain CDR sequences, heavy chain CDR sequences, variable domain light chains, and variable domain heavy chains for anti-CD20 CCRs contemplated herein.

SEQ ID NOs: 33 and 35 set forth the amino acid sequences of exemplary anti-CD20 CCRs.

SEQ ID NOs: 34 and 36 set forth the nucleic acid sequences of exemplary anti-CD20 CCRs.

SEQ ID NOs: 37 and 39 set forth the amino acid sequences of exemplary anti-CD79A CAR-T2A-anti-CD20 CCR fusion proteins.

SEQ ID NOs: 38 and 40 set forth the nucleic acid sequences of exemplary anti-CD79A CAR-T2A-anti-CD20 CCR fusion proteins.

SEQ ID NOs: 41 and 43 set forth the amino acid sequences of exemplary anti-CD79A CAR-T2A-anti-CD20 CD28z CAR fusion proteins.

SEQ ID NOs: 42 and 44 set forth the nucleic acid sequences of exemplary anti-CD79A CAR-T2A-anti-CD20 CD28z CAR fusion proteins.

SEQ ID NOs: 45 and 47 set forth the amino acid sequences of exemplary anti-CD79A CAR-T2A-anti-CD20 BBz CAR fusion proteins.

SEQ ID NOs: 46 and 48 set forth the nucleic acid sequences of exemplary anti-CD79A CAR-T2A-anti-CD20 BBz CAR fusion proteins.

SEQ ID NO: 49 is an amino acid sequence of an I-OnuI LHE variant reprogrammed to bind and cleave a target site in the human PDCD-1 gene.

SEQ ID NO: 50 is an amino acid sequence of a megaTAL that binds and cleaves a target site in a human PDCD-1 gene.

SEQ ID NO: 51 is an I-OnuI LHE variant target site in a human PDCD-1 gene.

SEQ ID NO: 52 is a megaTAL target site in a human PDCD-1 gene.

SEQ ID NO: 53 is an amino acid sequence of an I-OnuI LHE variant reprogrammed to bind and cleave a target site in the human CBLB gene.

SEQ ID NO: 54 is an amino acid sequence of a megaTAL that binds and cleaves a target site in a human CBLB gene.

SEQ ID NO: 55 is an I-OnuI LHE variant target site in a human CBLB gene.

SEQ ID NO: 56 is a megaTAL target site in a human CBLB gene.

SEQ ID NOs: 57-67 set forth the amino acid sequences of various linkers.

SEQ ID NOs: 68-92 set forth the amino acid sequences of protease cleavage sites and self-cleaving polypeptide cleavage sites. In the foregoing sequences, X, if present, refers to any amino acid or the absence of an amino acid.

### DETAILED DESCRIPTION

### A. Overview

Cancers are often heterogeneous pools of cells expressing different levels of various antigens. Generally, immunotherapies are initially selected to target an antigen that is expressed on a majority of cancer cells and that substantially lacks expression on normal cells. An effective targeted immunotherapy will kill the majority of cancer cells that express the target antigen, resulting in partial or complete remission. However, because most cancers are heterogeneous in nature, the remaining cancer cells that do not express, or that express low levels, of the targeted antigen are spared and can potentially give rise to cancer cells that are not effectively targeted by the initial immunotherapy.

One major obstacle that still limits the efficacy of adoptive cell therapy is relapse of “antigen negative” cancers. The alarmingly high rate of antigen negative relapse represents an, as of yet, unmet need of adoptive cell therapy. Without wishing to be bound by any particular theory, the inventors have solved the problem killing cancers heterogenous for expression of multiple target antigens by re-engineering immune effector cells (e.g., T cells, NK cells) to express chimeric antigen receptors (CARs) and chimeric costimulatory receptors (CCRs) that target multiple antigens and synergistically activate multiple intracellular cell signaling pathways to boost the inflammatory cytokine response and ability to kill and prevent relapse of tumor cells bearing one or both of the target antigens. Surprisingly, the inventors have discovered that immune effector cells that express a CAR and a CCR directed against different antigens do not require the presence of both antigens on the target cell in order to elicit an inflammatory cytokine response and kill the cells. This is surprising because the CCR does not contain a signaling domain, and thus, theoretically should not be able to signal on its own. Thus, the compositions and methods contemplated herein represent an important advance in T cell immunotherapy against heterogenous cancers.

Another obstacle that limits the efficacy of adoptive cell therapy is the hyporesponsiveness of immune effector cells due to exhaustion mediated by the tumor microenvironment. For example, exhausted T cells have a unique molecular signature that is markedly distinct from naive, effector or memory T cells. They are defined as immune effector cells with decreased cytokine expression and effector function. Programmed cell death 1 (PDCD-1) is a T cell exhaustion marker; increased PD-1 expression is associated with decreased T cell proliferation and reduced production of IL-2, TNF, and IFN-γ. Casitas B-lineage (Cbl) lymphoma proto-oncogene B (CBLB) is a member of the RING-finger family or E3 ubiquitin ligases that is involved in the negative regulation of effector T cell activity and persistence. CBLB knockout mouse T cells are hyperproliferative, produce heightened levels of IL2 and IFNγ in response to antigen stimulation, are resistant to TGFβ-mediated suppression, and have a lower activation threshold indicating that CBLB plays a role in negatively regulating T cell activation. Without wishing to be bound by any particular theory, it is contemplated that disruption of the PDCD-1 and/or CBLB genes in immune effector cells that express a CAR directed against a first antigen and a CCR directed against a second antigen results in a more efficacious and persistent adoptive cell therapy.

The invention generally relates to improved compositions and methods for preventing or treating cancers that express CD79A and/or CD20 or preventing, treating, or ameliorating at least one symptom associated with an CD79A and/or CD20 expressing cancer. In various embodiments, the invention relates to improved adoptive cell therapy of cancers that express CD79A and/or CD20 using genetically modified immune effector cells, wherein the immune effector cells optionally comprise one or more genome edits that decrease or eliminate the expression and/or function of PDCD1 and/or CBLB. Genetic approaches offer a potential means to enhance immune recognition and elimination of cancer cells. Immune effector cells modified to express a chimeric antigen receptors (CAR) and a chimeric costimulatory receptor (CCR) are contemplated, in particular embodiments, to redirect cytotoxicity toward cancer cells expressing either the CAR target antigen or the CCR target antigen and synergistically enhance the immune effector cell response to the cancers. In particular preferred embodiments, immune effector cells modified to express a CAR and CCR further comprise one or more genome edits that reduce or eliminate the expression and/or function of PDCD-1 and/or CBLB. In other particular preferred embodiments, immune effector cells modified to express a CAR and CCR further comprise one or more genome edits that reduce or eliminate the expression and function of CBLB.

The improved compositions and methods of adoptive cell therapy contemplated in particular embodiments herein, provide genetically modified immune effector cells that can readily be expanded, exhibit long-term persistence in vivo, and demonstrate antigen dependent cytotoxicity to cells expressing CD79A and/or CD20, and resistance to the immunosuppressive signals in the tumor microenvironment.

CD79A is also known as B cell antigen receptor complex-associated protein alpha chain, Membrane-Bound Immunoglobulin-Associated Protein (MB1, MB-1), surface IgM-associated protein, and Ig-alpha (IGA). Illustrative examples of polynucleotide sequences encoding CD79A include, but are not limited to: NM_001783.3, NM_021601.3, ENST00000221972 (uc002orv.3), ENST00000597454 (uc060zdj.1), ENST00000444740 (uc002oru.4), Hs.631567, and AK223371. Illustrative examples of polypeptide sequences encoding CD79a include, but are not limited to: P11912-1, P11912-2, ENSP00000400605 ENSP00000468922, ENSP00000221972, NP_001774.1, and NP_067612.1.

CD79 consist of two proteins, namely CD79A and CD79B. CD79A is located at chromosome 19q13.2 and encodes a 226-amino-acid glycoprotein of approximately 47 kDa. The exact molecular weight depends on the extent of glycosylation. CD79B is located at chromosome 17q23 and encodes a 229-amino-acid glycoprotein of approximately 37 kDa. CD79A and CD79B share an exon-intron structure, both contain a single IgSF Ig domain (111-residue C-type for CD79A and 129-residue V-type for CD79B). Each also contains a highly conserved transmembrane domain and a 61 (CD79A) or 48 (CD79B) amino acid cytoplasmic tail that also exhibits striking amino acid evolutionary conservation. CD79A and CD79B are expressed by the earliest committed B-cell progenitors. The CD79A/B heterodimer has also been observed on the surface of early B-cell progenitors in the absence of μ heavy chain, although neither protein is required for progenitors to commit to the B-cell lineage. Later in development, CD79A and CD79B are coexpressed together with Ig of all isotypes on the surface of B cells as a mature BCR complex. The CD79 proteins are specific to the B lineage and are expressed throughout B lymphopoiesis. CD79A and CD79B can be used markers for the identification of B-cell neoplasms, including DLBCL, the majority of acute leukemias of precursor B cell type, in B cell lines, B cell lymphomas, and in some myelomas.

CD20 is also known as Membrane Spanning 4-Domains A (MS4A), Membrane-Spanning 4-Domains, Subfamily A, Member 1, Leukocyte Surface Antigen Leu-16 (LEU-16), B-Lymphocyte Cell-Surface Antigen B1 (B1), S7, and Common Variable Immune Deficiency 5 (CVID5). Illustrative examples of polynucleotide sequences encoding CD79A include, but are not limited to: NM 021950.3, NM_152866.2, AK225630.1, X07203.1, AK292168.1, X12530.1, BC002807.2, NM_023945, NM_152867, ENST00000345732, and ENST00000389939. Illustrative examples of polypeptide sequences encoding CD79a include, but are not limited to: P11836, NX_P11836, ENSP00000432219, ENSP00000433519, ENSP00000432270, ENSP00000314620, ENSP00000437002, ENSP00000374589, ENSP00000433179, ENSP00000433277, NP_690605.1, and NP_068769.2.

CD20 is a member of the membrane-spanning 4A gene family. Members of this nascent protein family are characterized by common structural features and similar intron/exon splice boundaries and display unique expression patterns among hematopoietic cells and nonlymphoid tissues. This CD20 gene encodes a B-lymphocyte surface molecule which plays a role in the development and differentiation of B-cells into plasma cells. CD20 is expressed in a majority of B-cell malignancies, including chronic lymphocytic leukemia, diffuse large B-cell lymphoma, follicular lymphoma, and mantle cell lymphoma.

In various embodiments, immune effector cells modified to express a CAR and a CCR are highly efficacious; undergo robust in vivo expansion; and recognize cancer cells expressing CD79A and/or CD20 and show cytotoxic activity against the CD79A and/or CD20 expressing cancer cells.

In various preferred embodiments, immune effector cells modified to express a CAR and a CCR and further comprise one or more genome edits that reduce or eliminate the expression and/or function of PDCD-1 and/or CBLB.

In one embodiment, an immune effector cell is genetically modified to express an anti-CD79 CAR and an anti-CD20 CCR and further comprises one or more genome edits that decreases or eliminates the expression and function of CBLB. T cells expressing a CAR and CCR are referred to herein as CAR/CCR T cells or CAR/CCR modified T cells.

In one embodiment, a fusion polypeptide comprising an anti-CD79 CAR, a polypeptide cleavage signal and an anti-CD20 CCR is contemplated.

In various embodiments, genetically modified immune effector cells are administered to a subject with cancer cells expressing CD79A and/or CD20 including, but not limited to liquid tumors, hematological malignancies, and B cell malignancies. In one embodiment, immune effector cells modified to express an anti-CD79A CAR and an anti-CD20 CCR are administered to a subject that has DLBCL.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, 9%,±8%,±7%,±6%,±5%,±4%,±3%,±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

As used herein, the terms, “binding domain,” “extracellular domain,” “extracellular binding domain,” “antigen-specific binding domain,” and “extracellular antigen specific binding domain,” are used interchangeably and provide a CAR or CCR with the ability to specifically bind to the target antigen of interest. The binding domain may be derived either from a natural, synthetic, semi-synthetic, or recombinant source.

An “antibody” refers to a binding agent that is a polypeptide comprising at least a light chain or heavy chain immunoglobulin variable region which specifically recognizes and binds an epitope of an antigen, such as a peptide, lipid, polysaccharide, or nucleic acid containing an antigenic determinant, such as those recognized by an immune cell. An “isolated antibody or antigen binding fragment thereof” is one which has been identified and separated and/or recovered from a component of its natural environment.

An “antigen (Ag)” refers to a compound, composition, or substance that can stimulate the production of antibodies or a T cell response in an animal, including compositions (such as one that includes a cancer-specific protein) that are injected or absorbed into an animal. An antigen reacts with the products of specific humoral or cellular immunity, including those induced by heterologous antigens, such as the disclosed antigens. In particular embodiments, the target antigen is an epitope of an CD79A or CD20 polypeptide.

An “epitope” or “antigenic determinant” refers to the region of an antigen to which a binding agent binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation.

As would be understood by the skilled person and as described elsewhere herein, a complete antibody comprises two heavy chains and two light chains. Each heavy chain consists of a variable region and a first, second, and third constant region, while each light chain consists of a variable region and a constant region. Mammalian heavy chains are classified as α, δ, ε, γ, and μ. Mammalian light chains are classified as λ or κ. Immunoglobulins comprising the α, δ, ε, γ, and μ heavy chains are classified as immunoglobulin (Ig)A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge domain for added flexibility; heavy chains μ and ε have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also called “complementarity-determining regions” or “CDRs.” The CDRs can be defined or identified by conventional methods, such as by sequence according to Kabat et al. (Wu, T T and Kabat, E. A., J Exp Med. 132(2):211-50, (1970); Borden, P. and Kabat E. A., PNAS, 84: 2440-2443 (1987); (see, Kabat et al., Sequences of Proteins of Immunological Interest, U.S. Department of Health and Human Services, 1991, which is hereby incorporated by reference), or by structure according to Chothia et al (Chothia, C. and Lesk, A. M., J Mol. Biol., 196(4): 901-917 (1987), Chothia, C. et al, Nature, 342: 877-883 (1989)).

Illustrative examples of rules for predicting light chain CDRs include: CDR-L1 starts at about residue 24, is preceded by a Cys, is about 10-17 residues, and is followed by a Trp (typically Trp-Tyr-Gln, but also, Trp-Leu-Gln, Trp-Phe-Gln, Trp-Tyr-Leu); CDR-L2 starts about 16 residues after the end of CDR-L1, is generally preceded by Ile-Tyr, but also, Val-Tyr, Ile-Lys, Ile-Phe, and is 7 residues; and CDR-L3 starts about 33 residues after the end of CDR-L2, is preceded by a Cys, is 7-11 residues, and is followed by Phe-Gly-XXX-Gly (SEQ ID NO: 97) (XXX is any amino acid).

Illustrative examples of rules for predicting heavy chain CDRs include: CDR-H1 starts at about residue 26, is preceded by Cys-XXX-XXX-XXX (SEQ ID NO: 94), is 10-12 residues and is followed by a Trp (typically Trp-Val, but also, Trp-Ile, Trp-Ala); CDR-H2 starts about 15 residues after the end of CDR-H1, is generally preceded by Leu-Glu-Trp-Ile-Gly (SEQ ID NO: 95), or a number of variations, is 16-19 residues, and is followed by Lys/Arg-Leu/Ile/Val/Phe/Thr/Ala-Thr/Ser/Ile/Ala; and CDR-H3 starts about 33 residues after the end of CDR-H2, is preceded by Cys-XXX-XXX (typically Cys-Ala-Arg), is 3 to 25 residues, and is followed by Trp-Gly-XXX-Gly (SEQ ID NO: 96).

In one embodiment, light chain CDRs and the heavy chain CDRs are determined according to the Kabat method.

In one embodiment, light chain CDRs and the heavy chain CDR2 and CDR3 are determined according to the Kabat method, and heavy chain CDR1 is determined according to the AbM method, which is a comprise between the Kabat and Clothia methods, see e.g., Whitelegg N & Rees A R, Protein Eng. 2000 December; 13(12):819-24 and Methods Mol Biol. 2004; 248:51-91. Programs for predicting CDRs are publicly available, e.g., AbYsis (www.bioinf.org.uk/abysis/).

References to “V_{L}” or “VL” refer to the variable region of an immunoglobulin light chain.

References to “V_{H}” or “VH” refer to the variable region of an immunoglobulin heavy chain.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a mouse. In particular preferred embodiments, a CAR comprises antigen-specific binding domain that is a chimeric antibody or antigen binding fragment thereof.

Human antibodies can be constructed by combining Fv clone variable domain sequence(s) selected from human-derived phage display libraries with known human constant domain sequences(s) as described above. Alternatively, human monoclonal antibodies may be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques andApplications, pp. 51-63 (Marcel Dekker, Inc., New York, 1987); and Boerner et al., J. Immunol., 147: 86 (1991). In addition, transgenic animals (e.g., mice) can be used to produce a full repertoire of human antibodies in the absence of endogenous immunoglobulin production. See, e.g., Jakobovits et al., PNAS USA, 90: 2551 (1993); Jakobovits et al., Nature, 362: 255 (1993); Bruggermann et al., Year in Immunol., 7: 33 (1993). Gene shuffling can also be used to derive human antibodies from non-human, e.g., rodent antibodies, where the human antibody has similar affinities and specificities to the starting non-human antibody. (See PCT WO 93/06213 published Apr. 1, 1993). Unlike traditional humanization of non-human antibodies by CDR grafting, this technique provides completely human antibodies, which have no FR or CDR residues of non-human origin.

A humanized antibody is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. Humanized or other monoclonal antibodies can have additional conservative amino acid substitutions, which have substantially no effect on antigen binding or other immunoglobulin functions. Humanized antibodies can be constructed by means of genetic engineering (see for example, U.S. Pat. No. 5,585,089).

Antibodies include antigen binding fragments thereof, such as Camel Ig, Ig NAR, Fab fragments, Fab′ fragments, F(ab′)2 fragments, bispecific Fab dimers (Fab2), trispecific Fab trimers (Fab3), Fv, single chain Fv proteins (“scFv”), bis-scFv, (scFv)2, minibodies, diabodies, triabodies, tetrabodies, disulfide stabilized Fv proteins (“dsFv”), and single-domain antibody (sdAb, Nanobody) and portions of full length antibodies responsible for antigen binding. The term also includes genetically engineered forms such as chimeric antibodies (for example, humanized murine antibodies), heteroconjugate antibodies (such as, bispecific antibodies) and antigen binding fragments thereof. See also, Pierce Catalog and Handbook, 1994-1995 (Pierce Chemical Co., Rockford, Ill.); Kuby, J., Immunology, 3rd Ed., W. H. Freeman & Co., New York, 1997.

A “heavy chain antibody” refers to an antibody that contains two V_{H} domains and no light chains (Riechmann L. et al, J. Immunol. Methods 231:25-38 (1999); WO94/04678; WO94/25591; U.S. Pat. No. 6,005,079). A “camelid antibody” refers to an antibody isolated from a Camel, Alpaca, or Llama that contains two V_{H} domains and no light chains. A “humanized VHH” or “humanized camelid antibody” refers to a non-human VHH or camelid antibody that has undergone humanization to reduce potential immunogenicity of the antibody in human recipients.

“IgNAR” of “immunoglobulin new antigen receptor” refers to class of antibodies from the shark immune repertoire that consist of homodimers of one variable new antigen receptor (VNAR) domain and five constant new antigen receptor (CNAR) domains. IgNARs represent some of the smallest known immunoglobulin-based protein scaffolds and are highly stable and possess efficient binding characteristics. The inherent stability can be attributed to both (i) the underlying Ig scaffold, which presents a considerable number of charged and hydrophilic surface exposed residues compared to the conventional antibody VH and VL domains found in murine antibodies; and (ii) stabilizing structural features in the complementary determining region (CDR) loops including inter-loop disulphide bridges, and patterns of intra-loop hydrogen bonds.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three hypervariable regions (HVRs) of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge domain. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known. Bispecific Fab dimers (Fab2) have two Fab′ fragments, each binding a different antigen. Trispecific Fab trimers (Fab3) have three Fab′ fragments, each binding a different antigen.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., PNAS USA 90: 6444-6448 (1993). Triabodies and tetrabodies are also described in Hudson et al., Nat. Med. 9:129-134 (2003).

“Single domain antibody” or “sdAb” or “nanobody” refers to an antibody fragment that consists of the variable region of an antibody heavy chain (VH domain) or the variable region of an antibody light chain (VL domain) (Holt, L., et al, Trends in Biotechnology, 21(11): 484-490).

“Single-chain Fv” or “scFv” antibody fragments comprise the VH and VL domains of antibody, wherein these domains are present in a single polypeptide chain and in either orientation (e.g., VL-VH or VH-VL). Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

A “linker” is an amino acid sequence that connect adjacent domains of a polypeptide or fusion polypeptide. A linker sequence includes a “variable region linking sequence,” which is an amino acid sequence that connects the VH and VL domains of an antibody or antigen binding fragment thereof and provides a spacer function compatible with interaction of the two sub-binding domains so that the resulting polypeptide retains a specific binding affinity to the same target molecule as an antibody that comprises the same light and heavy chain variable regions. Illustrative examples of linkers include glycine polymers (G)n; glycine-serine polymers (GI-5S1-5)n, where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). A linker may be 1, 2, 3, 4, 5, 6, 7, 8, 9, 10, 11, 12, 13, 14, 15, 16, 17, 18, 19, 20, 21, 22, 23, 24, 25, or more amino acids long. Other exemplary linkers include, but are not limited to the following amino acid sequences: DGGGS (SEQ ID NO: 57); TGEKP (SEQ ID NO: 58) (see, e.g., Liu et al., PNAS 5525-5530 (1997)); GGRR (SEQ ID NO: 59) (Pomerantz et al. 1995, supra); (GGGGS)n wherein=1, 2, 3, 4 or 5 (SEQ ID NO: 60) (Kim et al., PNAS 93, 1156-1160 (1996.); EGKSSGSGSESKVD (SEQ ID NO: 61) (Chaudhary et al., 1990, Proc. Natl. Acad. Sci. U.S.A. 87:1066-1070); KESGSVSSEQLAQFRSLD (SEQ ID NO: 62) (Bird et al., 1988, Science 242:423-426), GGRRGGGS (SEQ ID NO: 63); LRQRDGERP (SEQ ID NO: 64); LRQKDGGGSERP (SEQ ID NO: 65); LRQKD(GGGS)2 ERP (SEQ ID NO: 66). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods. A linker may comprise the following amino acid sequence: GSTSGSGKPGSGEGSTKG (SEQ ID NO: 67) (Cooper et al., Blood, 101(4): 1637-1644 (2003)).

A “spacer domain,” refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic, or recombinant source. A spacer domain may be a portion of an immunoglobulin, including, but not limited to, one or more heavy chain constant regions, e.g., CH2 and CH3. A spacer domain can include the amino acid sequence of a naturally occurring immunoglobulin hinge domain or an altered immunoglobulin hinge domain.

A “hinge domain,” is a type of spacer domain which plays a role in positioning the antigen binding domain away from the effector cell surface to enable proper cell/cell contact, antigen binding and activation. A hinge domain is placed between the binding domain and the transmembrane domain (TM). The hinge domain may be derived either from a natural, synthetic, semi-synthetic, or recombinant source. The hinge domain can include the amino acid sequence of a naturally occurring immunoglobulin hinge domain or an altered immunoglobulin hinge domain.

An “altered hinge domain” refers to (a) a naturally occurring hinge domain with up to 30% amino acid changes (e.g., up to 25%, 20%, 15%, 10%, or 5% amino acid substitutions or deletions), (b) a portion of a naturally occurring hinge domain that is at least 10 amino acids (e.g., at least 12, 13, 14 or 15 amino acids) in length with up to 30% amino acid changes (e.g., up to 25%, 20%, 15%, 10%, or 5% amino acid substitutions or deletions), or (c) a portion of a naturally occurring hinge domain that comprises the core hinge domain (which may be 4, 5, 6, 7, 8, 9, 10, 11, 12, 13, 14, or 15, or at least 4, 5, 6, 7, 8, 9, 10, 11, 12, 13, 14, or 15 amino acids in length). A hinge domain may be altered by substituting one or more cysteine and/or proline residues in a naturally occurring immunoglobulin hinge domain with one or more other amino acid residues (e.g., one or more serine residues).

A “transmembrane domain” refers to a portion of polypeptide that fuses an extracellular domain to an intracellular domain and anchors the polypeptide to the plasma membrane of the cell. The TM domain may be derived either from a natural, synthetic, semi-synthetic, or recombinant source.

An “intracellular signaling domain,” refers to a polypeptide that participates in transducing the message of effective binding of a target antigen by a receptor expressed on an immune effector cell to into the interior of the immune effector cell to elicit effector cell function, e.g., activation, cytokine production, proliferation and cytotoxic activity, including the release of cytotoxic factors, or other cellular responses elicited with antigen binding to the receptor expressed on the immune effector cell.

The term “effector function” refers to a specialized function of an immune effector cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

It is known that signals generated through the TCR alone are insufficient for full activation of the T cell and that a secondary or costimulatory signal is also required. Thus, T cell activation can be said to be mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal.

A “primary signaling domain” refers to a signaling domain that regulates the primary activation of the TCR complex either in a stimulatory way, or in an inhibitory way. Primary signaling domains that act in a stimulatory manner may contain signaling motifs which are known as immunoreceptor tyrosine-based activation motifs or ITAMs.

As used herein, the term, “costimulatory signaling domain,” or “costimulatory domain”, refers to an intracellular signaling domain of a co-stimulatory molecule. Costimulatory molecules are cell surface molecules other than antigen receptors or Fc receptors that provide a second signal required for efficient activation and function of T lymphocytes upon binding to antigen.

The terms “selectively binds” or “selectively bound” or “selectively binding” or “selectively targets” and describe preferential binding of one molecule to a target molecule (on-target binding) in the presence of a plurality of off-target molecules. In particular embodiments, an HE or megaTAL that targets the PDCD1 gene or CBLB gene selectively binds an on-target DNA binding site about 5, 10, 15, 20, 25, 50, 100, or 1000 times more frequently than an off-target DNA target binding site.

“On-target” refers to a target site sequence.

“Off-target” refers to a sequence similar to but not identical to a target site sequence.

A “target site” or “target sequence” is a chromosomal or extrachromosomal nucleic acid sequence that defines a portion of a nucleic acid to which a binding molecule will bind and/or cleave, provided sufficient conditions for binding and/or cleavage exist. When referring to a polynucleotide sequence or SEQ ID NO. that references only one strand of a target site or target sequence, it would be understood that the target site or target sequence bound and/or cleaved by a nuclease variant is double-stranded and comprises the reference sequence and its complement. In a preferred embodiment, the target site is a sequence in a human PDCD1 gene. In a preferred embodiment, the target site is a sequence in a human CBLB gene.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair (HDR) mechanisms. This process requires nucleotide sequence homology, uses a “donor” molecule as a template to repair a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“NHEJ” or “non-homologous end joining” refers to the resolution of a double-strand break in the absence of a donor repair template or homologous sequence. NHEJ can result in insertions and deletions at the site of the break. NHEJ is mediated by several sub-pathways, each of which has distinct mutational consequences. The classical NHEJ pathway (cNHEJ) requires the KU/DNA-PKcs/Lig4/XRCC4 complex, ligates ends back together with minimal processing and often leads to precise repair of the break. Alternative NHEJ pathways (altNHEJ) also are active in resolving dsDNA breaks, but these pathways are considerably more mutagenic and often result in imprecise repair of the break marked by insertions and deletions. While not wishing to be bound to any particular theory, it is contemplated that modification of dsDNA breaks by end-processing enzymes, such as, for example, exonucleases, e.g., Trex2, may increase the likelihood of imprecise repair.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, polypeptides and nuclease variants, e.g., homing endonuclease variants, megaTALs, etc. contemplated herein are used for targeted double-stranded DNA cleavage. Endonuclease cleavage recognition sites may be on either DNA strand.

An “exogenous” molecule is a molecule that is not normally present in a cell, but that is introduced into a cell by one or more genetic, biochemical or other methods. Exemplary exogenous molecules include, but are not limited to small organic molecules, protein, nucleic acid, carbohydrate, lipid, glycoprotein, lipoprotein, polysaccharide, any modified derivative of the above molecules, or any complex comprising one or more of the above molecules. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, biopolymer nanoparticle, calcium phosphate co-precipitation, DEAE-dextran-mediated transfer and viral vector-mediated transfer.

An “endogenous” molecule is one that is normally present in a particular cell at a particular developmental stage under particular environmental conditions. Additional endogenous molecules can include proteins.

A “gene,” refers to a DNA region encoding a gene product, as well as all DNA regions which regulate the production of the gene product, whether or not such regulatory sequences are adjacent to coding and/or transcribed sequences. A gene includes, but is not limited to, promoter sequences, enhancers, silencers, insulators, boundary elements, terminators, polyadenylation sequences, post-transcription response elements, translational regulatory sequences such as ribosome binding sites and internal ribosome entry sites, replication origins, matrix attachment sites, and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

As used herein, the term “genome editing” refers to the substitution, deletion, and/or introduction of genetic material at a target site in the cell's genome, which restores, corrects, disrupts, and/or modifies expression of a gene or gene product. Genome editing contemplated in particular embodiments comprises introducing one or more nuclease variants, including but not limited to homing endonuclease variants or megaTALs, into a cell to generate DNA lesions at or proximal to a target site in the cell's genome, optionally in the presence of a donor repair template.

Additional definitions are set forth throughout this disclosure.

### C. Chimeric Antigen Receptors

In various embodiments, immune effector cells are modified to express a chimeric antigen receptor (CAR) that targets CD79A and a chimeric costimulatory receptor that targets CD20 in order to redirect cytotoxicity of immune effector cells toward cancer cells expressing CD79A and/or CD20. In various embodiments, immune effector cells are modified to express an anti-CD79 CAR and an anti-CD20 CCR and the cells also have one or more genome edits that reduce the function and expression of CBLB and/or PDCD-1.

Chimeric antigen receptors (CARs) are molecules that combine antibody-based specificity for a desired antigen with a T cell receptor-activating intracellular domain to generate a chimeric protein that exhibits an antigen specific cellular immune activity. As used herein, the term, “chimeric,” describes being composed of parts of different proteins or DNAs from different origins. In particular embodiments, CARs comprise an extracellular domain (also referred to as a binding domain or antigen-specific binding domain) that binds to CD79A, a transmembrane domain, a costimulatory signaling domain, and a primary signaling domain. The main characteristic of CARs is their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors.

In various embodiments, a CAR comprises an extracellular binding domain that comprises a CD79A-specific binding domain; a transmembrane domain; a costimulatory signaling domain and/or a primary signaling domain.

In particular embodiments, a CAR comprises an extracellular binding domain that comprises an anti-CD79A antibody or antigen binding fragment thereof, one or more hinge domains or spacer domains; a transmembrane domain; and a costimulatory signaling domain and/or a primary signaling domain.

In particular embodiments, CARs comprise an extracellular binding domain that comprises an anti-CD79A antibody or antigen binding fragment thereof that specifically binds to a human CD79A polypeptide expressed on a target cell, e.g., a cancer cell.

In particular embodiments, a CD79A-specific binding domain comprises light chain CDR sequences with at least 85%, 86%, 87%, 88%, 89%, 90%, 91%, 92%, 93%, 94%, 95%, 96%, 97%, 98%, or 99% amino acid identity to the light chain CDR sequences set forth in SEQ ID NOs: 1-3 or 9-11. In particular embodiments, a CD79A-specific binding domain comprises light chain CDR sequences set forth in SEQ ID NOs: 1-3 or 9-11.

In a particular embodiment, a CD79A-specific binding domain comprises heavy chain CDR sequences with at least 85%, 86%, 87%, 88%, 89%, 90%, 91%, 92%, 93%, 94%, 95%, 96%, 97%, 98%, or 99% amino acid identity to the heavy chain CDR sequences set forth in SEQ ID NOs: 4-6 or 12-14. In one embodiment, a CD79A-specific binding domain comprises heavy chain CDR sequences set forth in SEQ ID NOs: 4-6 or 12-14.

In particular embodiments, the antigen-specific binding domain is an scFv that binds a human CD79A polypeptide.

In particular embodiments, the antigen-specific binding domain is a humanized camelid VHH that binds a human CD79A polypeptide.

In some embodiments, an anti-CD79A antibody or antigen binding fragment thereof comprises a variable light chain sequence comprising CDRL1-CDRL3 sequences with at least 85%, 86%, 87%, 88%, 89%, 90%, 91%, 92%, 93%, 94%, 95%, 96%, 97%, 98%, or 99% amino acid identity to the amino acid sequences set forth in SEQ ID NOs: 1-3 or 9-11 and/or a variable heavy chain sequence comprising CDRH1-CDRH3 sequences with at least 85%, 86%, 87%, 88%, 89%, 90%, 91%, 92%, 93%, 94%, 95%, 96%, 97%, 98%, or 99% amino acid identity to the amino acid sequence set forth in SEQ ID NOs: 4-6 or 12-14.

In various embodiments, an anti-CD79A antibody or antigen binding fragment thereof comprises a variable light chain sequence comprising CDRL1-CDRL3 sequences set forth in SEQ ID NOs: 1-3 or 9-11 and/or a variable heavy chain sequence comprising CDRH1-CDRH3 sequences set forth in SEQ ID NOs: 4-6 or 12-14.

In preferred embodiments, the anti-CD79A antibody or antigen binding fragment thereof comprises a variable light chain sequence as set forth in any one of SEQ ID NOs: 7 or 15 and/or a variable heavy chain sequence as set forth in any one of SEQ ID NOs: 8 or 16.

In certain embodiments, anti-CD79A CARs comprise linker residues between the various domains, e.g., added for appropriate spacing and conformation of the molecule. In particular embodiments the linker is a variable region linking sequence. Anti-CD79A CARs may comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long. Exemplary linkers include, but are not limited to those encoded by SEQ ID NOs: 57-67.

In particular embodiments, the binding domain of an anti-CD79A CAR is followed by one or more spacer domains. In preferred embodiments, the spacer domain is between the antigen binding domain and the transmembrane domain. In one embodiment, the spacer domain comprises the CH2 and CH3 of IgG1, IgG4, or IgD.

In some embodiments, the binding domain of an anti-CD79A CAR is generally followed by one or more “hinge domains,” which plays a role in positioning the antigen binding domain away from the effector cell surface to enable proper cell/cell contact, antigen binding and activation. Illustrative hinge domains suitable for use in the CARs described herein include the hinge domain derived from the extracellular regions of type 1 membrane proteins such as CD8α, and CD4, which may be wild-type hinge domains from these molecules or may be altered. In one embodiment, the hinge is a PD-1 hinge or CD152 hinge. In a preferred embodiment, the hinge domain comprises a CD8a hinge domain.

In particular embodiments, an anti-CD79A CAR comprises a transmembrane (TM) domain derived from (i.e., comprise at least the transmembrane region(s) of the alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1. In a particular embodiment, the TM domain is synthetic and predominantly comprises hydrophobic residues such as leucine and valine.

In one embodiment an anti-CD79A CAR comprises a TM domain derived from, PD1, CD152, CD28, or CD8α. In another embodiment, an anti-CD79A CAR comprises a TM domain derived from, PD1, CD152, CD28, or CD8a and a short oligo- or polypeptide linker, preferably between 1, 2, 3, 4, 5, 6, 7, 8, 9, or 10 amino acids in length that links the TM domain to an intracellular signaling domain of the CAR. In a preferred embodiment, the TM domain is derived from CD8α.

In particular embodiments, anti-CD79A CARs comprise one or more intracellular signaling domains. In preferred particular embodiments, an anti-CD79A CAR comprises a costimulatory signaling domain and a primary signaling domain. The intracellular primary signaling and costimulatory signaling domains may be linked in any order in tandem to the carboxyl terminus of the transmembrane domain.

In particular embodiments, an anti-CD79A CAR comprises a costimulatory domain isolated from a costimulatory molecule selected from the group consisting of: Toll-like receptor 1 (TLR1), TLR2, TLR3, TLR4, TLR5, TLR6, TLR7, TLR8, TLR9, TLR10, caspase recruitment domain family member 11 (CARD11), CD2, CD7, CD27, CD28, CD30, CD40, CD54 (ICAM), CD83, CD94, CD134 (OX40), CD137 (4-1BB), CD278 (ICOS), DNAX-Activation Protein 10 (DAP10), Linker for activation of T-cells family member 1 (LAT), SH2 Domain-Containing Leukocyte Protein Of 76 kD (SLP76), T cell receptor associated transmembrane adaptor 1 (TRAT1), TNFR2, TNFRS14, TNFRS18, TNRFS25, and zeta chain of T cell receptor associated protein kinase 70 (ZAP70).

In a preferred embodiment, an anti-CD79A CAR comprises a CD28, CD137, or CD134 costimulatory signaling domain.

In particular embodiments, an anti-CD79A CAR comprises an ITAM containing primary signaling domain isolated from a polypeptide selected from the group consisting of FcRγ, FcRβ, CD3γ, CD3δ, CD3ε, CD3ζ, CD22, CD79a, CD79b, and CD66d.

In a preferred embodiment, an anti-CD79A CAR comprises a CD3ζ primary signaling domain.

In particular embodiments, an anti-CD79A CAR comprises an anti-CD79A antibody or antigen binding fragment thereof that specifically binds to a CD79A polypeptide expressed on a cancer cell.

In one embodiment, an anti-CD79A CAR comprises an anti-CD79A antibody or antigen binding fragment that binds a CD79A polypeptide; a transmembrane domain derived from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, AMN1, and PD1; and one or more intracellular costimulatory signaling domains from a costimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD94, CD134 (OX40), CD137 (4-1BB), CD278 (ICOS), DAP10, LAT, SLP76, TRAT1, TNFR2, TNFRS14, TNFRS18, TNRFS25, and ZAP70; and a primary signaling domain from FcRγ, FcRβ, CD3γ, CD3δ, CD3ε, CD3ζ, CD22, CD79a, CD79b, and CD66d.

In one embodiment, an anti-CD79A CAR comprises an anti-CD79A scFv that binds a CD79A polypeptide; a hinge domain selected from the group consisting of: IgG1 hinge/CH2/CH3, IgG4 hinge/CH2/CH3, a PD1 hinge, a CD152 hinge, and a CD8a hinge; a transmembrane domain derived from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, AMN1, and PD1; and one or more intracellular costimulatory signaling domains from a costimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD94, CD134 (OX40), CD137 (4-1BB), CD278 (ICOS), DAP10, LAT, SLP76, TRAT1, TNFR2, TNFRS14, TNFRS18, TNRFS25, and ZAP70; and a primary signaling domain from FcRγ, FcRβ, CD3γ, CD3δ, CD3ε, CD3ζ, CD22, CD79a, CD79b, and CD66d.

In one embodiment, an anti-CD79A CAR comprises an anti-CD79A scFv that binds a CD79A polypeptide; a hinge domain selected from the group consisting of: IgG1 hinge/CH2/CH3, IgG4 hinge/CH2/CH3, a PD1 hinge, a CD152 hinge, and a CD8a hinge; a transmembrane domain derived from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, AMN1, and PD1; a short oligo- or polypeptide linker, preferably between 1, 2, 3, 4, 5, 6, 7, 8, 9, or 10 amino acids in length that links the TM domain to the intracellular signaling domain of the CAR; and one or more intracellular costimulatory signaling domains from a costimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD94, CD134 (OX40), CD137 (4-1BB), CD278 (ICOS), DAP10, LAT, SLP76, TRAT1, TNFR2, TNFRS14, TNFRS18, TNRFS25, and ZAP70; and a primary signaling domain from FcRγ, FcRβ, CD3γ, CD3δ, CD3ε, CD3ζ, CD22, CD79a, CD79b, and CD66d.

In a particular embodiment, an anti-CD79A CAR comprises an anti-CD79A scFv that binds a CD79A polypeptide; a hinge domain comprising an IgG1 hinge/CH2/CH3 polypeptide and a CD8a hinge domain; a CD8α transmembrane domain comprising a polypeptide linker of about 3 to about 10 amino acids; a CD137 intracellular costimulatory signaling domain; and a CD3ζ primary signaling domain.

In a particular embodiment, an anti-CD79A CAR comprises an anti-CD79A scFv that binds a CD79A polypeptide; a CD8a hinge domain; a CD8α transmembrane domain comprising a polypeptide linker of about 3 to about 10 amino acids; a CD134 intracellular costimulatory signaling domain; and a CD3ζ primary signaling domain.

In a particular embodiment, an anti-CD79A CAR comprises an anti-CD79A scFv that binds a CD79A polypeptide; a CD8a hinge domain; a CD8α transmembrane domain comprising a polypeptide linker of about 3 to about 10 amino acids; a CD28 intracellular costimulatory signaling domain; and a CD3ζ primary signaling domain.

In a particular embodiment, an anti-CD79A CAR comprises one or more anti-CD79A VHHs that binds a CD79A polypeptide; a CD8a hinge domain; a CD8α transmembrane domain comprising a polypeptide linker of about 3 to about 10 amino acids; a CD28 intracellular costimulatory signaling domain; and a CD3ζ primary signaling domain.

### D. Chimeric Costimulatory Receptors

In various embodiments, immune effector cells are modified to express an anti-CD79A and an anti-CD20 CCR in order to redirect cytotoxicity of immune effector cells toward cancer cells expressing CD79A and/or CD20 and synergistically increase the effectiveness of the immune effector cell therapy. In various embodiments, immune effector cells are modified to express an anti-CD79A and an anti-CD20 CCR in order to redirect cytotoxicity of immune effector cells toward cancer cells expressing CD79A or CD20 and further comprise one or more gene edits that disrupt or eliminate PDCD-1 and/or CBLB function and/or activity to synergistically increase the effectiveness of the immune effector cell therapy.

Chimeric costimulatory receptors (CCRs) are molecules that combine antibody-based specificity for a desired antigen with a T cell receptor-costimulatory domain but that lacks a primary signaling domain. In particular embodiments, a CCR comprises an extracellular domain (also referred to as a binding domain or antigen-specific binding domain) that binds to CD20, a transmembrane domain, and a costimulatory signaling domain, and lacks a primary signaling domain. The main characteristic of CCRs is their ability to redirect immune effector cell specificity in an MHC independent manner and enhance the immune effector cell response in the presence of a CAR.

In various embodiments, a CCR comprises an extracellular binding domain that comprises a CD20-specific binding domain; a transmembrane domain; and a costimulatory signaling domain, but not a primary signaling domain.

In particular embodiments, a CCR comprises an extracellular binding domain that comprises an anti-CD20 antibody or antigen binding fragment thereof; one or more hinge domains or spacer domains; a transmembrane domain; and a costimulatory signaling domain, but not a primary signaling domain.

In particular embodiments, CCRs comprise an extracellular binding domain that comprises an anti-CD20 antibody or antigen binding fragment thereof that specifically binds to a human CD20 polypeptide expressed on a target cell, e.g., a cancer cell.

In particular embodiments, a CD20-specific binding domain comprises light chain CDR sequences with at least 85%, 86%, 87%, 88%, 89%, 90%, 91%, 92%, 93%, 94%, 95%, 96%, 97%, 98%, or 99% amino acid identity to the light chain CDR sequences set forth in SEQ ID NOs: 25-27. In particular embodiments, an CD20-specific binding domain comprises light chain CDR sequences set forth in SEQ ID NOs: 25-27.

In a particular embodiment, a CD20-specific binding domain comprises heavy chain CDR sequences with at least 85%, 86%, 87%, 88%, 89%, 90%, 91%, 92%, 93%, 94%, 95%, 96%, 97%, 98%, or 99% amino acid identity to the heavy chain CDR sequences set forth in SEQ ID NOs: 28-30. In one embodiment, a CD20-specific binding domain comprises heavy chain CDR sequences set forth in SEQ ID NOs: 28-30.

In particular embodiments, the antigen-specific binding domain is an scFv that binds a human CD20 polypeptide.

In particular embodiments, the antigen-specific binding domain is a humanized camelid VHH that binds a human CD20 polypeptide.

In some embodiments, an anti-CD20 antibody or antigen binding fragment thereof comprises a variable light chain sequence comprising CDRL1-CDRL3 sequences with at least 85%, 86%, 87%, 88%, 89%, 90%, 91%, 92%, 93%, 94%, 95%, 96%, 97%, 98%, or 99% amino acid identity to the amino acid sequences set forth in SEQ ID NOs: 25-27 and/or a variable heavy chain sequence comprising CDRH1-CDRH3 sequences with at least 85%, 86%, 87%, 88%, 89%, 90%, 91%, 92%, 93%, 94%, 95%, 96%, 97%, 98%, or 99% amino acid identity to the amino acid sequence set forth in SEQ ID NOs: 28-30.

In various embodiments, an anti-CD20 antibody or antigen binding fragment thereof comprises a variable light chain sequence comprising CDRL1-CDRL3 sequences set forth in SEQ ID NOs: 25-27 and/or a variable heavy chain sequence comprising CDRH1-CDRH3 sequences set forth in SEQ ID NOs: 28-30.

In preferred embodiments, the anti-CD20 antibody or antigen binding fragment thereof comprises a variable light chain sequence as set forth in SEQ ID NO: 31 and/or a variable heavy chain sequence as set forth in SEQ ID NO: 32.

In certain embodiments, anti-CD20 CCRs comprise linker residues between the various domains, e.g., added for appropriate spacing and conformation of the molecule. In particular embodiments the linker is a variable region linking sequence. Anti-CD20 CCRs may comprise one, two, three, four, or five or more linkers. In particular embodiments, the length of a linker is about 1 to about 25 amino acids, about 5 to about 20 amino acids, or about 10 to about 20 amino acids, or any intervening length of amino acids.

In particular embodiments, the binding domain of an anti-CD20 CCR is followed by one or more spacer domains. In preferred embodiments, the spacer domain is between the antigen binding domain and the transmembrane domain. In one embodiment, the spacer domain comprises the CH2 and CH3 of IgG1, IgG4, or IgD.

In some embodiments, the binding domain of an anti-CD20 CCR is generally followed by one or more “hinge domains,” which plays a role in positioning the antigen binding domain away from the effector cell surface to enable proper cell/cell contact, antigen binding and activation. Illustrative hinge domains suitable for use in the CCRs described herein include the hinge domain derived from the extracellular regions of type 1 membrane proteins such as CD8α, and CD4, which may be wild-type hinge domains from these molecules or may be altered. In one embodiment, the hinge is a PD-1 hinge or CD152 hinge. In a preferred embodiment, the hinge domain comprises a CD8a hinge domain.

In particular embodiments, an anti-CD20 CCR comprises a transmembrane (TM) domain derived from (i.e., comprise at least the transmembrane region(s) of the alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1. In a particular embodiment, the TM domain is synthetic and predominantly comprises hydrophobic residues such as leucine and valine.

In one embodiment an anti-CD20 CCR comprises a TM domain derived from, PD1, CD152, CD28, or CD8α. In another embodiment, an anti-CD20 CCR comprises a TM domain derived from, PD1, CD152, CD28, or CD8a and a short oligo- or polypeptide linker, preferably between 1, 2, 3, 4, 5, 6, 7, 8, 9, or 10 amino acids in length that links the TM domain to an intracellular signaling domain of the CCR. In a preferred embodiment, the TM domain is derived from CD8α.

In particular embodiments, anti-CD20 CCRs comprise one or more intracellular signaling domains. In preferred particular embodiments, an anti-CD20 CCR comprises one or more costimulatory signaling domains but lacks a primary signaling domain. The costimulatory signaling domains may be linked in any order in tandem to the carboxyl terminus of the transmembrane domain.

In particular embodiments, an anti-CD20 CCR comprises a costimulatory domain isolated from a costimulatory molecule selected from the group consisting of: Toll-like receptor 1 (TLR1), TLR2, TLR3, TLR4, TLR5, TLR6, TLR7, TLR8, TLR9, TLR10, caspase recruitment domain family member 11 (CARD11), CD2, CD7, CD27, CD28, CD30, CD40, CD54 (ICAM), CD83, CD94, CD134 (OX40), CD137 (4-1BB), CD278 (ICOS), DNAX-Activation Protein 10 (DAP10), Linker for activation of T-cells family member 1 (LAT), SH2 Domain-Containing Leukocyte Protein Of 76 kD (SLP76), T cell receptor associated transmembrane adaptor 1 (TRAT1), TNFR2, TNFRS14, TNFRS18, TNRFS25, and zeta chain of T cell receptor associated protein kinase 70 (ZAP70).

In a preferred embodiment, an anti-CD79A CAR comprises a CD28, CD137, or CD134 costimulatory signaling domain and an anti-CD20 CCR comprises a different costimulatory domain.

In a preferred embodiment, an anti-CD79A CAR comprises a CD137 or CD134 costimulatory signaling domain and an anti-CD20 CCR comprises a CD28 costimulatory signaling domain.

In a preferred embodiment, an anti-CD79A CAR comprises a CD137 costimulatory signaling domain and an anti-CD20 CCR comprises a CD28 costimulatory signaling domain.

In particular embodiments, an anti-CD20 CCR comprises an anti-CD20 antibody or antigen binding fragment thereof that specifically binds to a CD20 polypeptide expressed on a cancer cell.

In one embodiment, an anti-CD20 CCR comprises an anti-CD20 scFv that binds a CD20 polypeptide; a transmembrane domain derived from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, AMN1, and PD1; and one or more intracellular costimulatory signaling domains from a costimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD94, CD134 (OX40), CD137 (4-1BB), CD278 (ICOS), DAP10, LAT, SLP76, TRAT1, TNFR2, TNFRS14, TNFRS18, TNRFS25, and ZAP70.

In one embodiment, an anti-CD20 CCR comprises an anti-CD20 scFv that binds a CD20 polypeptide; a hinge domain selected from the group consisting of: IgG1 hinge/CH2/CH3, IgG4 hinge/CH2/CH3, a PD1 hinge, a CD152 hinge, and a CD8a hinge; a transmembrane domain derived from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, AMN1, and PD1; a short oligo- or polypeptide linker, preferably between 1, 2, 3, 4, 5, 6, 7, 8, 9, or 10 amino acids in length that links the TM domain to the intracellular signaling domain of the CAR; and one or more intracellular costimulatory signaling domains from a costimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD94, CD134 (OX40), CD137 (4-1BB), CD278 (ICOS), DAP10, LAT, SLP76, TRAT1, TNFR2, TNFRS14, TNFRS18, TNRFS25, and ZAP70.

In a particular embodiment, an anti-CD20 CCR comprises an anti-CD20 scFv that binds a CD20 polypeptide; a hinge domain comprising an IgG1 hinge/CH2/CH3 polypeptide and a CD8a hinge domain; a CD8α transmembrane domain comprising a polypeptide linker of about 3 to about 10 amino acids; and a CD28 intracellular costimulatory signaling domain.

In a particular embodiment, an anti-CD20 CCR comprises an anti-CD20 scFv that binds a CD20 polypeptide; a CD8a hinge domain; a CD8α transmembrane domain comprising a polypeptide linker of about 3 to about 10 amino acids; and a CD28 intracellular costimulatory signaling domain.

### E. Nuclease Variants

In various embodiments, immune effector cells modified to express an anti-CD79A CAR and an anti-CD20 CCR are also genetically modified to reduce or eliminate expression and/or function of PDCD-1 and/or CBLB, using a nuclease variant, such as, for example, a homing endonuclease (meganuclease) variant or megaTAL. Nuclease variants contemplated in particular embodiments herein are suitable for genome editing a target site in a human PDCD-1 gene or a human CBLB gene and comprise one or more DNA binding domains and one or more DNA cleavage domains (e.g., one or more endonuclease and/or exonuclease domains), and optionally, one or more linkers contemplated herein. The terms “reprogrammed nuclease,” “engineered nuclease,” or “nuclease variant” are used interchangeably and refer to a nuclease comprising one or more DNA binding domains and one or more DNA cleavage domains, wherein the nuclease has been designed and/or modified from a parental or naturally occurring nuclease, to bind and cleave a double-stranded DNA target sequence.

In particular embodiments, a nuclease variant binds and cleaves a target sequence in exon 1 of a PDCD-1 gene, preferably at SEQ ID NO: 51 in exon 1 of a PDCD-1 gene, and more preferably at the sequence “ATCC” in SEQ ID NO: 51 in exon 1 of a PDCD-1 gene.

In preferred embodiments, a nuclease variant binds and cleaves a target sequence in exon 6 of a CBLB gene, preferably at SEQ ID NO: 55 in exon 6 of a CBLB gene, and more preferably at the sequence “ATTC” in SEQ ID NO: 55 in exon 6 of a CBLB gene.

The nuclease variant may be designed and/or modified from a naturally occurring nuclease or from a previous nuclease variant. Nuclease variants contemplated in particular embodiments may further comprise one or more additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerases or template-independent DNA polymerase activity.

Illustrative examples of nuclease variants that bind and cleave a target sequence in the a PDCD-1 or CBLB gene include, but are not limited to homing endonuclease (meganuclease) variants and megaTALs.

### 1. Homing Endonuclease (Meganuclease) Variants

In various embodiments, a homing endonuclease or meganuclease is reprogrammed to introduce a double-strand break (DSB) in a target site in a PDCD-1 gene or a CBLB gene. In preferred embodiments, a homing endonuclease or meganuclease is reprogrammed to introduce a double-strand break (DSB) in a target site in a CBLB gene.

In particular embodiments, a homing endonuclease variant introduces a double strand break in exon 1 of a PDCD-1 gene, preferably at SEQ ID NO: 51 in exon 1 of a PDCD-1 gene, and more preferably at the sequence “ATCC” in SEQ ID NO: 51 in exon 1 of a PDCD-1 gene.

In preferred embodiments, a homing endonuclease variant introduces a double strand break in exon 6 of a CBLB gene, preferably at SEQ ID NO: 55 in exon 6 of a CBLB gene, and more preferably at the sequence “ATTC” in SEQ ID NO: 55 in exon 6 of a CBLB gene.

“Homing endonuclease” and “meganuclease” are used interchangeably and refer to naturally-occurring homing endonucleases that recognize 12-45 base-pair cleavage sites and are commonly grouped into five families based on sequence and structure motifs: LAGLIDADG, GIY-YIG, HNH, His-Cys box, and PD-(D/E)XK.

A “reference homing endonuclease” or “reference meganuclease” refers to a wild type homing endonuclease or a homing endonuclease found in nature. In one embodiment, a “reference homing endonuclease” refers to a wild type homing endonuclease that has been modified to increase basal activity.

An “engineered homing endonuclease,” “reprogrammed homing endonuclease,” “homing endonuclease variant,” “engineered meganuclease,” “reprogrammed meganuclease,” or “meganuclease variant” refers to a homing endonuclease comprising one or more DNA binding domains and one or more DNA cleavage domains, wherein the homing endonuclease has been designed and/or modified from a parental or naturally occurring homing endonuclease, to bind and cleave a DNA target sequence. The homing endonuclease variant may be designed and/or modified from a naturally occurring homing endonuclease or from another homing endonuclease variant. Homing endonuclease variants contemplated in particular embodiments may further comprise one or more additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase, template dependent DNA polymerase or template-independent DNA polymerase activity.

Homing endonuclease (HE) variants do not exist in nature and can be obtained by recombinant DNA technology or by random mutagenesis. HE variants may be obtained by making one or more amino acid alterations, e.g., mutating, substituting, adding, or deleting one or more amino acids, in a naturally occurring HE or HE variant. In particular embodiments, a HE variant comprises one or more amino acid alterations to the DNA recognition interface.

In particular embodiments, the homing endonuclease is an I-OnuI HE variant that binds and cleaves a human PDCD-1 gene set forth in SEQ ID NO: 51 comprises an amino acid sequence that is at least 95% identical to the amino acid sequence set forth in SEQ ID NO: 49, or a biologically active fragment thereof.

In preferred embodiments, the homing endonuclease is an I-OnuI HE variant that binds and cleaves a human CBLB gene set forth in SEQ ID NO: 55 comprises an amino acid sequence that is at least 95% identical to the amino acid sequence set forth in SEQ ID NO: 53, or a biologically active fragment thereof.

### 2. Megatals

In various embodiments, a megaTAL comprising a homing endonuclease variant is reprogrammed to introduce a double-strand break (DSB) in a target site in a PDCD-1 gene or a CBLB gene. In preferred embodiments, a megaTAL comprising a homing endonuclease variant is reprogrammed to introduce a double-strand break (DSB) in a target site in a CBLB gene.

In particular embodiments, a megaTAL introduces a DSB in exon 1 of a PDCD-1 gene, preferably at SEQ ID NO: 52 in exon 1 of a PDCD-1 gene, and more preferably at the sequence “ATCC” in SEQ ID NO: 52 in exon 1 of a PDCD-1 gene.

In preferred embodiments, a megaTAL introduces a DSB in exon 6 of a CBLB gene, preferably at SEQ ID NO: 56 in exon 6 of a CBLB gene, and more preferably at the sequence “ATTC” in SEQ ID NO: 56 in exon 6 of a CBLB gene.

A “megaTAL” refers to a polypeptide comprising a TALE DNA binding domain and a homing endonuclease variant that binds and cleaves a DNA target sequence in a gene, and optionally comprises one or more linkers and/or additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase or template-independent DNA polymerase activity.

A “TALE DNA binding domain” is the DNA binding portion of transcription activator-like effectors (TALE or TAL-effectors), which mimics plant transcriptional activators to manipulate the plant transcriptome (see e.g., Kay et al., 2007. Science 318:648-651). TALE DNA binding domains contemplated in particular embodiments are engineered de novo or from naturally occurring TALEs, e.g., AvrBs3 from Xanthomonas campestris pv. vesicatoria, Xanthomonas gardneri, Xanthomonas translucens, Xanthomonas axonopodis, Xanthomonas perforans, Xanthomonas alfalfa, Xanthomonas citri, Xanthomonas euvesicatoria, and Xanthomonas oryzae and brg11 and hpx17 from Ralstonia solanacearum. Illustrative examples of TALE proteins for deriving and designing DNA binding domains are disclosed in U.S. Pat. No. 9,017,967, and references cited therein, all of which are incorporated herein by reference in their entireties.

In particular embodiments, a megaTAL binds and cleaves a target site in the human PDCD-1 gene at SEQ ID NO: 52 and comprises the amino acid sequence set forth in SEQ ID NO: 50.

In preferred embodiments, a megaTAL binds and cleaves a target site in the human CBLB gene at SEQ ID NO: 56 and comprises the amino acid sequence set forth in SEQ ID NO: 54.

### F. Polypeptides

Various polypeptides, fusion polypeptides, and polypeptide variants are contemplated herein, including, but not limited to, CAR polypeptides, CCR polypeptides, CAR-2A-CCR fusion polypeptides, CAR-2A-CAR fusion polypeptides and fragments thereof, homing endonucleases, and megaTALs. In particular embodiments, exemplary polypeptides contemplated herein include polypeptides comprising an amino acid sequence as set forth in any one of SEQ ID NOs: 1-20, 25-33, 35, 37, 39, 41, 43, 45, and 47.

“Polypeptide,” “peptide” and “protein” are used interchangeably, unless specified to the contrary, and according to conventional meaning, i.e., as a sequence of amino acids. Polypeptides are not limited to a specific length, e.g., they may comprise a full-length polypeptide or a polypeptide fragment, and may include one or more post-translational modifications of the polypeptide, for example, glycosylations, acetylations, phosphorylations and the like, as well as other modifications known in the art, both naturally occurring and non-naturally occurring.

An “isolated polypeptide” and the like, as used herein, refer to in vitro synthesis, isolation, and/or purification of a peptide or polypeptide molecule from a cellular environment, and from association with other components of the cell, i.e., it is not significantly associated with in vivo substances. In particular embodiments, an isolated polypeptide is a synthetic polypeptide, a semi-synthetic polypeptide, or a polypeptide obtained or derived from a recombinant source.

Polypeptides include “polypeptide variants.” Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of a CAR and/or CCR by introducing one or more substitutions, deletions, additions and/or insertions into a binding domain, hinge, TM domain, costimulatory signaling domain or primary signaling domain, if present. In particular embodiments, polypeptides include polypeptides having at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% amino acid identity to any of the reference sequences contemplated herein, typically where the variant maintains at least one biological activity of the reference sequence. In particular embodiments, the biological activity is binding affinity. In particular embodiments, the biological activity is cytolytic activity.

Polypeptides include “polypeptide fragments.” Polypeptide fragments refer to a polypeptide, which can be monomeric or multimeric that has an amino-terminal deletion, a carboxyl-terminal deletion, and/or an internal deletion or substitution of a naturally-occurring or recombinantly-produced polypeptide. Illustrative examples of biologically active polypeptide fragments include antibody fragments. As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. Particularly useful polypeptide fragments, in particular embodiments, include functional domains, including antigen-binding domains or fragments of antibodies.

The polypeptide may also be fused in-frame or conjugated to a linker or other sequence for ease of synthesis, purification or identification of the polypeptide (e.g., poly-His), or to enhance binding of the polypeptide to a solid support.

As noted above, in particular embodiments, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Nat. Biomed. Res. Found., Washington, D.C.).

In certain embodiments, a polypeptide variant comprises one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence, e.g., according to Table 1.

TABLE 1
Amino Acid Codons

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |
| Threonine | T | Thr | ACA | ACC | ACG | ACU | | |
| Valine | V | Val | GUA | GUC | GUG | GUU | | |
| Tryptophan | W | Trp | UGG | | | | | |
| Tyrosine | Y | Tyr | UAC | UAU | | | | |

Guidance in determining which amino acid residues can be substituted, inserted, or deleted without abolishing biological activity can be found using computer programs well known in the art, such as DNASTAR, DNA Strider, Geneious, Mac Vector, or Vector NTI software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

In particular embodiments, expression of a CAR and a CCR in the same cell is desired. Polynucleotide sequences encoding a CAR and CCR can be separated by and IRES sequence as discussed elsewhere herein.

In preferred embodiments, fusion polypeptides are contemplated herein.

In a particular preferred embodiment, a CAR and CCR can be expressed as a fusion polypeptide that comprises one or more self-cleaving polypeptide sequences that separate a CAR and CCR.

Fusion polypeptides and fusion proteins refer to a polypeptide having at least two, three, four, five, six, seven, eight, nine, or ten or more polypeptide segments. Fusion polypeptides are typically linked C-terminus to N-terminus, although they can also be linked C-terminus to C-terminus, N-terminus to N-terminus, or N-terminus to C-terminus. The polypeptides of the fusion protein can be in any order or a specified order. In one embodiment, a fusion protein comprises a CAR, a polypeptide cleavage signal, and a CCR. In another embodiment, a fusion protein comprises a CCR, a polypeptide cleavage signal, and a CAR.

In a preferred embodiment, a fusion protein comprises an anti-CD79A CAR, a polypeptide cleavage signal, and an anti-CD20 CCR. In another preferred embodiment, a fusion protein comprises an anti-CD20 CCR, a polypeptide cleavage signal, and an anti-CD79A CAR.

In particular embodiments, a fusion protein comprises an anti-CD79A CAR comprising a variable light chain sequence comprising CDRL1-CDRL3 sequences set forth in SEQ ID NOs: 1-3 or 9-11 and a variable heavy chain sequence comprising CDRH1-CDRH3 sequences set forth in SEQ ID NOs: 4-6 or 12-14; a polypeptide cleavage signal; and an anti-CD20 CCR comprising a variable light chain sequence comprising CDRL1-CDRL3 sequences set forth in SEQ ID NOs: 25-27 and a variable heavy chain sequence comprising CDRH1-CDRH3 sequences set forth in SEQ ID NOs: 28-30.

In particular embodiments, a fusion polypeptide comprises an anti-CD79A CAR comprising a variable light chain sequence as set forth in SEQ ID NO: 7 and/or a variable heavy chain sequence as set forth in SEQ ID NO: 8; a polypeptide cleavage signal; and an anti-CD20 CCR comprising a variable light chain sequence as set forth in SEQ ID NO: 31 and a variable heavy chain sequence as set forth in SEQ ID NO: 32.

In particular embodiments, a fusion protein comprises an anti-CD79A CAR comprising a variable light chain sequence comprising CDRL1-CDRL3 sequences set forth in SEQ ID NOs: 1-3 or 9-11 and a variable heavy chain sequence comprising CDRH1-CDRH3 sequences set forth in SEQ ID NOs: 4-6 or 12-14, a CD8U hinge and transmembrane domain, a CD137 costimulatory domain and a CD3ζ primary signaling domain; a polypeptide cleavage signal; and an anti-CD20 CCR comprising a variable light chain sequence comprising CDRL1-CDRL3 sequences set forth in SEQ ID NOs: 25-27 and a variable heavy chain sequence comprising CDRH1-CDRH3 sequences set forth in SEQ ID NOs: 28-30, a CD8a hinge and transmembrane domain, and a 4-1BB costimulatory domain.

In particular embodiments, a fusion polypeptide comprises an anti-CD79A CAR comprising a variable light chain sequence as set forth in SEQ ID NO: 7 and/or a variable heavy chain sequence as set forth in SEQ ID NO: 8, a CD8a hinge and transmembrane domain, a CD137 costimulatory domain and a CD3ζ primary signaling domain; a polypeptide cleavage signal; and an anti-CD20 CCR comprising a variable light chain sequence as set forth in SEQ ID NO: 31 and a variable heavy chain sequence as set forth in SEQ ID NO: 32, a CD8a hinge domain; a CD8α transmembrane domain, and a 4-1BB costimulatory domain.

Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 68), for example, ENLYFQG (SEQ ID NO: 69) and ENLYFQS (SEQ ID NO: 70), wherein X represents any amino acid (cleavage by TEV occurs between Q and G or Q and S).

In particular embodiments, the polypeptide cleavage signal is a viral self-cleaving peptide or ribosomal skipping sequence.

Illustrative examples of ribosomal skipping sequences include, but are not limited to: a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide.

In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

Illustrative examples of 2A sites are provided in Table 2.

TABLE 2

| | |
|---|---|
| SEQ ID NO: 71 | GSGATNFSLLKQAGDVEENPGP |
| SEQ ID NO: 72 | ATNFSLLKQAGDVEENPGP |
| SEQ ID NO: 73 | LLKQAGDVEENPGP |
| SEQ ID NO: 74 | GSGEGRGSLLTCGDVEENPGP |
| SEQ ID NO: 75 | EGRGSLLTCGDVEENPGP |
| SEQ ID NO: 76 | LLTCGDVEENPGP |
| SEQ ID NO: 77 | GSGQCTNYALLKLAGDVESNPGP |
| SEQ ID NO: 78 | QCTNYALLKLAGDVESNPGP |
| SEQ ID NO: 79 | LLKLAGDVESNPGP |
| SEQ ID NO: 80 | GSGVKQTLNFDLLKLAGDVESNPGP |
| SEQ ID NO: 81 | VKQTLNFDLLKLAGDVESNPGP |
| SEQ ID NO: 82 | LLKLAGDVESNPGP |
| SEQ ID NO: 83 | LLNFDLLKLAGDVESNPGP |
| SEQ ID NO: 84 | TLNFDLLKLAGDVESNPGP |
| SEQ ID NO: 85 | LLKLAGDVESNPGP |
| SEQ ID NO: 86 | NFDLLKLAGDVESNPGP |
| SEQ ID NO: 87 | QLLNFDLLKLAGDVESNPGP |
| SEQ ID NO: 88 | APVKQTLNFDLLKLAGDVESNPGP |
| SEQ ID NO: 89 | VTELLYRMKRAETYCPRPLLAIHPTEARHKQKI |
| | VAPVKQT |
| SEQ ID NO: 90 | LNFDLLKLAGDVESNPGP |
| SEQ ID NO: 91 | LLAIHPTEARHKQKIVAPVKQTLNFDLLKLAGD |
| | VESNPGP |
| SEQ ID NO: 92 | EARHKQKIVAPVKQTLNFDLLKLAGDVESNPGP |

In preferred embodiments, a fusion polypeptide comprises an anti-CD79A CAR comprising the amino acid sequence set forth in any one of SEQ ID NOs: 17-20, a T2A self-cleaving polypeptide, and an anti-CD20 CCR comprising the amino acid sequence set forth in SEQ ID NO: 33 or SEQ ID NO: 35.

In particular preferred embodiments, a fusion polypeptide comprises the amino acid sequence set forth in SEQ ID NO: 37 or SEQ ID NO: 39.

### G. Polynucleotides

In preferred embodiments, a polynucleotide encoding one or more CAR polypeptides, CCR polypeptides, or fusion polypeptide comprising a CAR, 2A peptide, and CCR is provided. As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%,76%, 77%, 78%, 79%, 80%, 81%, 82%, 83%, 84%,85%, 86%, 87%, 88%, 89%, 90%, 91%, 92%, 93%, 94%, 95%, 96%, 97%, 98%, 99% or 100% sequence identity to a reference sequence.

Illustrative examples of polynucleotides include, but are not limited to SEQ ID NOs: 21-24, 34, 36, 38, 40, 42, 44, 46, and 48 and polynucleotides encoding SEQ ID NOs: 1-20, 25-33, 35, 37, 39, 41, 43, 45, and 47.

As used herein, “isolated polynucleotide” refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” also refers to a complementary DNA (cDNA), a recombinant DNA, or other polynucleotide that does not exist in nature and that has been made by the hand of man. In particular embodiments, an isolated polynucleotide is a synthetic polynucleotide, a semi-synthetic polynucleotide, or a polynucleotide obtained or derived from a recombinant source.

In various embodiments, a polynucleotide comprises an mRNA encoding a polypeptide contemplated herein. In certain embodiments, the mRNA comprises a cap, one or more nucleotides, and a poly(A) tail.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, and/or (xi) isolated removal of spurious translation initiation sites.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

Polynucleotide variants include polynucleotide fragments that encode biologically active polypeptide fragments or variants. As used herein, the term “polynucleotide fragment” refers to a polynucleotide fragment at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more nucleotides in length that encodes a polypeptide variant that retains at least 100%, at least 90%, at least 80%, at least 70%, at least 60%, at least 50%, at least 40%, at least 30%, at least 20%, at least 10%, or at least 5% of the naturally occurring polypeptide activity. Polynucleotide fragments refer to a polynucleotide that encodes a polypeptide that has an amino-terminal deletion, a carboxyl-terminal deletion, and/or an internal deletion or substitution of one or more amino acids of a naturally-occurring or recombinantly-produced polypeptide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%8, 85%, 86%, 87%, 88%, 89%, 90%, 91%, 92%, 93%, 94%, 95%, 86%, 97%, 98%, or 99% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene.

Nonetheless, polynucleotides that vary due to differences in codon usage are specifically contemplated in particular embodiments, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment, the nucleic acid cassette encodes an anti-CD79A CAR and an anti-CD20 CCR. The cassette can be removed and inserted into a plasmid or viral vector as a single unit.

Polynucleotides include polynucleotide(s)-of-interest. As used herein, the term “polynucleotide-of-interest” refers to a polynucleotide encoding a polypeptide, polypeptide variant, or fusion polypeptide. A vector may comprise 1, 2, 3, 4, 5, 6, 7, 8, 9, or 10 polynucleotides-of-interest. In certain embodiments, the polynucleotide-of-interest encodes a polypeptide that provides a therapeutic effect in the treatment or prevention of a disease or disorder. Polynucleotides-of-interest, and polypeptides encoded therefrom, include both polynucleotides that encode wild-type polypeptides, as well as functional variants and fragments thereof. In particular embodiments, a functional variant has at least 80%, at least 90%, at least 95%, or at least 99% identity to a corresponding wild-type reference polynucleotide or polypeptide sequence. In certain embodiments, a functional variant or fragment has at least 50%, at least 60%, at least 70%, at least 80%, or at least 90% of a biological activity of a corresponding wild-type polypeptide.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed in particular embodiments, with the total length preferably being limited by the ease of preparation and use in the intended recombinant DNA protocol.

Polynucleotides can be prepared, manipulated and/or expressed using any of a variety of well-established techniques known and available in the art. In order to express a desired polypeptide, a nucleotide sequence encoding the polypeptide, can be inserted into appropriate vector.

Illustrative examples of vectors include, but are not limited to plasmid, autonomously replicating sequences, and transposable elements, e.g., piggyBac, Sleeping Beauty, Mos1, Tcl/mariner, Tol2, mini-Tol2, Tc3, MuA, Himar I, Frog Prince, and derivatives thereof.

Additional Illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include, but are not limited to, pClneo vectors (Promega) for expression in mammalian cells; pLenti4N5-DEST™, pLenti6/5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

The “control elements” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, vectors include, but are not limited to expression vectors and viral vectors, will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGRI), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a R-actin promoter and a myeloproliferative sarcoma virus enhancer, negative control region deleted, dl587rev primer-binding site substituted (MND) U3 promoter (Haas et al. Journal of Virology. 2003; 77(17): 9439-9450).

In one embodiment, a vector comprises an MNDU3 promoter.

In one embodiment, a vector comprises an EF1a promoter comprising the first intron of the human EF1a gene.

In one embodiment, a vector comprises an EF1a promoter that lacks the first intron of the human EF1a gene.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site-specific DNA recombinase. According to certain embodiments the vector comprises at least one (typically two) site(s) for recombination mediated by a site-specific recombinase. As used herein, the terms “recombinase” or “site specific recombinase” include excusive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The vectors may comprise one or more recombination sites for any of a wide variety of site-specific recombinases. It is to be understood that the target site for a site-specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

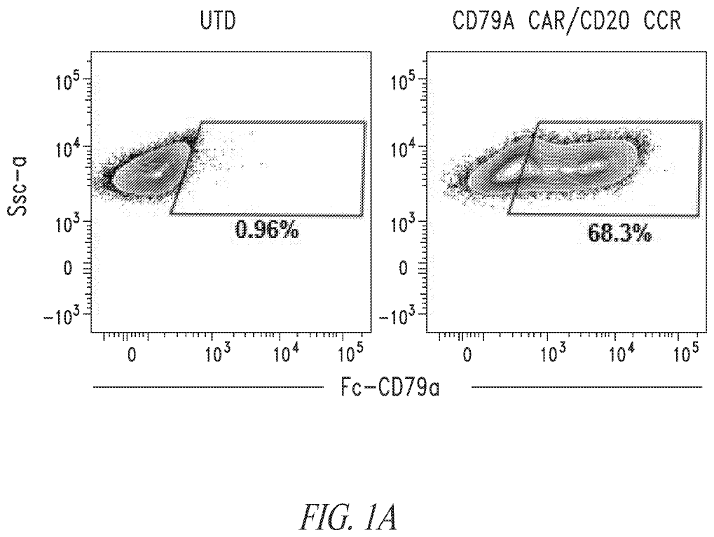

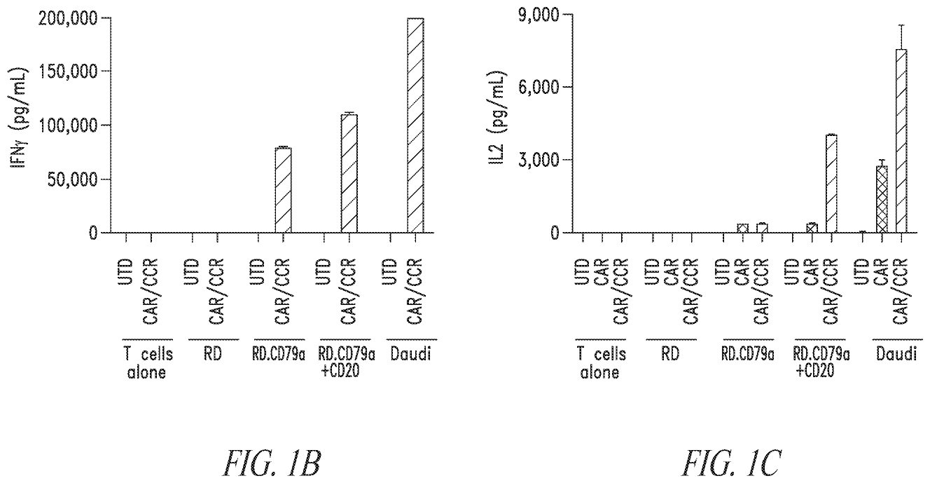

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme λ Integrase, e.g., phi-c31. The φC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by φC31 homodimers (Groth et al., 2000). The product sites, attL and attR, are effectively inert to further φC31-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, vectors include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides. In one embodiment, the IRES used in polynucleotides contemplated herein is an EMCV IRES.

As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO: 93), where R is a purine (A or G) (Kozak, 1986. Cell. 44(2):283-92, and Kozak, 1987. Nucleic Acids Res. 15(20):8125-48). In particular embodiments, the vectors comprise polynucleotides that have a consensus Kozak sequence and that encode a desired polypeptide, e.g., a CAR.

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments, the poly(A) sequence is an SV40 polyA sequence, a bovine growth hormone polyA sequence (BGHpA), a rabbit β-globin polyA sequence (rogpA), variants thereof, or another suitable heterologous or endogenous polyA sequence known in the art.

In some embodiments, a polynucleotide or cell harboring the polynucleotide utilizes a suicide gene, including an inducible suicide gene to reduce the risk of direct toxicity and/or uncontrolled proliferation. In specific aspects, the suicide gene is not immunogenic to the host harboring the polynucleotide or cell. A certain example of a suicide gene that may be used is caspase-9 or caspase-8 or cytosine deaminase. Caspase-9 can be activated using a specific chemical inducer of dimerization (CID).

In particular embodiments, one or more polynucleotides encoding an anti-CD79A CAR and an anti-CD20 CCR are introduced into a cell (e.g., an immune effector cell) by non-viral or viral vectors. In particular embodiments, a polycistronic polynucleotide encoding an anti-CD79A CAR and an anti-CD20 CCR is introduced into a cell by a non-viral or viral vector. In particular embodiments, a polycistronic polynucleotide encoding a fusion protein encoding an anti-CD79A CAR, a 2A self-cleaving polypeptide, and an anti-CD20 CCR is introduced into a cell by a non-viral or viral vector.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to a T cell. In one embodiment, the vector is an in vitro synthesized or synthetically prepared mRNA encoding a polycistronic message encoding an anti-CD79A CAR, and an anti-CD20 CCR.

In one embodiment, the vector is an in vitro synthesized or synthetically prepared mRNA encoding a fusion protein encoding an anti-CD79A CAR, a 2A self-cleaving polypeptide, and an anti-CD20 CCR.

Illustrative examples of non-viral vectors include, but are not limited to mRNA, plasmids (e.g., DNA plasmids or RNA plasmids), transposons, cosmids, and bacterial artificial chromosomes.

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

In various embodiments, the polynucleotide is an mRNA that is introduced into a cell in order to transiently express a desired polypeptide. As used herein, “transient” refers to expression of a non-integrated transgene for a period of hours, days or weeks, wherein the period of time of expression is less than the period of time for expression of the polynucleotide if integrated into the genome or contained within a stable plasmid replicon in the cell.

In particular embodiments, the mRNA encoding a polypeptide is an in vitro transcribed mRNA. As used herein, “in vitro transcribed RNA” refers to RNA, preferably mRNA that has been synthesized in vitro. Generally, the in vitro transcribed RNA is generated from an in vitro transcription vector. The in vitro transcription vector comprises a template that is used to generate the in vitro transcribed RNA.

In particular embodiments, mRNAs may further comprise a comprise a 5′ cap or modified 5′ cap and/or a poly(A) sequence. As used herein, a 5′ cap (also termed an RNA cap, an RNA 7-methylguanosine cap or an RNA m^{7G} cap) is a modified guanine nucleotide that has been added to the “front” or 5′ end of a eukaryotic messenger RNA shortly after the start of transcription. The 5′ cap comprises a terminal group which is linked to the first transcribed nucleotide and recognized by the ribosome and protected from RNases. The capping moiety can be modified to modulate functionality of mRNA such as its stability or efficiency of translation. In a particular embodiment, the mRNA comprises a poly(A) sequence of between about 50 and about 5000 adenines. In one embodiment, the mRNA comprises a poly(A) sequence of between about 100 and about 1000 bases, between about 200 and about 500 bases, or between about 300 and about 400 bases. In one embodiment, the mRNA comprises a poly(A) sequence of about 65 bases, about 100 bases, about 200 bases, about 300 bases, about 400 bases, about 500 bases, about 600 bases, about 700 bases, about 800 bases, about 900 bases, or about 1000 or more bases. poly(A) sequences can be modified chemically or enzymatically to modulate mRNA functionality such as localization, stability or efficiency of translation.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, a viral vector comprising a polynucleotide encoding an anti-CD79A CAR and an anti-CD20 CCR is administered directly to an organism for transduction of cells in vivo. In one embodiment, a viral vector comprising a polynucleotide encoding an anti-CD79A CAR, a 2A self-cleaving polypeptide, and an anti-CD20 CCR is administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated herein include but are not limited to adeno-associated virus (AAV), retrovirus, herpes simplex virus, adenovirus, and vaccinia virus vectors.

In various embodiments, one or more polynucleotides encoding a polycistronic message encoding an anti-CD79A CAR and an anti-CD20 CCR or a fusion protein encoding an anti-CD79A CAR, a 2A self-cleaving polypeptide, and an anti-CD20 CCR are introduced into an immune effector cell, optionally comprising one or more genome edits that reduce or eliminate expression and/or function of PDCD-1 or CBLB, e.g., a T cell, by transducing the cell with a recombinant adeno-associated virus (rAAV), comprising the one or more polynucleotides.

AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2, AAV3, AAV4, AAV5, AAV6, AAV7, AAV8, AAV9, or AAV10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

In various embodiments, one or more polynucleotides encoding a polycistronic message encoding an anti-CD79A CAR and an anti-CD20 CCR or a fusion protein encoding an anti-CD79A CAR, a 2A self-cleaving polypeptide, and an anti-CD20 CCR are introduced into an immune effector cell, optionally comprising one or more genome edits that reduce or eliminate expression and/or function of PDCD-1 or CBLB, by transducing the cell with a retrovirus, e.g., lentivirus, comprising the one or more polynucleotides.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

In various embodiments, a lentiviral vector contemplated herein comprises one or more LTRs, and one or more, or all, of the following accessory elements: a cPPT/FLAP, a Psi (Ψ) packaging signal, an export element, poly (A) sequences, and may optionally comprise a WPRE or HPRE, an insulator element, a selectable marker, and a cell suicide gene, as discussed elsewhere herein.

In particular embodiments, lentiviral vectors contemplated herein may be integrative or non-integrating or integration defective lentivirus. As used herein, the term “integration defective lentivirus” or “IDLV” refers to a lentivirus having an integrase that lacks the capacity to integrate the viral genome into the genome of the host cells. Integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H.

In one embodiment, the HIV-1 integrase deficient pol gene comprises a D64V, Dl161, D116A, E152G, or E152A mutation; D64V, Dl161, and E152G mutations; or D64V, Dl16A, and E152A mutations.

In one embodiment, the HIV-1 integrase deficient pol gene comprises a D64V mutation.

The term “long terminal repeat (LTR)” refers to domains of base pairs located at the ends of retroviral DNAs which, in their natural sequence context, are direct repeats and contain U3, R and U5 regions.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. In another embodiment, a lentiviral vector contains a FLAP element with one or more mutations in the cPPT and/or CTS elements. In yet another embodiment, a lentiviral vector comprises either a cPPT or CTS element. In yet another embodiment, a lentiviral vector does not comprise a cPPT or CTS element.

As used herein, the term “packaging signal” or “packaging sequence” refers to psi [Ψ] sequences located within the retroviral genome which are required for insertion of the viral RNA into the viral capsid or particle, see e.g., Clever et al., 1995. J of Virology, Vol. 69, No. 4; pp. 2101-2109.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J Virol. 65: 1053; and Cullen et al., 1991. Cell 58: 423), and the hepatitis B virus post-transcriptional regulatory element (HPRE).

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766).

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

The terms “pseudotype” or “pseudotyping” as used herein, refer to a virus that has viral envelope proteins that have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells.

In certain embodiments, lentiviral vectors are produced according to known methods. See e.g., Kutner et al., BMC Biotechnol. 2009; 9:10. doi: 10.1186/1472-6750-9-10; Kutner et al. Nat. Protoc. 2009; 4(4):495-505. doi: 10.1038/nprot.2009.22.

According to certain specific embodiments contemplated herein, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides encoding a polycistronic message encoding an anti-CD79A CAR and an anti-CD20 CCR or a fusion protein encoding an anti-CD79A CAR, a 2A self-cleaving polypeptide, and an anti-CD20 CCR are introduced into an immune effector cell, optionally comprising one or more genome edits that reduce or eliminate expression and/or function of PDCD-1 or CBLB, by transducing the cell with an adenovirus comprising the one or more polynucleotides.

Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In various embodiments, one or more polynucleotides encoding a polycistronic message encoding an anti-CD79A CAR and an anti-CD20 CCR or a fusion protein encoding an anti-CD79A CAR, a 2A self-cleaving polypeptide, and an anti-CD20 CCR are introduced into an immune effector cell, optionally comprising one or more genome edits that reduce or eliminate expression and/or function of PDCD-1 or CBLB, by transducing the cell with a herpes simplex virus, e.g., HSV-1, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which are incorporated by reference herein in its entirety.

### H. Genetically Modified Cells

In various embodiments, cells genetically modified to express an anti-CD79A CAR and an anti-CD20 CCR contemplated herein, for use in the treatment of cancer are provided. In various preferred embodiments, immune effector cells genetically modified to express an anti-CD79A CAR and an anti-CD20 CCR and that comprise one or more genome edits that decrease or eliminate the function and/or expression of PDCD-1 and/or CBLB are used in the treatment of cancer. In further preferred embodiments, T cells or NK cells genetically modified to express an anti-CD79A CAR and an anti-CD20 CCR and that comprise one or more genome edits that decrease or eliminate the function and/or expression of CBLB are used in the treatment of cancer.

In particular embodiments, an anti-CD79A CAR and an anti-CD20 CCR or a fusion protein encoding an anti-CD79A CAR, a 2A self-cleaving polypeptide, and an anti-CD20 CCR contemplated herein are introduced and expressed in immune effector cells so as to redirect their specificity to a target antigen of interest, e.g., CD79A or CD20. In preferred embodiments, an anti-CD79A CAR and an anti-CD20 CCR or a fusion protein encoding an anti-CD79A CAR, a 2A self-cleaving polypeptide, and an anti-CD20 CCR are introduced and expressed in immune effector cells comprising one or more genome edits in CBLB so as to redirect their specificity to CD79A and CD20. An “immune effector cell,” is any cell of the immune system that has one or more effector functions (e.g., cytotoxic cell killing activity, secretion of cytokines, induction of ADCC and/or CDC). Illustrative immune effector cells contemplated herein are T lymphocytes, including but not limited to cytotoxic T cells (CTLs; CD8⁺ T cells), TILs, and helper T cells (HTLs; CD4⁺ T cells. In a particular embodiment, the cells comprise αβ T cells. In a particular embodiment, the cells comprise γδ T cells. In one embodiment, immune effector cells include natural killer (NK) cells. In one embodiment, immune effector cells include natural killer T (NKT) cells.

Immune effector cells can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are autologous.

Illustrative immune effector cells used with the anti-CD79A CARs and anti-CD20 CCRs contemplated in particular embodiments include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), CD4⁺CD8⁺ T cell, CD4-CD8⁻ T cell, or any other subset of T cells. Other illustrative populations of T cells suitable for use in particular embodiments include naïve T cells (TN), T memory stem cells (TscM), central memory T cells (TcM), effector memory T cells (TEM), and effector T cells (TEFF).

As would be understood by the skilled person, other cells may also be used as immune effector cells with the anti-CD79A CARs and anti-CD20 CCRs contemplated herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro. Thus, in particular embodiments, immune effector cell includes progenitors of immune effectors cells such as hematopoietic stem cells (HSCs) contained within the CD34⁺ population of cells derived from cord blood, bone marrow or mobilized peripheral blood which upon administration in a subject differentiate into mature immune effector cells, or which can be induced in vitro to differentiate into mature immune effector cells.

The term, “CD34⁺ cell,” as used herein refers to a cell expressing the CD34 protein on its cell surface. “CD34,” as used herein refers to a cell surface glycoprotein (e.g., sialomucin protein) that often acts as a cell-cell adhesion factor and is involved in T cell entrance into lymph nodes. The CD34⁺ cell population contains hematopoietic stem cells (HSC), which upon administration to a patient differentiate and contribute to all hematopoietic lineages, including T cells, NK cells, NKT cells, neutrophils and cells of the monocyte/macrophage lineage.

Methods for making the immune effector cells that express an anti-CD79A CAR and an anti-CD20 CCR contemplated herein are provided in particular embodiments. In one embodiment, the method comprises transfecting or transducing immune effector cells isolated from an individual such that the immune effector cells express a polycistronic message 5 encoding an anti-CD79A CAR and an anti-CD20 CCR or a fusion protein encoding an anti-CD79A CAR, a 2A self-cleaving polypeptide, and an anti-CD20 CCR contemplated herein.

In a preferred embodiment, the method comprises transfecting or transducing immune effector cells isolated from an individual such that the immune effector cells express a polycistronic message encoding an anti-CD79A CAR and an anti-CD20 CCR or a fusion protein encoding an anti-CD79A CAR, a 2A self-cleaving polypeptide, and an anti-CD20 CCR contemplated herein. The method further comprises introducing into the cell, a polynucleotide encoding an HE variant or megaTAL that binds and cleaves a target site in a PDCD-1 gene or CBLB gene, preferably in a CBLB gene. In particular embodiments, the transduced and edited cells are subsequently cultured for expansion, prior to administration to a subject.

In certain embodiments, the immune effector cells are isolated from an individual and genetically modified and/or edited without further manipulation in vitro. Such cells can then be directly re-administered into the individual. In further embodiments, the immune effector cells are first activated and stimulated to proliferate in vitro prior to being genetically modified to express an anti-CD79A CAR and an anti-CD20 CCR and then edited using a HE variant or megaTAL that targets PDCD-1 or CBLB, preferably CBLB. In this regard, the immune effector cells may be cultured before and/or after being genetically modified and/or genome edited (i.e., transduced or transfected to express an anti-CD79A CAR and an anti-CD20 CCR contemplated herein).

In particular embodiments, prior to in vitro manipulation or genetic modification of the immune effector cells described herein, the source of cells is obtained from a subject. In particular embodiments, modified immune effector cells comprise T cells.

In particular embodiments, PBMCs may be directly genetically modified to express a polycistronic message encoding an anti-CD79A CAR and an anti-CD20 CCR or a fusion protein encoding an anti-CD79A CAR, a 2A self-cleaving polypeptide, and an anti-CD20 CCR using methods contemplated herein. In certain embodiments, after isolation of PBMC, T lymphocytes are further isolated and in certain embodiments, both cytotoxic and helper T lymphocytes can be sorted into naïve, memory, and effector T cell subpopulations either before or after genetic modification and/or expansion.

The immune effector cells, such as T cells, can be genetically modified following isolation using known methods, or the immune effector cells can be activated and expanded (or differentiated in the case of progenitors) in vitro prior to being genetically modified and/or genome edited. In a particular embodiment, the immune effector cells, such as T cells, are activated and stimulated for expansion and then genetically modified with the chimeric antigen receptors contemplated herein (e.g., transduced with a viral vector comprising a nucleic acid encoding a polycistronic message encoding an anti-CD79A CAR and an anti-CD20 CCR or a fusion protein encoding an anti-CD79A CAR, a 2A self-cleaving polypeptide, and an anti-CD20 CCR) and then are activated and expanded in vitro. In various embodiments, T cells can be activated and expanded before or after genetic modification, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In one embodiment, CD34⁺ cells are transduced with a nucleic acid construct contemplated herein. In certain embodiments, the transduced CD34⁺ cells differentiate into mature immune effector cells in vivo following administration into a subject, generally the subject from whom the cells were originally isolated. In another embodiment, CD34⁺ cells may be stimulated in vitro prior to exposure to or after being genetically modified with one or more of the following cytokines: Flt-3 ligand (FLT3), stem cell factor (SCF), megakaryocyte growth and differentiation factor (TPO), IL-3 and IL-6 according to the methods described previously (Asheuer et al., 2004; Imren, et al., 2004).

In particular embodiments, a population of modified immune effector cells for the treatment of cancer comprises a CAR and CCR contemplated herein. For example, a population of modified immune effector cells are prepared from peripheral blood mononuclear cells (PBMCs) obtained from a patient diagnosed with B cell malignancy described herein (autologous donors). The PBMCs form a heterogeneous population of T lymphocytes that can be CD4⁺, CD8⁺, or CD4⁺ and CD8⁺.

The PBMCs also can include other cytotoxic lymphocytes such as NK cells or NKT cells. An expression vector carrying the coding sequence of a CAR and CCR contemplated in particular embodiments is introduced into a population of human donor T cells, NK cells or NKT cells. In particular embodiments, successfully transduced T cells that carry the expression vector can be sorted using flow cytometry to isolate CD3 positive T cells and then further propagated to increase the number of these CAR and CCR expressing T cells in addition to cell activation using anti-CD3 antibodies and or anti-CD28 antibodies and IL-2 or any other methods known in the art as described elsewhere herein. Standard procedures are used for cryopreservation of T cells for storage and/or preparation for use in a human subject.

In one embodiment, the in vitro transduction, culture and/or expansion of T cells are performed in the absence of non-human animal derived products such as fetal calf serum and fetal bovine serum. Since a heterogeneous population of PBMCs is genetically modified, the resultant transduced cells are a heterogeneous population of modified cells comprising a polycistronic message encoding an anti-CD79A CAR and an anti-CD20 CCR or a polynucleotide encoding a fusion protein encoding an anti-CD79A CAR, a 2A self-cleaving polypeptide, and an anti-CD20 CCR as contemplated herein. In particular embodiments, a heterogeneous population of PBMCs is genetically modified and genome edited and the resulting cells are a heterogeneous population of modified cells comprising a polycistronic message encoding an anti-CD79A CAR and an anti-CD20 CCR or a polynucleotide encoding a fusion protein encoding an anti-CD79A CAR, a 2A self-cleaving polypeptide, and an anti-CD20 CCR and that further comprise one or more genome edits that decreases or eliminates PDCD-1 and/or CBLB function and expression.

In a further embodiment, a mixture of, e.g., one, two, three, four, five or more, different expression vectors can be used in genetically modifying a donor population of immune effector cells wherein each vector encodes a different chimeric antigen receptor protein as contemplated herein. The resulting modified immune effector cells forms a mixed population of modified cells.

### I. T Cell Manufacturing Methods

In various embodiments, genetically modified T cells are expanded by contact with an agent that stimulates a CD3 TCR complex associated signal and a ligand that stimulates a costimulatory molecule on the surface of the T cells.

In particular embodiments, PBMCs or isolated T cells are contacted with a stimulatory agent and costimulatory agent, such as soluble anti-CD3 and anti-CD28 antibodies, or antibodies attached to a bead or other surface, in a culture medium with appropriate cytokines, such as IL-2, IL-7, and/or IL-15.

In particular embodiments, PBMCs or isolated T cells are contacted with a stimulatory agent and costimulatory agent, such as soluble anti-CD3 and anti-CD28 antibodies, or antibodies attached to a bead or other surface, in a culture medium with appropriate cytokines, such as IL-2, IL-7, and/or IL-15 and/or a PI3K inhibitor.

In one embodiment, peripheral blood mononuclear cells (PBMCs) are used as the source of T cells in the T cell manufacturing methods contemplated herein. PBMCs form a heterogeneous population of T lymphocytes that can be CD4⁺, CD8⁺, or CD4⁺ and CD8⁺ and can include other mononuclear cells such as monocytes, B cells, NK cells and NKT cells. An expression vector comprising a polynucleotide encoding a polycistronic message encoding an anti-CD79A CAR and an anti-CD20 CCR or a fusion protein encoding an anti-CD79A CAR, a 2A self-cleaving polypeptide, and an anti-CD20 CCR contemplated in particular embodiments are introduced into a population of human donor T cells, NK cells or NKT cells. In a particular embodiment, successfully transduced T cells that carry the expression vector can be sorted using flow cytometry to isolate CD3 positive T cells and then further propagated to increase the number of the modified T cells in addition to cell activation using anti-CD3 antibodies and or anti-CD28 antibodies and IL-2, IL-7, and/or IL-15.

In order to achieve sufficient therapeutic doses of T cell compositions, T cells are often subject to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In preferred embodiments, the T cells manufactured by the methods contemplated herein provide improved adoptive immunotherapy compositions. Without wishing to be bound to any particular theory, it is believed that the T cell compositions manufactured by the methods in particular embodiments contemplated herein are imbued with superior properties, including increased survival, expansion in the relative absence of differentiation, and persistence in vivo. In one embodiment, a method of manufacturing T cells comprises contacting the cells with one or more agents that modulate a PI3K cell signaling pathway.

In a particular embodiment, T cells are manufactured by stimulating T cells to become activated and to proliferate in the presence of one or more stimulatory signals and a PI3K inhibitor.

The T cells can then be modified to express a polycistronic message encoding an anti-CD79A CAR and an anti-CD20 CCR or a fusion protein encoding an anti-CD79A CAR, a 2A self-cleaving polypeptide, and an anti-CD20 CCR. In one embodiment, the T cells are modified by transducing the T cells with a viral vector comprising a polycistronic message encoding an anti-CD79A CAR and an anti-CD20 CCR or a fusion protein encoding an anti-CD79A CAR, a 2A self-cleaving polypeptide, and an anti-CD20 CCR contemplated herein. In a certain embodiment, the T cells are modified prior to stimulation and activation in the presence of an inhibitor of a PI3K cell signaling pathway. In another embodiment, T cells are modified after stimulation and activation in the presence of an inhibitor of a PI3K cell signaling pathway. In a particular embodiment, T cells are modified within 12 hours, 24 hours, 36 hours, or 48 hours of stimulation and activation in the presence of an inhibitor of a PI3K cell signaling pathway. In a particular embodiment, the T cells are modified in the presence of a PI3K inhibitor.

In particular embodiments, after the immune effector cells are transduced with a viral vector comprising a polynucleotide encoding an anti-CD79A CAR and an anti-CD20 CCR or a fusion protein encoding an anti-CD79A CAR, a 2A self-cleaving polypeptide, and an anti-CD20 CCR, the cells are edited by introducing a polynucleotide encoding into the cell, a polynucleotide encoding an HE variant or megaTAL that binds and cleaves a target site in a PDCD-1 gene or CBLB gene, preferably in a CBLB gene.

After T cells are transduced and/or edited, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion. In a particular embodiment, the T cells are cultured to proliferate in the presence of a PI3K inhibitor.

In various embodiments, T cell compositions are manufactured in the presence of a PI3K inhibitor. Without wishing to be bound to any particular theory, it is contemplated that treatment or contacting T cells with one or more inhibitors of the PI3K pathway during the stimulation, activation, and/or expansion phases of the manufacturing process preferentially increases young T cells, thereby producing superior therapeutic T cell compositions.

As used herein, the term “PI3K inhibitor” refers to a nucleic acid, peptide, compound, or small organic molecule that binds to and inhibits at least one activity of PI3K. The PI3K proteins can be divided into three classes, class 1 PI3Ks, class 2 PI3Ks, and class 3 PI3Ks. Class 1 PI3Ks exist as heterodimers consisting of one of four p110 catalytic subunits (p110α, p110β, p110δ, and p110γ) and one of two families of regulatory subunits. A PI3K inhibitor preferably targets the class 1 PI3K inhibitors. In one embodiment, a PI3K inhibitor will display selectivity for one or more isoforms of the class 1 PI3K inhibitors (i.e., selectivity for p110α, p110β, p110δ, and p110γ or one or more of p110α, p110β, p110δ, and p110γ). In another aspect, a PI3K inhibitor will not display isoform selectivity and be considered a “pan-PI3K inhibitor.” In one embodiment, a PI3K inhibitor will compete for binding with ATP to the PI3K catalytic domain.

In certain embodiments, a PI3K inhibitor can, for example, target PI3K as well as additional proteins in the PI3K-AKT-mTOR pathway. In particular embodiments, a PI3K inhibitor that targets both mTOR and PI3K can be referred to as either an mTOR inhibitor or a PI3K inhibitor. A PI3K inhibitor that only targets PI3K can be referred to as a selective PI3K inhibitor. In one embodiment, a selective PI3K inhibitor can be understood to refer to an agent that exhibits a 50% inhibitory concentration with respect to PI3K that is at least 10-fold, at least 20-fold, at least 30-fold, at least 50-fold, at least 100-fold, at least 1000-fold, or more, lower than the inhibitor's IC50 with respect to mTOR and/or other proteins in the pathway.

In a particular embodiment, exemplary PI3K inhibitors inhibit PI3K with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one embodiment, a PI3K inhibitor inhibits PI3K with an IC50 from about 2 nM to about 100 nm, more preferably from about 2 nM to about 50 nM, even more preferably from about 2 nM to about 15 nM.

Illustrative examples of PI3K inhibitors suitable for use in the T cell manufacturing methods contemplated in particular embodiments include, but are not limited to, BKM120 (class 1 PI3K inhibitor, Novartis), XL147 (class 1 PI3K inhibitor, Exelixis), (pan-PI3K inhibitor, GlaxoSmithKline), and PX-866 (class 1 PI3K inhibitor; p110α, p110β, and p110γ isoforms, Oncothyreon).

Other illustrative examples of selective PI3K inhibitors include, but are not limited to BYL719, GSK2636771, TGX-221, AS25242, CAL-101, ZSTK474, and IPI-145.

Further illustrative examples of pan-PI3K inhibitors include, but are not limited to BEZ235, LY294002, GSK1059615, TG100713, and GDC-0941.

In a preferred embodiment, the PI3K inhibitor is ZSTK474.

In a particular embodiment, a method for increasing the proliferation of T cells expressing an engineered T cell receptor is provided. Such methods may comprise, for example, harvesting a source of T cells from a subject, stimulating and activating the T cells, modification of the T cells to express an anti-CD79A CAR and an anti-CD20 CCR, editing the cells' genome the with an HE variant or megaTAL and expanding the T cells in culture wherein the T cells are manufactured in the presence of one or more inhibitors of the PI3K pathway.

Manufacturing methods contemplated herein may further comprise cryopreservation of modified T cells for storage and/or preparation for use in a human subject. In one embodiment, a method of storing genetically modified immune effector cells comprises cryopreserving the immune effector cells such that the cells remain viable upon thawing. T cells are cryopreserved such that the cells remain viable upon thawing. When needed, the cryopreserved transformed immune effector cells can be thawed, grown and expanded for more such cells. As used herein, “cryopreserving,” refers to the preservation of cells by cooling to sub-zero temperatures, such as (typically) 77 K or −196° C. (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used include but are not limited to dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidine (Rinfret, Ann. N.Y. Acad. Sci., 1960; 85: 576), and polyethylene glycol (Sloviter and Ravdin, Nature, 1962; 196: 48). The preferred cooling rate is 10 to 3° C./minute. After at least two hours, the T cells have reached a temperature of −80° C. and can be placed directly into liquid nitrogen (−196° C.) for permanent storage such as in a long-term cryogenic storage vessel.

### J. Compositions and Formulations

The compositions contemplated herein may comprise one or more CAR polypeptides, CCR polypeptides, polynucleotides, vectors comprising same, genetically modified immune effector cells, etc., as contemplated herein. Compositions include, but are not limited to pharmaceutical compositions. In preferred embodiments, a composition comprises one or more cells modified to express an anti-CD79A CAR and an anti-CD20 CCR or a fusion protein encoding an anti-CD79A CAR, a 2A self-cleaving polypeptide, and an anti-CD20 CCR. In preferred embodiments, a composition comprises one or more cells modified to express an anti-CD79A CAR and an anti-CD20 CCR or a fusion protein encoding an anti-CD79A CAR, a 2A self-cleaving polypeptide, and an anti-CD20 CCR, wherein the cells have also undergone genome editing to reduce or eliminate expression and function of CBLB.

A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy. In preferred embodiments, a pharmaceutical composition comprises a pharmaceutically acceptable carrier, diluent or excipient and one or more genome edited cells that have also been modified to express an anti-CD79A CAR and an anti-CD20 CCR or a fusion protein encoding an anti-CD79A CAR, a 2A self-cleaving polypeptide, and an anti-CD20 CCR.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions comprise an amount of anti-CD79A CAR and anti-CD20 CCR-expressing immune effector cells contemplated herein. In preferred embodiments, compositions comprise an amount of genome edited immune effector cells that express an anti-CD79A CAR and an anti-CD20 CCR, wherein the genome edit(s) reduce or eliminate expression and function of PDCD-1 and/or CBLB, preferably CBLB. As used herein, the term “amount” refers to “an amount effective” or “an effective amount” of a genetically modified therapeutic cell, e.g., T cell, to achieve a beneficial or desired prophylactic or therapeutic result, including clinical results.

A “prophylactically effective amount” refers to an amount of a genetically modified therapeutic cells effective to achieve the desired prophylactic result. Typically, but not necessarily, since a prophylactic dose is used in subjects prior to or at an earlier stage of disease, the prophylactically effective amount is less than the therapeutically effective amount.

A “therapeutically effective amount” of a genetically modified therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). When a therapeutic amount is indicated, the precise amount of the compositions to be administered can be determined by a physician with consideration of individual differences in age, weight, tumor size, extent of infection or metastasis, and condition of the patient (subject).

It can generally be stated that a pharmaceutical composition comprising the T cells described herein may be administered at a dosage of 102 to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mLs or less, even 250 mLs or 100 mLs or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects, particularly since all the infused cells will be redirected to a particular target antigen, lower numbers of cells, in the range of 10⁶/kilogram (10⁶-10¹¹ per patient) may be administered. Compositions may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-12, TNF-alpha, IL-18, and TNF-beta, GM-CSF, IL-4, IL-13, Flt3-L, RANTES, MIPlu, etc.) as described herein to enhance induction of the immune response.

Generally, compositions comprising the cells activated and expanded as described herein may be utilized in the treatment and prevention of diseases that arise in individuals who are immunocompromised. In particular embodiments, compositions comprising immune effector cells modified to express an anti-CD79A CAR and an anti-CD20 CCR or a fusion protein encoding an anti-CD79A CAR, a 2A self-cleaving polypeptide, and an anti-CD20 CCR contemplated herein and comprising one or more genome edits that reduce or eliminate expression and function of PDCD-1 and/or CBLB, preferably CBLB, are used in the treatment of cancer. The modified immune effector cells may be administered either alone, or as a pharmaceutical composition in combination with carriers, diluents, excipients, and/or with other components such as IL-2 or other cytokines or cell populations. In particular embodiments, pharmaceutical compositions comprise an amount of genetically modified T cells, in combination with one or more pharmaceutically or physiologically acceptable carriers, diluents or excipients.

Pharmaceutical compositions comprising a genome edited immune effector cell population modified to express an anti-CD79A CAR and an anti-CD20 CCR or a fusion protein encoding an anti-CD79A CAR, a 2A self-cleaving polypeptide, and an anti-CD20 CCR, such as T cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions are preferably formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In one embodiment, the T cell compositions contemplated herein are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to QBSF-60 (Quality Biological, Inc.), StemPro-34 (Life Technologies), and X-VIVO 10.

In one preferred embodiment, compositions comprising immune effector cells contemplated herein are formulated in a solution comprising PlasmaLyte A.

In another preferred embodiment, compositions comprising immune effector cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In a more preferred embodiment, compositions comprising immune effector cells contemplated herein are formulated in a solution comprising 50:50 PlasmaLyte A to CryoStor CS10.

In a particular embodiment, compositions comprise an effective amount of genome edited immune effector cells modified to express an anti-CD79A CAR and an anti-CD20 CCR or a fusion protein encoding an anti-CD79A CAR, a 2A self-cleaving polypeptide, and an anti-CD20 CCR, alone or in combination with one or more therapeutic agents. Thus, the CAR-expressing immune effector cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated in particular embodiments include cytokines, growth factors, steroids, NSAIDs, DMARDs, anti-inflammatories, chemotherapeutics, radiotherapeutics, therapeutic antibodies, or other active and ancillary agents.

In certain embodiments, compositions comprising genome edited immune effector cells modified to express an anti-CD79A CAR and an anti-CD20 CCR or a fusion protein encoding an anti-CD79A CAR, a 2A self-cleaving polypeptide, and an anti-CD20 CCR may be administered in conjunction with any number of chemotherapeutic agents.

A variety of other therapeutic agents may be used in conjunction with the compositions described herein. In one embodiment, the composition comprising genome edited immune effector cells an anti-CD79A CAR and an anti-CD20 CCR or a fusion protein encoding an anti-CD79A CAR, a 2A self-cleaving polypeptide, and an anti-CD20 CCR is administered with an anti-inflammatory agent.

In one embodiment, the composition comprising genome edited immune effector cells an anti-CD79A CAR and an anti-CD20 CCR or a fusion protein encoding an anti-CD79A CAR, a 2A self-cleaving polypeptide, and an anti-CD20 CCR is administered with a therapeutic antibody. Illustrative examples of therapeutic antibodies suitable for combination with the CAR modified T cells contemplated in particular embodiments, include but are not limited to, atezolizumab, avelumab, bavituximab, bevacizumab (avastin), bivatuzumab, blinatumomab, conatumumab, crizotinib, daratumumab, duligotumab, dacetuzumab, dalotuzumab, durvalumab, elotuzumab (HuLuc63), gemtuzumab, ibritumomab, indatuximab, inotuzumab, ipilimumab, lorvotuzumab, lucatumumab, milatuzumab, moxetumomab, nivolumab, ocaratuzumab, ofatumumab, pembrolizumab, rituximab, siltuximab, teprotumumab, and ublituximab.

### K. Targets Cells and Antigens

Genetically modified immune effector cells redirected to a target cell, e.g., cancer cell, and that express an anti-CD79A CAR and an anti-CD20 CCR and that comprise one or more genome edits in PDCD-1 and/or CBLB, preferably CBLB, are provided in particular embodiments. As used herein, the term “cancer” relates generally to a class of diseases or conditions in which abnormal cells divide without control and can invade nearby tissues

As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “benign” or “non-malignant” refers to tumors that may grow larger but do not spread to other parts of the body. Benign tumors are self-limited and typically do not invade or metastasize.

A “cancer cell” refers to an individual cell of a cancerous growth or tissue. Cancer cells include both solid cancers and liquid cancers. A “tumor” or “tumor cell” refers generally to a swelling or lesion formed by an abnormal growth of cells, which may be benign, pre-malignant, or malignant. Most cancers form tumors, but liquid cancers, e.g., leukemia, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

The term “relapse” refers to the diagnosis of return, or signs and symptoms of return, of a cancer after a period of improvement or remission.

“Remission,” is also referred to as “clinical remission,” and includes both partial and complete remission. In partial remission, some, but not all, signs and symptoms of cancer have disappeared. In complete remission, all signs and symptoms of cancer have disappeared, although cancer still may be in the body.

“Refractory” refers to a cancer that is resistant to, or non-responsive to, therapy with a particular therapeutic agent. A cancer can be refractory from the onset of treatment (i.e., non-responsive to initial exposure to the therapeutic agent), or as a result of developing resistance to the therapeutic agent, either over the course of a first treatment period or during a subsequent treatment period.

In one embodiment, the target cell expresses an antigen, e.g., a target antigen that is not substantially found on the surface of other normal (desired) cells.

In one embodiment, the target cell is an osteosarcoma cell or a Ewing's sarcoma cell.

In one embodiment, the target cell is a hematopoietic cell, a lymphoid cell, or a myeloid cell.

In certain embodiments, the target cell is part of the blood, a lymphoid tissue, or a myeloid tissue.

In a particular embodiment, the target cell is a cancer cell or cancer stem cell that expresses CD79A and/or CD20. In a particular embodiment, the target cell is a cancer cell or cancer stem cell that expresses CD79A and CD20. In a particular embodiment, the target cell is a cancer cell or cancer stem cell that expresses CD79A or CD20.

In a particular embodiment, the target cell is a liquid cancer cell or hematological cancer cell that expresses CD79A and/or CD20. In a particular embodiment, the target cell is a liquid cancer cell or hematological cancer cell that expresses CD79A and CD20. In a particular embodiment, the target cell is a liquid cancer cell or hematological cancer cell that expresses CD79A or CD20.

Illustrative examples of liquid cancers or hematological cancers that may be prevented, treated, or ameliorated with the compositions contemplated in particular embodiments include, but are not limited to: leukemias, lymphomas, and multiple myeloma.

Illustrative examples of cells that can be targeted by immune effector cells expressing an anti-CD79A CAR and an anti-CD20 CCR contemplated in particular embodiments include, but are not limited to those of the following leukemias: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera.

Illustrative examples of cells that can be targeted by the compositions comprising immune effector cells expressing an anti-CD79A CAR and an anti-CD20 CCR and methods contemplated in particular embodiments include, but are not limited to those of the following lymphomas: Hodgkin lymphoma, nodular lymphocyte-predominant Hodgkin lymphoma and Non-Hodgkin lymphoma, including but not limited to B-cell non-Hodgkin lymphomas: Burkitt lymphoma, small lymphocytic lymphoma (SLL), diffuse large B-cell lymphoma (DLBCL), follicular lymphoma, immunoblastic large cell lymphoma, precursor B-lymphoblastic lymphoma, and mantle cell lymphoma; and T-cell non-Hodgkin lymphomas: mycosis fungoides, anaplastic large cell lymphoma, Sezary syndrome, and precursor T-lymphoblastic lymphoma.

Illustrative examples of cells that can be targeted by the compositions comprising immune effector cells expressing an anti-CD79A CAR and an anti-CD20 CCR and methods contemplated in particular embodiments include, but are not limited to those of the following multiple myelomas: overt multiple myeloma, smoldering multiple myeloma (MGUS), plasma cell leukemia, non-secretory myeloma, IgD myeloma, osteosclerotic myeloma, solitary plasmacytoma of bone, and extramedullary plasmacytoma.

In preferred embodiments, the CD79A and/or CD20 expressing target cell is a DLBCL cancer cell. In preferred embodiments, the CD79A and CD20 expressing target cell is a DLBCL cancer cell. In preferred embodiments, the CD79A or CD20 expressing target cell is a DLBCL cancer cell.

### L. Therapeutic Methods

The genetically modified immune effector cells expressing an anti-CD79A CAR and an anti-CD20 CCR contemplated herein provide improved methods of adoptive immunotherapy for use in the prevention, treatment, and amelioration cancers that express CD79A and/or CD20 or for preventing, treating, or ameliorating at least one symptom associated with an CD79A and/or CD20 expressing cancer.

In various embodiments, the genetically modified immune effector cells contemplated herein provide improved methods of adoptive immunotherapy for use in increasing the cytotoxicity in cancer cells that express CD79A and/or CD20 in a subject or for use in decreasing the number of cancer cells expressing CD79A and/or CD20 in a subject.

In particular embodiments, the specificity of a primary immune effector cell is redirected to cells expressing CD79A and/or CD20, e.g., cancer cells, by genetically modifying the primary immune effector cell with a CAR contemplated herein. In various embodiments, a viral vector is used to genetically modify an immune effector cell with a particular polynucleotide encoding an anti-CD79A CAR and an anti-CD20 CCR.

In one embodiment, a type of cellular therapy where T cells are genetically modified to express an anti-CD79A CAR and an anti-CD20 CCR that targets CD79A and/or CD20 expressing cancer cells, and the T cells are infused to a recipient in need thereof is provided. The infused cell is able to kill disease causing cells in the recipient. Unlike antibody therapies, T cell therapies are able to replicate in vivo resulting in long-term persistence that can lead to sustained cancer therapy.

In one embodiment, T cells that express an anti-CD79A CAR and an anti-CD20 CCR can undergo robust in vivo T cell expansion and can persist for an extended amount of time. In another embodiment, T cells that express an anti-CD79A CAR and an anti-CD20 CCR evolve into specific memory T cells or stem cell memory T cells that can be reactivated to inhibit any additional tumor formation or growth.

In particular embodiments, compositions comprising immune effector cells that express an anti-CD79A CAR and an anti-CD20 CCR contemplated herein are used in the treatment of conditions associated with CD79A and/or CD20 expressing cancer cells or cancer stem cells.

Illustrative examples of conditions that can be treated, prevented or ameliorated using the immune effector cells that express an anti-CD79A CAR and an anti-CD20 CCR contemplated in particular embodiments

In a particular embodiment, compositions comprising T cells that express an anti-CD79A CAR and an anti-CD20 CCR contemplated herein are used in the treatment of osteosarcoma or Ewing's sarcoma.

In a particular embodiment, compositions comprising T cells that express an anti-CD79A CAR and an anti-CD20 CCR contemplated herein are used in the treatment of liquid or hematological cancers.

In certain embodiments, the liquid or hematological cancer is selected from the group consisting of: leukemias, lymphomas, and multiple myelomas.

In certain embodiments, the liquid or hematological cancer is selected from the group consisting of: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sezary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In certain embodiments, the liquid or hematological cancer is selected from the group consisting of: acute lymphocytic leukemia (ALL), chronic lymphocytic leukemia (CLL), hairy cell leukemia (HCL), multiple myeloma (MM), acute myeloid leukemia (AML), or chronic myeloid leukemia (CML).

In preferred embodiments, the liquid or hematological cancer is DLBCL.

In preferred embodiments, the liquid or hematological cancer is relapsed/refractory DLBCL.

In particular embodiments, methods comprising administering a therapeutically effective amount of immune effector cells that express an anti-CD79A CAR and an anti-CD20 CCR contemplated herein or a composition comprising the same, to a patient in need thereof, alone or in combination with one or more therapeutic agents, are provided. In certain embodiments, the cells are used in the treatment of patients at risk for developing a condition associated with cancer cells that express CD79A and/or CD20. Thus, in particular embodiments, methods for the treatment or prevention or amelioration of at least one symptom of cancer comprising administering to a subject in need thereof, a therapeutically effective amount of the modified T cells that express an anti-CD79A CAR and an anti-CD20 CCR contemplated herein.

As used herein, the terms “individual” and “subject” are often used interchangeably and refer to any animal that exhibits a symptom of a disease, disorder, or condition that can be treated with the gene therapy vectors, cell-based therapeutics, and methods contemplated elsewhere herein. In preferred embodiments, a subject includes any animal that exhibits symptoms of a disease, disorder, or condition related to cancer that can be treated with the gene therapy vectors, cell-based therapeutics, and methods contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have a CD79A and/or CD20 expressing cancer, have been diagnosed with a CD79A and/or CD20 expressing cancer, or are at risk or having a CD79A and/or CD20 expressing cancer, e.g., DLBCL.

As used herein, the term “patient” refers to a subject that has been diagnosed with a particular disease, disorder, or condition that can be treated with the gene therapy vectors, cell-based therapeutics, and methods disclosed elsewhere herein.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the phrase “ameliorating at least one symptom of” refers to decreasing one or more symptoms of the disease or condition for which the subject is being treated. In particular embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

By “enhance” or “promote,” or “increase” or “expand” refers generally to the ability of a composition contemplated herein, e.g., a genetically modified T cells that express an anti-CD79A CAR and an anti-CD20 CCR, to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a similar physiological response (i.e., downstream effects) in a cell, as compared to the response caused by either vehicle, a control molecule/composition, or the response in a particular cell lineage. A comparable response is one that is not significantly different or measurable different from the reference response.

In one embodiment, a method of treating cancer in a subject in need thereof comprises administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

In one embodiment, the amount of immune effector cells, e.g., T cells that express an anti-CD79A CAR and an anti-CD20 CCR, in the composition administered to a subject is at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2×10⁹ cells, at least 3×10⁹ cells, at least 4×10⁹ cells, at least 5×10⁹ cells, or at least 1×10¹⁰ cells.

In particular embodiments, about 1×10⁷ T cells to about 1×10⁹ T cells, about 2×10⁷ T cells to about 0.9×10⁹ T cells, about 3×10⁷ T cells to about 0.8×10⁹ T cells, about 4×10⁷ T cells to about 0.7×10⁹ T cells, about 5×10⁷ T cells to about 0.6×10⁹ T cells, or about 5×10⁷ T cells to about 0.5×10⁹ T cells are administered to a subject.

In one embodiment, the amount of immune effector cells, e.g., T cells that express an anti-CD79A CAR and an anti-CD20 CCR, in the composition administered to a subject is at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight.

In particular embodiments, about 1×10⁶ T cells/kg of bodyweight to about 1×10⁸ T cells/kg of bodyweight, about 2×10⁶ T cells/kg of bodyweight to about 0.9×10⁸ T cells/kg of bodyweight, about 3×10⁶ T cells/kg of bodyweight to about 0.8×10⁸ T cells/kg of bodyweight, about 4×10⁶ T cells/kg of bodyweight to about 0.7×10⁸ T cells/kg of bodyweight, about 5×10⁶ T cells/kg of bodyweight to about 0.6×10⁸ T cells/kg of bodyweight, or about 5×10⁶ T cells/kg of bodyweight to about 0.5×10⁸ T cells/kg of bodyweight are administered to a subject.

One of ordinary skill in the art would recognize that multiple administrations of the compositions contemplated herein may be required to effect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In certain embodiments, it may be desirable to administer activated immune effector cells to a subject and then subsequently redraw blood (or have an apheresis performed), activate immune effector cells therefrom, and reinfuse the patient with these activated and expanded immune effector cells. This process can be carried out multiple times every few weeks. In certain embodiments, immune effector cells can be activated from blood draws of from 10 cc to 400 cc. In certain embodiments, immune effector cells are activated from blood draws of 20 cc, 30 cc, 40 cc, 50 cc, 60 cc, 70 cc, 80 cc, 90 cc, 100 cc, 150 cc, 200 cc, 250 cc, 300 cc, 350 cc, or 400 cc or more. Not to be bound by theory, using this multiple blood draw/multiple reinfusion protocol may serve to select out certain populations of immune effector cells.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

In one embodiment, a subject in need thereof is administered an effective amount of a composition to increase a cellular immune response to a B cell related condition in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

In one embodiment, a method of treating a subject diagnosed with a CD79A and/or CD20 expressing cancer is provided comprising removing immune effector cells from the subject, genetically modifying said immune effector cells with a vector comprising a nucleic acid encoding an anti-CD79A CAR and an anti-CD20 CCR contemplated herein, thereby producing a population of modified immune effector cells, and administering the population of modified immune effector cells to the same subject. In a preferred embodiment, the immune effector cells comprise T cells.

In certain embodiments, methods for stimulating an immune effector cell mediated immune modulator response to a target cell population in a subject are provided comprising the steps of administering to the subject an immune effector cell population expressing a nucleic acid construct encoding an anti-CD79A CAR and an anti-CD20 CCR.

The methods for administering the cell compositions contemplated in particular embodiments includes any method which is effective to result in reintroduction of ex vivo genetically modified immune effector cells that either directly express an anti-CD79A CAR and an anti-CD20 CCR in the subject or on reintroduction of the genetically modified progenitors of immune effector cells that on introduction into a subject differentiate into mature immune effector cells that express the anti-CD79A CAR and anti-CD20 CCR. One method comprises transducing peripheral blood T cells ex vivo with a nucleic acid construct contemplated herein and returning the transduced cells into the subject.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Construction of Anti-CD79A CAR-Anti-CD20 CCR Bicistronic Constructs

Lentiviral vectors comprising bicistronic constructs that include a humanized anti-CD79A CAR, a T2A self-cleaving polypeptide, and an anti-CD20 CCR were designed, constructed, and verified. Constructs comprising an MNDU3 promoter operably linked to an anti-CD79A CAR that contains a CD8α signal sequence, an anti-CD79A scFv, a CD8a hinge and transmembrane domain, a CD137 costimulatory domain, and a CD3ζ primary signaling domain; a T2A self-cleaving polypeptide; and an anti-CD20 CCR that contains an anti-CD20scFv, a CD8a hinge and transmembrane domain, a CD28 costimulatory domain, were cloned into lentiviral vectors. Exemplary anti-CD79A CAR/T2A/anti-CD20 CCR polypeptide sequences are set forth in SEQ ID NOs: 37 and 39 and exemplary anti-CD79A CAR/T2A/anti-CD20 CCR polynucleotide sequences are set forth in SEQ ID NOs: 38 and 40.

### Example 2

### T Cells Expressing Both an Anti-CD79A CAR and an Anti-CD20 CCR Show Antigen Dependent Cytokine Release

Peripheral blood mononuclear cells (PBMCs) were harvested from healthy human donors and activated using anti-CD3 and anti-CD28 antibodies. Activated cells were transduced with a lentiviral vector comprising a polynucleotide encoding an anti-CD79A 41BB CD3ζ CAR (SEQ ID NO: 18) and an anti-CD20 CCR (SEQ ID NO: 33). The polynucleotide is expressed as a fusion protein (SEQ ID NO: 37) wherein the anti-CD79A CAR and the anti-CD20 CCR are separated by a T2A viral self-cleaving polypeptide. Untransduced (UTD) cells were used as a control. After transduction, the cells were expanded in T cell growth medium containing IL-2 for 10 days.

After expansion, transduced and untransduced T cell cultures were evaluated by flow cytometry for anti-CD79A CAR expression using a recombinant Fc-CD79A fusion protein conjugated to the PE fluorochrome. High expression was observed in the T cells transduced with the lentiviral vector encoding the anti-CD79A CAR compared to untransduced control T cells. FIG. 1A.

Anti-CD79A CAR-anti-CD20 CCR T cell functionality was also evaluated. Untransduced T cells or T cells transduced with lentiviral vectors encoding an anti-CD79A CAR (SEQ ID NO: 18) or encoding an anti-CD79A 41BB CD3ζ CAR, T2A, anti-CD20 CCR fusion protein (SEQ ID NO: 37) were cultured alone or co-cultured at a 1:1 ratio with tumor cells lacking target antigen (RD; Rhabdosarcoma cell line); and with RD.79A cells (RD modified to express CD79A), RD.CD79A.CD20 cells (RD modified to express CD79A and CD20), and Daudi cells (Burkitt's lymphoma, which have high endogenous expression of both CD79a and CD20). Following 24 hours of co-culture, supernatants were collected and analyzed for IFNγ and IL-2 cytokines using a Luminex assay. Anti-CD79A CAR-anti-CD20 CCR T cells produced IFNγ cytokine in response to only the cell lines expressing CD79A, but not against RD cells. FIG. 1B. IL-2 production was increased in anti-CD79A CAR-anti-CD20 CCR T cells compared to anti-CD79A CAR T cells because the activity of the anti-CD20 CCR through its CD28 costimulatory domain induces the PI3K pathway and promotes IL-2 expression. FIG. 1C. Graphs show mean+SEM of duplicates of a single PBMC donor.

### Example 3

### T Cells Expressing Both an Anti-CD79A CAR and an Anti-CD20 CCR Respond to Target Cells that Express CD20

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (SEQ ID NO: 37) or encoding an anti-CD79A CAR alone (SEQ ID NO: 18). Untransduced (UTD) cells were used as a control. After transduction, the cells were expanded in T cell growth medium containing IL-2 for 10 days. After expansion, UTD cells, anti-CD79A CAR T cells, and anti-CD79A CAR-anti-CD20 CCR T cells were co-cultured at a 1:1 ratio with CD20 negative RD cells or RD.CD20 cells (RD cells modified to express CD20). After 24 hours of co-culture, supernatants were collected and analyzed for IFNγ and IL-2 cytokines using Luminex.

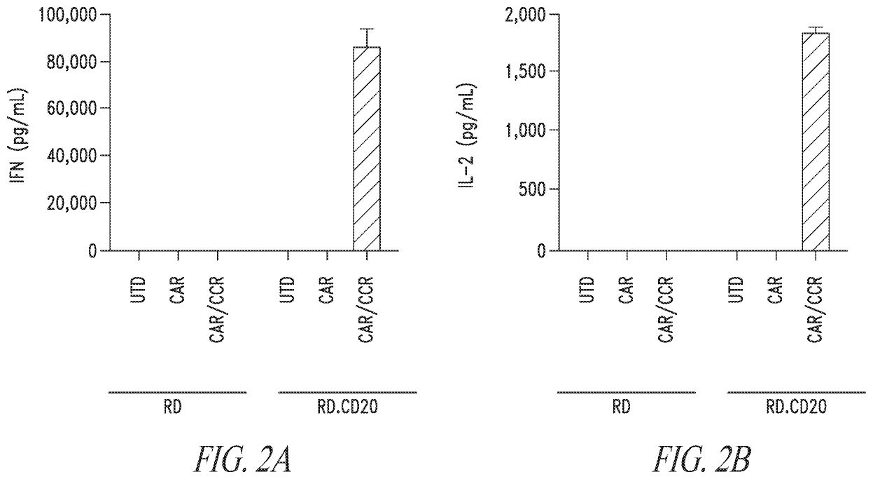

Surprisingly, anti-CD79A CAR-anti-CD20 CCR T cells produced IFNγ and IL-2 cytokine following co-culture with RD.CD20 cells. FIGS. 2A and 2B. The absence of activity against the RD parent line shows specific activity to the CD20 target. These data demonstrate CAR antigen independent activity against CCR antigen expressing cells (lacking the CAR antigen). This finding is unexpected, as CD28 signaling normally amplifies T cell receptor signaling, and therefore in the absence of CAR activity which signals through CD3ζ, it is expected that the CAR-CCR T cells will not be active against cells expressing CCR antigen alone. These data show that the anti-CD79A CAR-anti-CD20 CCR T cells may be able to engage in cytotoxic activity against both single CAR or CCR positive target cells and dual CAR and CCR positive target cells. Graphs show mean+SEM of duplicates of a single PBMC donor.

### Example 4

### T Cells Expressing Both an Anti-CD79A CAR and an Anti-CD20 CAR Show Antigen Dependent Cytokine Release

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, a T2A self-cleaving polypeptide, and an anti-CD20 CD28 CD3ζ CAR (SEQ ID NO: 41) or with a lentiviral vector comprising a polynucleotide encoding an anti-CD79A 41BB CD3ζ CAR, a T2A self-cleaving polypeptide, and an anti-CD20 41BB CD3ζ CAR (SEQ ID NO: 45). Untransduced (UTD) cells were used as a control. After transduction, the cells were expanded in T cell growth medium containing IL-2 for 10 days.

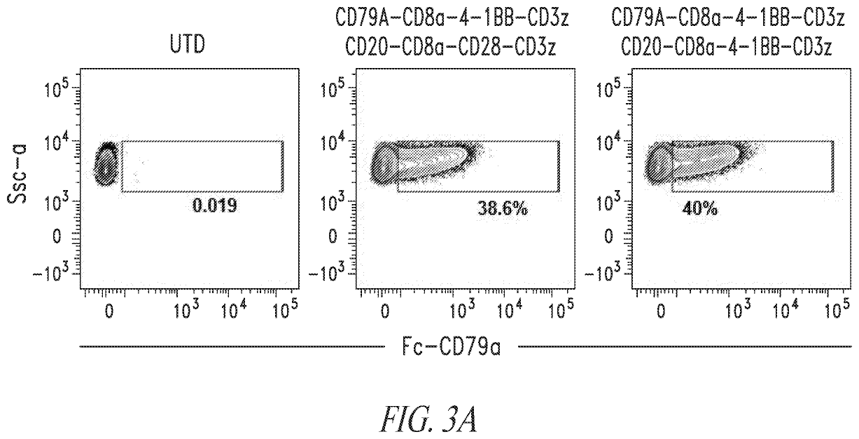

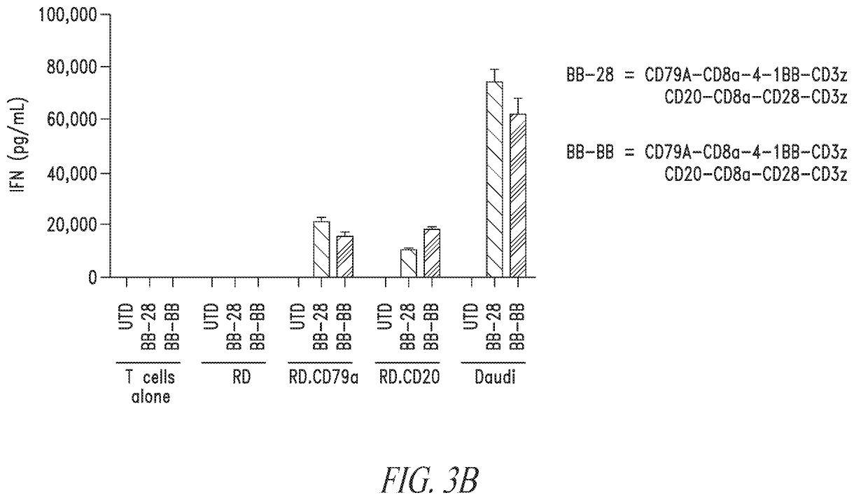

After expansion, transduced and untransduced T cell cultures were evaluated by flow cytometry for anti-CD79A CAR expression using a recombinant Fc-CD79A fusion protein conjugated to the PE fluorochrome. Expression was observed in the T cells transduced with the lentiviral vectors encoding an anti-CD79A CAR compared to untransduced control T cells. FIG. 3A.

Anti-CD79A CAR-anti-CD20 CAR T cell functionality was also evaluated. Untransduced T cells or T cells transduced with lentiviral vectors encoding an anti-CD79A 41BB CD3ζ CAR, T2A, anti-CD20 CD28 CD3ζ CAR fusion protein (SEQ ID NO: 41) or an anti-CD79A 41BB CD3ζ CAR, T2A, anti-CD20 41BB CD3ζ CAR fusion protein (SEQ ID NO: 45) were cultured alone or co-cultured at a 1:1 ratio with tumor cells lacking target antigen (RD; Rhabdosarcoma cell line); and with RD.79A, RD.CD20 cells, and Daudi cells. Following 24 hours of co-culture, supernatants were collected and analyzed for IFNγ using a Luminex assay. Both dual CD79A-CD20 CAR T cells produced IFNγ cytokine in response to RD cells expressing either target antigen alone although the responses were somewhat muted compared to the responses of CD79A CAR-CD20 CCR T cells against either antigen. Both dual CD79A-CD20 CAR T cells also showed strong responses against Daudi cells, but again, responses were somewhat muted compared to the responses of CD79A CAR-CD20 CCR T cells against Daudi cells. FIG. 3B. Graphs show mean+SEM of duplicates of a single PBMC donor.

### Example 5

T Cells Expressing Both an Anti-CD79A CAR and an Anti-CD20 CCR Show Antigen Dependent Cytokine Release

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR (SEQ ID NO: 19) and an anti-CD20 CCR (SEQ ID NO: 33). The polynucleotide is expressed as a fusion protein (SEQ ID NO: 39) wherein the anti-CD79A CAR and the anti-CD20 CCR are separated by a T2A viral self-cleaving polypeptide. Untransduced (UTD) cells were used as a control. After transduction, the cells were expanded in T cell growth medium containing IL-2 for 12 days.

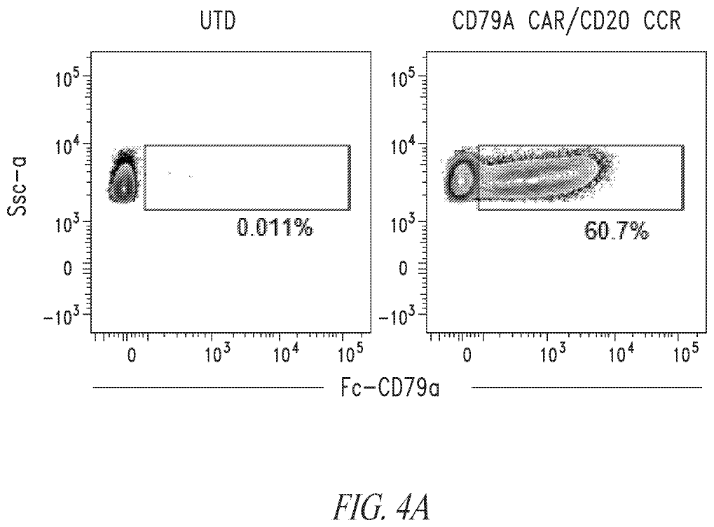

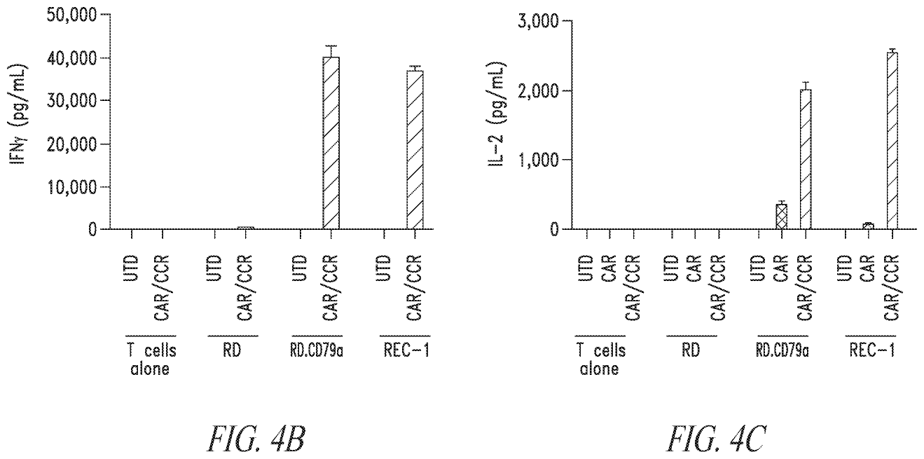

After expansion, transduced and untransduced T cell cultures were evaluated by flow cytometry for anti-CD79A CAR expression using a recombinant Fc-CD79A fusion protein conjugated to the PE fluorochrome. High expression was observed in the T cells transduced with the lentiviral vector encoding the anti-CD79A CAR compared to untransduced control T cells. FIG. 4A.

Anti-CD79A CAR-anti-CD20 CCR T cell functionality was also evaluated.

Untransduced T cells or T cells transduced with lentiviral vectors encoding an anti-CD79A CAR (SEQ ID NO: 19) or encoding an anti-CD79A 41BB CD3ζ CAR, T2A, anti-CD20 CCR fusion protein (SEQ ID NO: 39) were cultured alone or co-cultured at a 1:1 ratio with tumor cells lacking target antigen (RD; Rhabdosarcoma cell line); and with RD.79A cells (RD modified to express CD79A), RD.CD79A.CD20 cells (RD modified to express CD79A and CD20), and REC1 cells (Mantle cell lymphoma, which have high endogenous expression of both CD79a and CD20). Following 24 hours of co-culture, supernatants were collected and analyzed for IFNγ and IL-2 cytokines using a Luminex assay. Anti-CD79A CAR-anti-CD20 CCR T cells produced IFNγ cytokine in response to only the cell lines expressing CD79A, but not against RD cells. FIG. 4B. IL-2 production was increased in anti-CD79A CAR-anti-CD20 CCR T cells compared to anti-CD79A CAR T cells because the activity of the anti-CD20 CCR through its CD28 costimulatory domain induces the PI3K pathway and promotes IL-2 expression. FIG. 4C. Graphs show mean+SEM of duplicates of a single PBMC donor.

### Example 6

### T Cells Expressing Both an Anti-CD79A CAR and an Anti-CD20 CCR Respond to Target Cells that Express CD20

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (SEQ ID NO: 39) or encoding an anti-CD79A CAR alone (SEQ ID NO: 19). Untransduced (UTD) cells were used as a control. After transduction, the cells were expanded in T cell growth medium containing IL-2 for 10 days. After expansion, UTD cells, anti-CD79A CAR T cells, and anti-CD79A CAR-anti-CD20 CCR T cells were co-cultured at a 1:1 ratio with CD20 negative RD cells or RD.CD20 cells (RD cells modified to express CD20). After 24 hours of co-culture, supernatants were collected and analyzed for IFNγ and IL-2 cytokines using Luminex.

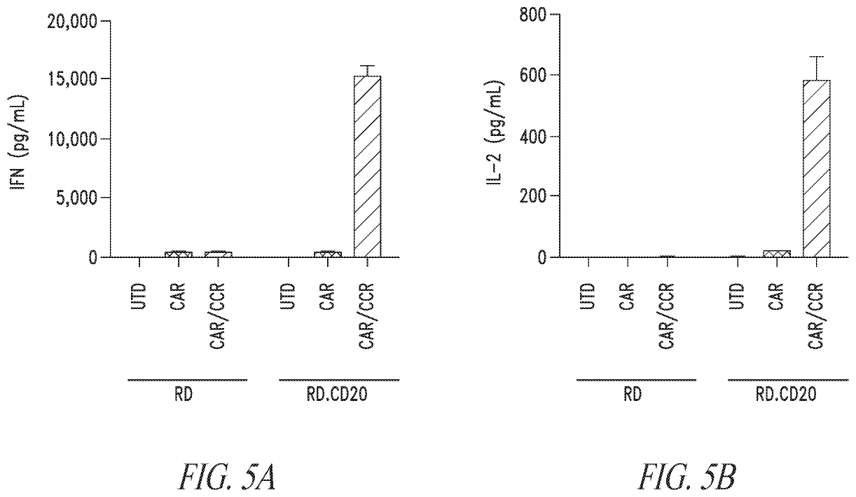

Surprisingly, anti-CD79A CAR-anti-CD20 CCR T cells produced IFNγ and IL-2 cytokine following co-culture with RD.CD20 cells. FIGS. 5A and 5B. The absence of activity against the RD parent line shows specific activity to the CD20 target. These data demonstrate CAR antigen independent activity against CCR antigen expressing cells (lacking the CAR antigen). This finding is unexpected, as CD28 signaling normally amplifies T cell receptor signaling, and therefore in the absence of CAR activity which signals through CD3ζ, it is expected that the CAR-CCR T cells will not be active against cells expressing CCR antigen alone. These data show that the anti-CD79A CAR-anti-CD20 CCR T cells may be able to engage in cytotoxic activity against both single CAR or CCR positive target cells and dual CAR and CCR positive target cells. Graphs show mean+SEM of duplicates of a single PBMC donor.

### Example 7

### T Cells Expressing Both an Anti-CD79A CAR and an Anti-CD20 CAR Show Antigen Dependent Cytokine Release

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, a T2A self-cleaving polypeptide, and an anti-CD20 CD28 CD3ζ CAR (SEQ ID NO: 43) or with a lentiviral vector comprising a polynucleotide encoding an anti-CD79A 41BB CD3ζ CAR, a T2A self-cleaving polypeptide, and an anti-CD20 41BB CD3ζ CAR (SEQ ID NO: 47). Untransduced (UTD) cells were used as a control. After transduction, the cells were expanded in T cell growth medium containing IL-2 for 10 days.

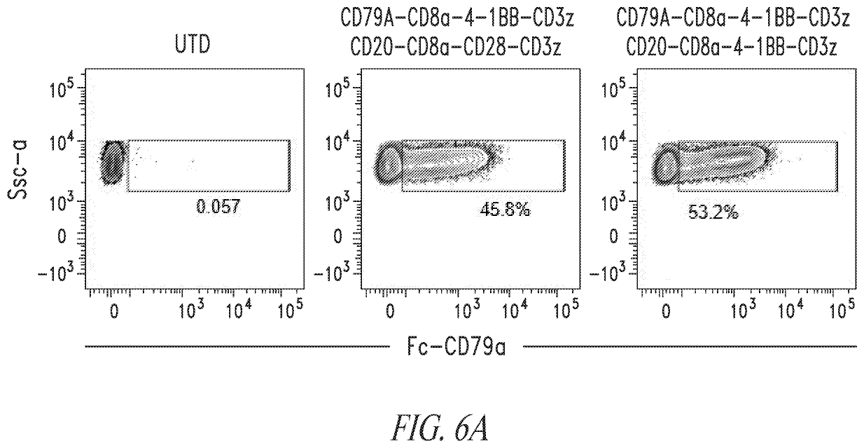

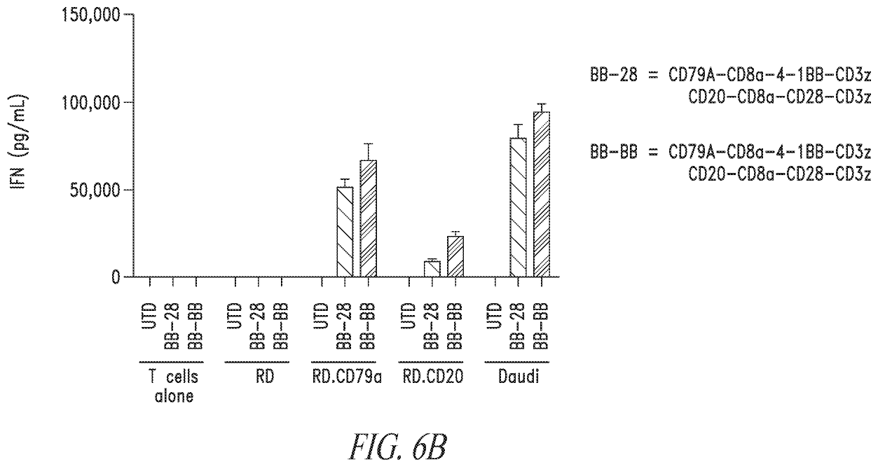

After expansion, transduced and untransduced T cell cultures were evaluated by flow cytometry for anti-CD79A CAR expression using a recombinant Fc-CD79A fusion protein conjugated to the PE fluorochrome. Expression was observed in the T cells transduced with the lentiviral vectors encoding an anti-CD79A CAR compared to untransduced control T cells. FIG. 6A.

Anti-CD79A CAR-anti-CD20 CAR T cell functionality was also evaluated. Untransduced T cells or T cells transduced with lentiviral vectors encoding an anti-CD79A 41BB CD3ζ CAR, T2A, anti-CD20 CD28 CD3ζ CAR fusion protein (SEQ ID NO: 43) or an anti-CD79A 41BB CD3ζ CAR, T2A, anti-CD20 41BB CD3ζ CAR fusion protein (SEQ ID NO: 47) were cultured alone or co-cultured at a 1:1 ratio with tumor cells lacking target antigen (RD; Rhabdosarcoma cell line); and with RD.79A, RD.CD20 cells, and Daudi cells. Following 24 hours of co-culture, supernatants were collected and analyzed for IFNγ using a Luminex assay. Both dual CD79A-CD20 CAR T cells produced IFNγ cytokine in response to RD cells expressing either target antigen alone although the responses were somewhat muted compared to the responses of CD79A CAR-CD20 CCR T cells against either antigen. Both dual CD79A-CD20 CAR T cells also showed strong responses against Daudi cells, but again, responses were somewhat muted compared to the responses of CD79A CAR-CD20 CCR T cells against Daudi cells. FIG. 6B. Graphs show mean+SEM of duplicates of a single PBMC donor.

### Example 8

### T Cells Expressing an Anti-CD79A CAR and Anti-CD20 CCR Effectively Kill Cell Lines Expressing CD79A or CD20

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (e.g., SEQ ID NO: 37 or 39). After transduction, the cells were expanded in T cell growth medium containing IL-2 for 10 days.

After expansion, transduced and untransduced T cell cultures were evaluated for in vitro cytotoxic function using non-invasive electrical impedance monitoring by the xCELLigence Real Time Cell Analysis (RTCA).

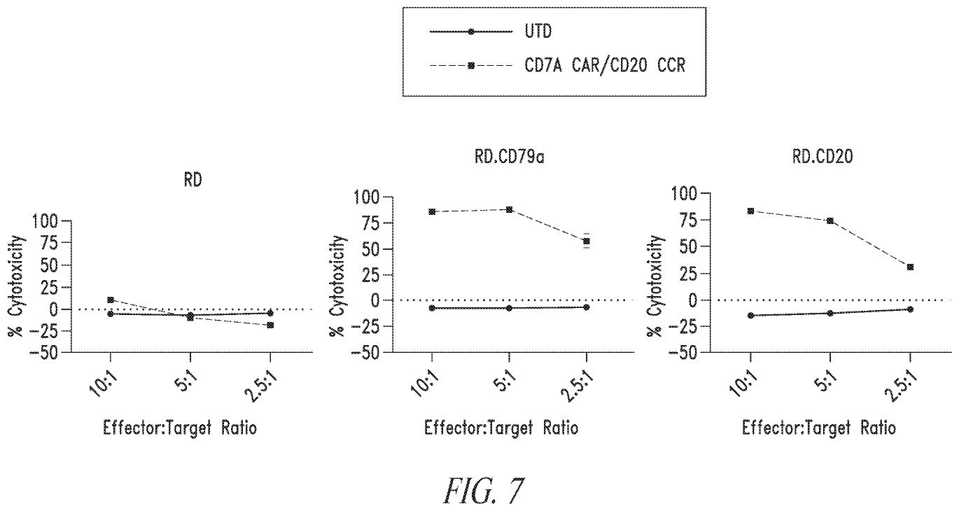

UTD T cells and T cells expressing the anti-CD79A CAR and anti-CD20 CCR were co-cultured at E:T ratios of 10:1, 5:1, and 2.5:1 ratios with RD (Rhabdosarcoma) cell lines or RD cell lines engineered to express either CD79A (RD.CD79A) or CD20 (RD.CD20). After 6 hours of co-culture, cell index was measured by noninvasive electrical impedance on the CELLigence RTCA MP. Percent cytotoxicity was calculated by normalizing the T cell condition cell index to tumor alone control cell index. The absence of activity against RD parent cells showed specific activity to the CD79A CAR target and the CD20 CCR target. FIG. 7 (left panel). Transduced T cells exhibited cytotoxicity to both RD.CD79A cells (FIG. 7, center panel) and RD.CD20 cell (FIG. 7, right panel). Panels show mean+SD cytotoxicity at 10:1, 5:1, 2.5:1 Effector:Target ratios of duplicates of a single PBMC donor.

The ability of T cells expressing the anti-CD79A CAR and anti-CD20 CCR to kill both CD79a and CD20 single positive targets demonstrates the novel dual targeting ability of these cells. Without wishing to be bound by any particular theory, it is believed that CCR mediated cytotoxicity can occur through CD20 engagement independent of a CAR target and is a unique and innovative trait of these cells.

### Example 9

### T Cells Expressing an Anti-CD79A CAR and Anti-CD20 CCR are Efficacious in a Daudi (Burkitt Lymphoma) Tumor Model

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (SEQ ID NO: 37). After transduction, the cells were expanded in T cell growth medium containing IL-2 for 10 days, then cryopreserved or evaluated for function.

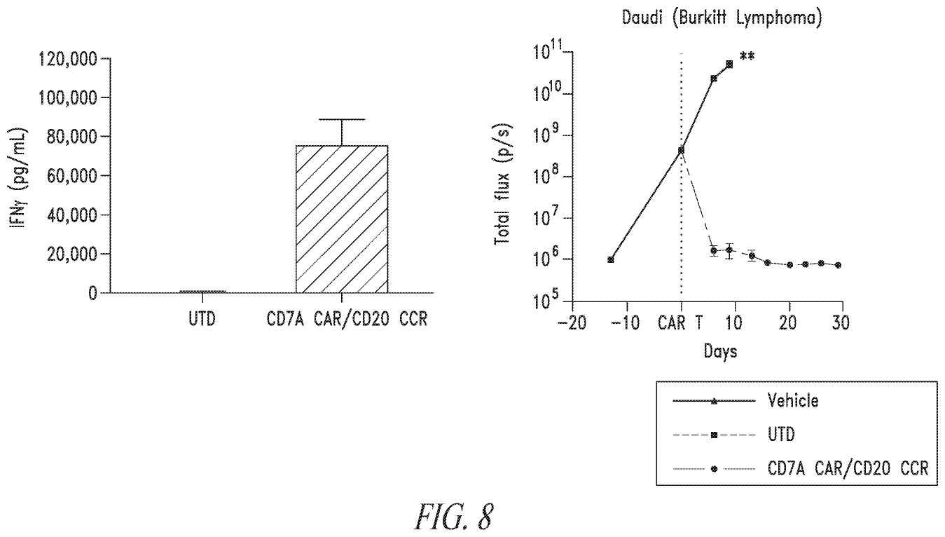

In Vitro. UTD T cells or T cells expressing an anti-CD79A 41BB CD3ζ CAR, T2A, anti-CD20 CCR fusion protein were co-cultured at a 1:1 ratio with Daudi tumor cells (CD79A⁺, CD20⁺) for 24 hours and then supernatants were collected and analyzed for IFNγ using Luminex. FIG. 8 (left panel) shows mean+SEM of IFNγ cytokine of duplicate assays across 3 PBMC donors.

In Vivo. NSG mice were injected with 2×10⁶ luciferase-expressing Daudi tumor cells. After 13 days, mice (5) were injected with Vehicle (medium), 10×10⁶ UTD T cells, or 10×10⁶ T cells expressing the anti-CD79A CAR and anti-CD20 CCR. After 20 days, only the CD79A CAR/CD20 CCR treated mice cleared the tumor cells and maintained clearance to the end of study day 30. FIG. 8 (right panel) shows mean+SEM across N=5 mice for Vehicle except time points after day 6 where N=4, N=5 for UTD and N=5 for CD79A CAR/CD20 CCR. *Asterisks indicate animal sacrifice due to tumor size and animal health.

### Example 10

### T Cells Expressing an Anti-CD79A CAR and Anti-CD20 CCR are Efficacious in a NU-DUL-1 ABC DLBCL Tumor Model

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (SEQ ID NO: 37). After transduction, the cells were expanded in T cell growth medium containing IL-2 for 10 days, then cryopreserved or evaluated for function.

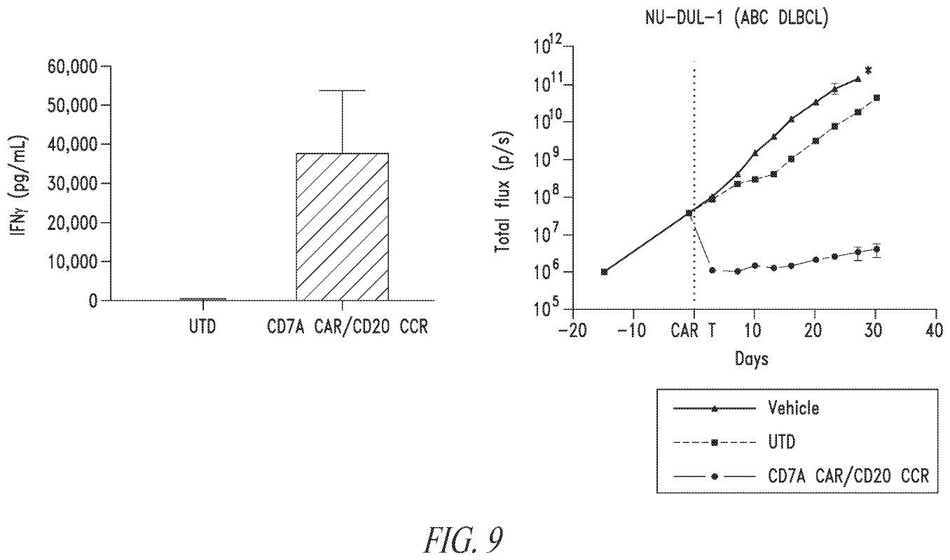

In Vitro. UTD T cells or T cells expressing an anti-CD79A 41BB CD3ζ CAR, T2A, anti-CD20 CCR fusion protein were co-cultured at a 1:1 ratio with NU-DUL-1 ABC cells (CD79A⁺, CD20⁺) for 24 hours and then supematants were collected and analyzed for IFNγ using Luminex. FIG. 9 (left panel) shows mean+SEM of IFNγ cytokine of duplicate assays across 3 PBMC donors.

In Vivo. NSG mice were injected with 10×10⁶ luciferase-expressing NU-DUL-1 ABC tumor cells. After 15 days, mice (5) were injected with Vehicle (medium), 10×10⁶ UTD T cells, or 5×10⁶ T cells expressing the anti-CD79A CAR and anti-CD20 CCR. Only the CD79A CAR/CD20 CCR treated mice caused tumor regression in this model. FIG. 9 (right panel) shows mean+SEM across N=5 mice for Vehicle except time points after day 23 where N=4, N=5 for UTD and N=5 for CD79A CAR/CD20 CCR. *Asterisks indicate animal sacrifice due to tumor size and animal health.

### Example 11

### T Cells Expressing an Anti-CD79A CAR and Anti-CD20 CCR are Efficacious in a Toledo Germinal Center B Cell (GCB) DLBCL Tumor Model

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (SEQ ID NO: 37). After transduction, the cells were expanded in T cell growth medium containing IL-2 for 10 days, then cryopreserved or evaluated for function.

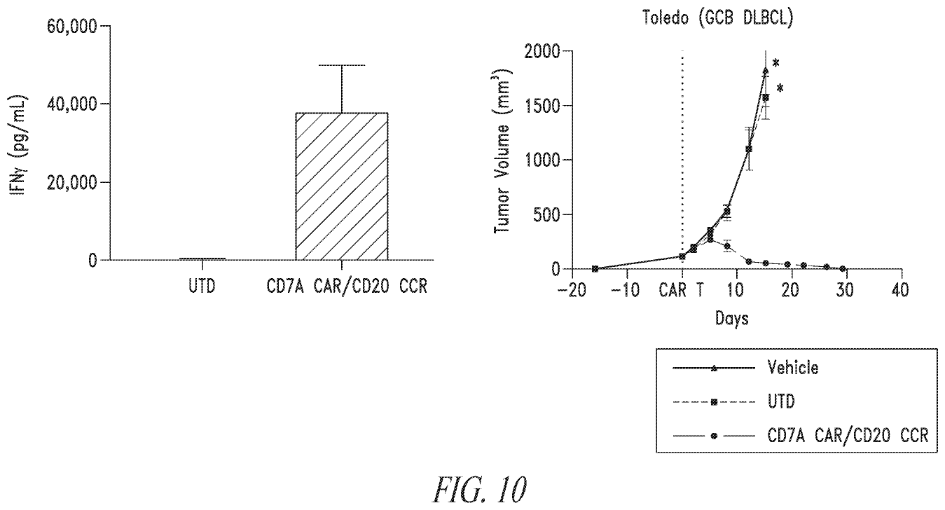

In Vitro. UTD T cells or T cells expressing an anti-CD79A 41BB CD3ζ CAR, T2A, anti-CD20 CCR fusion protein were co-cultured at a 1:1 ratio with Toledo GCB DLBCL cells (CD79A^{low}, CD20^{high}) for 24 hours and then supernatants were collected and analyzed for IFNγ using Luminex. FIG. 10 (left panel) shows mean+SEM of IFNγ cytokine of duplicate assays across 3 PBMC donors.

In Vivo. NSG mice were injected with 50×10⁶ luciferase-expressing Toledo GCB DLBCL tumor cells. After 16 days (tumors ˜100 mm³), mice (5) were injected with Vehicle (medium), 20×10⁶ UTD T cells, or 2.5×10⁶ T cells expressing the anti-CD79A CAR and anti-CD20 CCR. Only the CD79A CAR/CD20 CCR treated mice cleared the tumor cells and maintained clearance to the end of study day 29, tumor was completely cleared in 3/5 mice. FIG. 10 (right panel) shows mean+SEM across N=5 mice for each condition. *Asterisks indicate animal sacrifice due to tumor size and animal health.

### Example 12

### CBLB Edited T Cells Expressing an Anti-CD79A CAR and Anti-CD20 CCR Show Increased Efficacy in a Toledo GCB DLBCL Tumor Model

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (SEQ ID NO: 37). Three days after activation, the transduced cells were electroporated with mRNA encoding a megaTAL that cleaves the CBLB gene (SEQ ID NO: 54), and expanded in T cell growth medium containing IL-2 for 10 days, then cryopreserved or evaluated for function.

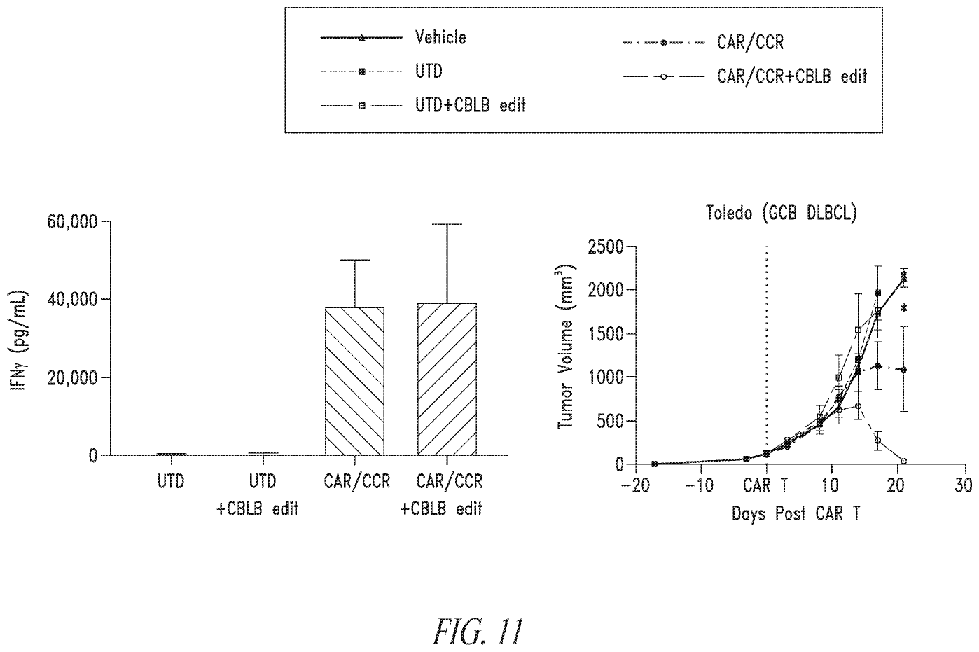

In Vitro. UTD T cells (+/−CBLB edit) or T cells expressing an anti-CD79A 41BB CD3ζ CAR, T2A, anti-CD20 CCR fusion protein (+/−CBLB edit) were co-cultured at a 1:1 ratio with Toledo GCB DLBCL cells (CD79A^{low}, CD20^{high}) for 24 hours and then supernatants were collected and analyzed for IFNγ using Luminex. FIG. 11 (left panel) shows mean+SEM of IFNγ cytokine of duplicate assays across 3 PBMC donors.

In Vivo. NSG mice were injected with 50×10⁶ luciferase-expressing Toledo GCB DLBCL tumor cells. After 17 days (tumors ˜130 mm³), mice (5) were injected with Vehicle (medium), 5×10⁶ UTD T cells (+/−CBLB edit), or 1×10⁶ T cells expressing the anti-CD79A CAR and anti-CD20 CCR (+/−CBLB edit). Mice treated with CBLB edited T cells expressing the CD79A CAR/CD20 CCR treated mice cleared the tumor cells and maintained clearance to the end of study day 21, tumor was completely cleared in 1/5 mice. FIG. 11 (right panel) shows N=5 mice for Vehicle except time points after day 17 where N=3, N=5 for CD79A CAR/CD20 CCR except time points after day 21 where N=4, and N=5 for CD79A CAR/CD20 CCR (+CBLB edit). *Asterisks indicate animal sacrifice due to tumor size and animal health.

### Example 13

### CBLB Edited T Cells Expressing an Anti-CD79A CAR and Anti-CD20 CCR Show Increased Efficacy in a Daudi CD20 Knockout Tumor Model

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (SEQ ID NO: 37). Three days after activation, the transduced cells were electroporated with mRNA encoding a megaTAL that cleaves the CBLB gene (SEQ ID NO: 54) and expanded in T cell growth medium containing IL-2 for 10 days, then cryopreserved or evaluated for function.

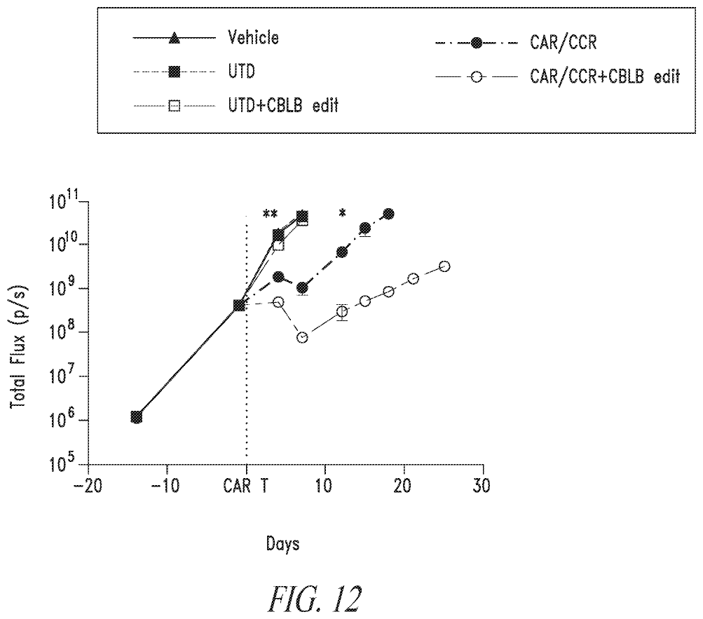

In Vivo. NSG mice were injected with 2×10⁶ luciferase-expressing Daudi.CD20KO cells (CD79A⁺, CD20⁻). After 14 days, mice (5) were injected with Vehicle (medium), 20×10⁶ UTD T cells (+/−CBLB edit), or 10×10⁶ T cells expressing the anti-CD79A CAR and anti-CD20 CCR (+/−CBLB edit). Mice treated with T cells expressing the CD79A CAR/CD20 CCR showed anti-tumor activity. CBLB edited CD79A CAR/CD20 CCR expressing T cells showed enhanced anti-tumor activity compared to all other conditions. FIG. 12 shows mean+SEM across N=5 mice for all conditions. *Asterisks indicate animal sacrifice due to tumor size and animal health.

### Example 14

### Cytokine Secretion in CBLB Edited T Cells

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (SEQ ID NO: 37). Three days after activation, the transduced cells were electroporated with mRNA encoding a megaTAL that cleaves the CBLB gene (SEQ ID NO: 54) and expanded in T cell growth medium containing IL-2 for 10 days, then cryopreserved or evaluated for function.

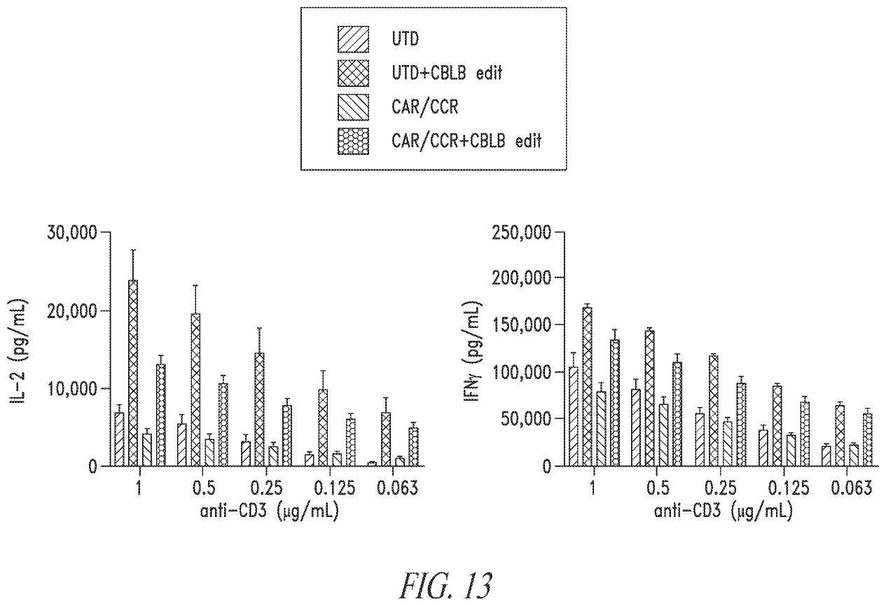

UTD T cells (+/−CBLB edit), or T cells expressing the anti-CD79A CAR and anti-CD20 CCR (+/−CBLB edit) plated at a concentration of 1×10⁶ cells/mL with T cell growth medium lacking exogenous IL-2 in 96-well high binding plates previously coated with CD3 (1 μg/mL to 0.063 μg/mL) and CD28 (5 μg/mL) monoclonal antibodies. After 24 hours supernatants were harvested and cytokine detection was measured via Luminex. CBLB edited UTD T cells and CD79A CAR/CD20 CCR expressing cells showed increased IL-2 (FIG. 13, left panel) and IFN γ (FIG. 13, right panel) production. UTD T cell conditions were plated in duplicate and transduced T cell conditions were plated in quadruplicate. FIG. 13 shows mean+SEM across N=3 donors.

### Example 15

CBLB Edited T Cells Expressing an Anti-CD79A CAR and Anti-CD20 CCR Show Increased IL-2 Secretion in a Daudi Tumor Model

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (SEQ ID NO: 37). Three days after activation, the transduced cells were electroporated with mRNA encoding a megaTAL that cleaves the CBLB gene (SEQ ID NO: 54) and expanded in T cell growth medium containing IL-2 for 10 days, then cryopreserved or evaluated for function.

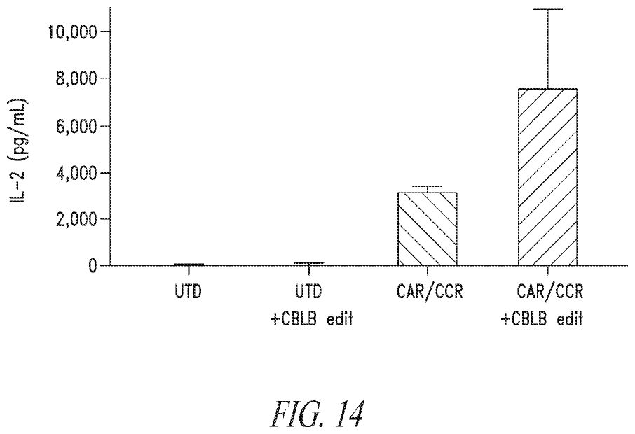

UTD T cells (+/−CBLB edit) or T cells expressing an anti-CD79A 41BB CD3ζ CAR, T2A, anti-CD20 CCR fusion protein (+/−CBLB edit) were co-cultured at a 1:1 ratio with Daudi cells for 24 hours and then supernatants were collected and analyzed for IL-2 using Luminex. CBLB edited T cells expressing CD79A CAR/CD20 CCR produced increased amounts of IL-2 compared to all other conditions tested. FIG. 14 shows mean+SEM of IL-2 cytokine production of duplicates across a single PBMC donor.

### Example 16

### Proliferation of CBLB Edited T Cells

PBMCs were harvested from healthy human donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (SEQ ID NO: 37). Three days after activation, the transduced cells were electroporated with mRNA encoding a megaTAL that cleaves the CBLB gene (SEQ ID NO: 54) or mRNA that encodes an inactive TCRα megaTAL (TCRα^{DEAD}) and expanded in T cell growth medium containing IL-2 for 10 days, then cryopreserved or evaluated for function.

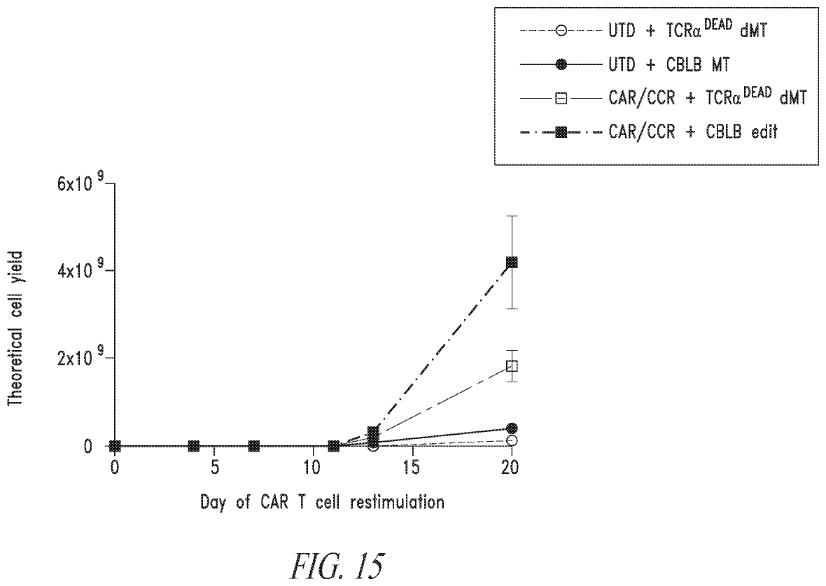

GFP-expressing Daudi cells were plated at a density of 50,000 cells per well in 96-well plates in T cell growth medium containing low level IL-2. The Daudi cells were co-cultured with 50,000 UTD T cells (CBLB or TCRα^{DEAD}) or T cells expressing an anti-CD79A 41BB CD3ζ CAR, T2A, anti-CD20 CCR fusion protein (CBLB or TCRα^{DEAD}). After four days, half the T cell growth medium was removed and replaced with fresh medium containing low level IL-2. Three days later, T cells were resuspended and counted. A total of 50,000 T cells (from each condition) were then transferred to a fresh 96-well plate containing 50,000 Daudi-GFP tumor cells. Four days later (day 11) medium was removed and replaced with fresh medium containing low level IL-2. Three days later (day 14), T cells were resuspended and counted. A total of 50,000 T cells (UTD or transduced (+/−edit)) were then transferred to a fresh 96-well plate containing 50,000 Daudi-GFP tumor cells. Three days later (day 17) medium was removed and replaced with fresh medium containing low level IL-2. Three days later (day 20), T cells were resuspended and counted. After the third round of tumor cell stimulation, CBLB edited T cells expressing a CD79 CAR/CD20 CCR show increased proliferation compared to TCRα dead control treated cells. FIG. 15 shows mean+SD of 4 replicates per condition.

### Example 17

### CBLB Edited T Cells Expressing an Anti-CD79A CAR and Anti-CD20 CCR Show Increased Ifnγ in a Toledo GCB DLBCL Tumor Model

PBMCs were harvested from three healthy human donors and three DLBCL-diseased donors and activated using anti-CD3 and anti-CD28 antibodies. Activated cells were transduced with a lentiviral vector comprising a polynucleotide encoding an anti-CD79A 41BB CD3ζ CAR, T2A, anti-CD20 CCR fusion protein (SEQ ID NO: 37). Three days after activation, the transduced cells were electroporated with mRNA encoding a megaTAL that cleaves the CBLB gene (SEQ ID NO: 54) and expanded in T cell growth medium containing IL-2 for 10 days, then cryopreserved or evaluated for function.

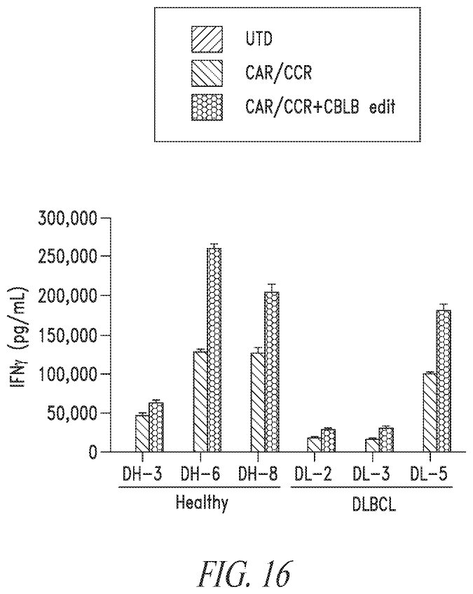

UTD T cells or T cells expressing an anti-CD79A 41BB CD3ζ CAR, T2A, anti-CD20 CCR fusion protein (+/−CBLB edit) were co-cultured at a 1:1 ratio with Toledo GCB DLBCL cells (CD79A^{low}, CD20^{high}) for 24 hours and then supernatants were collected and analyzed for IFNγ using Luminex. CBLB-edited T cells expressing an anti-CD79A 41BB CD3ζ CAR, T2A, anti-CD20 CCR fusion protein produced more IFNγ cytokine in response to tumor cells compared to UTD or non-edited CD79A CAR/CD20 CCR expressing T cells in all healthy and DLBCL donors. FIG. 16 (left panel) shows mean+SEM of IFNγ cytokine of two replicates assays across all donors.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A fusion polypeptide comprising an anti-CD79A chimeric antigen receptor (CAR), a polypeptide cleavage signal, and an anti-CD20 chimeric costimulatory receptor (CCR).

2. The fusion polypeptide of claim 1, wherein the anti-CD79A CAR comprises an anti-CD79A antibody or antigen binding fragment thereof; a first transmembrane domain; a first intracellular costimulatory signaling domain; and a primary signaling domain.

3. The fusion polypeptide of claim 1 or claim 2, wherein the anti-CD79A CAR comprises an anti-CD79A antibody or antigen binding fragment thereof selected from the group consisting of: a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, Nanobody).

4. The fusion polypeptide of any one of claims 1 to 3, wherein the anti-CD79A CAR comprises an anti-CD79A antibody or antigen binding fragment thereof that is an scFv.

5. The fusion polypeptide of any one of claims 1 to 4, wherein the anti-CD79A CAR comprises an anti-CD79A antibody or antigen binding fragment thereof comprising a variable light chain sequence comprising CDRL1-CDRL3 sequences set forth in SEQ ID NOs: 1-3 or 9-11 and a variable heavy chain sequence comprising CDRH1-CDRH3 sequences set forth in SEQ ID NOs: 4-6 or 12-14.

6. The fusion polypeptide of any one of claims 1 to 5, wherein the anti-CD79A CAR comprises an anti-CD79A antibody or antigen binding fragment thereof comprising the light chain CDRs as set forth in SEQ ID NOs: 1-3 and the heavy chain CDRs as set forth in SEQ ID NOs: 4-6.

7. The fusion polypeptide of any one of claims 1 to 5, wherein the anti-CD79A CAR comprises an anti-CD79A antibody or antigen binding fragment thereof comprising the light chain CDRs as set forth in SEQ ID NOs: 9-11 and the heavy chain CDRs as set forth in SEQ ID NOs: 12-14.

8. The fusion polypeptide of any one of claims 1 to 5, wherein the anti-CD79A CAR comprises an anti-CD79A antibody or antigen binding fragment thereof comprising a variable light chain sequence as set forth in any one of SEQ ID NOs: 7 or 15 and a variable heavy chain sequence as set forth in any one of SEQ ID NOs: 8 or 16.

9. The fusion polypeptide of any one of claims 1 to 5, wherein the anti-CD79A CAR comprises an anti-CD79A antibody or antigen binding fragment thereof comprising a variable light chain sequence as set forth in SEQ ID NO: 7 and/or a variable heavy chain sequence as set forth in SEQ ID NO: 8.

10. The fusion polypeptide of any one of claims 1 to 5, wherein the anti-CD79A CAR comprises an anti-CD79A antibody or antigen binding fragment thereof comprising a variable light chain sequence as set forth in SEQ ID NO: 15 and/or a variable heavy chain sequence as set forth in SEQ ID NO: 16.

11. The fusion polypeptide of any one of claims 1 to 10, wherein the anti-CD79A CAR comprises a first transmembrane domain isolated from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1.

12. The fusion polypeptide of any one of claims 1 to 11, wherein the anti-CD79A CAR comprises a first transmembrane domain isolated from CD8α.

13. The fusion polypeptide of any one of claims 1 to 12, wherein the anti-CD79A CAR comprises a first costimulatory signaling domain isolated from a costimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

14. The fusion polypeptide of any one of claims 1 to 13, wherein the anti-CD79A CAR comprises a first costimulatory signaling domain isolated from CD137.

15. The fusion polypeptide of any one of claims 1 to 14, wherein the anti-CD79A CAR comprises a primary signaling domain isolated from a polypeptide selected from the group consisting of: FcRγ, FcRβ, CD3γ, CD3δ, CD3ε, CD3ζ, CD22, CD79a, CD79b, and CD66d.

16. The fusion polypeptide of any one of claims 1 to 15, wherein the anti-CD79A CAR comprises a primary signaling domain isolated from CD3ζ.

17. A fusion polypeptide comprising an anti-CD79A CAR comprising a variable light chain sequence as set forth in any one of SEQ ID NOs: 7 or 15 and a variable heavy chain sequence as set forth in any one of SEQ ID NOs: 8 or 16, a CD8a hinge domain, a CD8α transmembrane domain, a CD137 costimulatory domain and a CD3ζ primary signaling domain, a polypeptide cleavage signal, and an anti-CD20 CCR.

18. The fusion polypeptide of any one of claims 1 to 17, wherein the polypeptide cleavage signal is a viral self-cleaving polypeptide.

19. The fusion polypeptide of any one of claims 1 to 18, wherein the polypeptide cleavage signal is a viral self-cleaving 2A polypeptide.

20. The fusion polypeptide of any one of claims 1 to 19, wherein the polypeptide cleavage signal is a viral self-cleaving polypeptide selected from the group consisting of: a foot-and-mouth disease virus (FMDV) 2A (F2A) peptide, an equine rhinitis A virus (ERAV) 2A (E2A) peptide, a Thosea asigna virus (TaV) 2A (T2A) peptide, a porcine teschovirus-1 (PTV-1) 2A (P2A) peptide, a Theilovirus 2A peptide, and an encephalomyocarditis virus 2A peptide.

21. A fusion polypeptide comprising an anti-CD79A CAR comprising an amino acid sequence as set forth in any one of SEQ ID NOs: 17-20, a T2A self-cleaving polypeptide, and an anti-CD20 CCR.

22. The fusion polypeptide of any one of claims 1 to 21, wherein the anti-CD20 CCR comprises an anti-CD20 antibody or antigen binding fragment thereof, a second transmembrane domain, and a second intracellular costimulatory domain.

23. The fusion polypeptide of any one of claims 1 to 22, wherein the anti-CD20 CCR comprises an anti-CD20 antibody or antigen binding fragment thereof selected from the group consisting of a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, Nanobody).

24. The fusion polypeptide of any one of claims 1 to 23, wherein the anti-CD20 CCR comprises an anti-CD20 antibody or antigen binding fragment thereof that is an scFv.

25. The fusion polypeptide of any one of claims 1 to 24, wherein the anti-CD20 CCR comprises an anti-CD20 antibody or antigen binding fragment thereof comprising a variable light chain sequence comprising CDRL1-CDRL3 sequences set forth in SEQ ID NOs: 25-27 and a variable heavy chain sequence comprising CDRH1-CDRH3 sequences set forth in SEQ ID NOs: 28-30.

26. The fusion polypeptide of any one of claims 1 to 25, wherein the anti-CD20 CCR comprises an anti-CD20 antibody or antigen binding fragment thereof comprising a variable light chain sequence as set forth in SEQ ID NO: 31 and a variable heavy chain sequence as set forth in SEQ ID NO: 32.

27. The fusion polypeptide of any one of claims 1 to 26, wherein the anti-CD20 CCR comprises a second transmembrane domain isolated from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1.

28. The fusion polypeptide of any one of claims 1 to 27, wherein the anti-CD20 CCR comprises a second transmembrane domain isolated from CD8α.

29. The fusion polypeptide of any one of claims 1 to 28, wherein the anti-CD20 CCR comprises a second costimulatory signaling domain isolated from a costimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

30. The fusion polypeptide of any one of claims 1 to 29, wherein the anti-CD20 CCR comprises a second costimulatory signaling domain is isolated from CD28.

31. The fusion polypeptide of any one of claims 1 to 30, wherein the anti-CD20 CCR comprises a CD8α hinge domain, a CD8α transmembrane domain, and a CD28 costimulatory signaling domain.

32. A fusion polypeptide comprising an anti-CD79A CAR comprising the amino acid sequence set forth in any one of SEQ ID NOs: 17-20, a T2A self-cleaving polypeptide, and an anti-CD20 CCR comprising the amino acid sequence set forth in SEQ ID NO: 33 or SEQ ID NO: 35.

33. A fusion polypeptide comprising the amino acid sequence set forth in SEQ ID NO: 37 or SEQ ID NO: 39.

34. A polynucleotide encoding the anti-CD79A CAR and the anti-CD20 CCR of any one of claims 1 to 33.

35. A polynucleotide encoding the fusion polypeptide of any one of claims 1 to 33.

36. A polynucleotide comprising a sequence set forth in SEQ ID NO: 38 or SEQ ID NO: 40.

37. A vector comprising the polynucleotide encoding the fusion polypeptide of any one of claims 1 to 33 or the polynucleotide of any one of claims 34 to 36.

38. The vector of claim 37, wherein the vector is an expression vector.

39. The vector of claim 37 or claim 38, wherein the vector is an episomal vector.

40. The vector of any one of claims 37 to 39, wherein the vector is a viral vector.

41. The vector of any one of claims 37 to 40, wherein the vector is a retroviral vector.

42. The vector of any one of claims 37 to 41, wherein the vector is a lentiviral vector.

43. A cell that expresses the fusion polypeptide of any one of claims 1 to 33.

44. A cell comprising a polynucleotide encoding the fusion polypeptide of any one of claims 1 to 33, the polynucleotide of any one of claims 34 to 36, or the vector of any one of claims 36 to 42.

45. A cell comprising one or more polynucleotides encoding:
(a) an anti-CD79A CAR comprising an anti-CD79A antibody or antigen binding fragment thereof, a first transmembrane domain; a first intracellular costimulatory signaling domain; and a primary signaling domain; and
(b) an anti-CD20 CCR comprising an anti-CD20 antibody or antigen binding fragment thereof, a second transmembrane domain; a second intracellular costimulatory signaling domain.

46. The cell of claim 45, wherein the anti-CD79A antibody or antigen binding fragment thereof and the anti-CD20 antibody or antigen binding fragment thereof are both independently selected from the group consisting of: a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, Nanobody).

47. The cell of claim 45 or claim 46, wherein the anti-CD79A antibody or antigen binding fragment thereof and the anti-CD20 antibody or antigen binding fragment thereof are both scFvs.

48. The cell of any one of claims 45 to 47, wherein the anti-CD79A antibody or antigen binding fragment thereof comprises a variable light chain sequence comprising CDRL1-CDRL3 sequences set forth in SEQ ID NOs: 1-3 or 9-11 and a variable heavy chain sequence comprising CDRH1-CDRH3 sequences set forth in SEQ ID NOs: 4-6 or 12-14.

49. The cell of any one of claims 45 to 48, wherein the anti-CD79A antibody or antigen binding fragment thereof comprises the light chain CDRs as set forth in SEQ ID NOs: 1-3 and the heavy chain CDRs as set forth in SEQ ID NOs: 4-6.

50. The cell of any one of claims 45 to 48, wherein the anti-CD79A antibody or antigen binding fragment thereof comprises the light chain CDRs as set forth in SEQ ID NOs: 9-11 and the heavy chain CDRs as set forth in SEQ ID NOs: 12-14.

51. The cell of any one of claims 45 to 48, wherein the anti-CD79A antibody or antigen binding fragment thereof comprises a variable light chain sequence as set forth in any one of SEQ ID NOs: 7 or 15 and a variable heavy chain sequence as set forth in any one of SEQ ID NOs: 8 or 16.

52. The cell of any one of claims 45 to 48, wherein the anti-CD79A antibody or antigen binding fragment thereof comprises a variable light chain sequence as set forth in SEQ ID NO: 7 and/or a variable heavy chain sequence as set forth in SEQ ID NO: 8.

53. The cell of any one of claims 45 to 48, wherein the anti-CD79A antibody or antigen binding fragment thereof comprises a variable light chain sequence as set forth in SEQ ID NO: 15 and/or a variable heavy chain sequence as set forth in SEQ ID NO: 16.

54. The cell of any one of claims 45 to 53, wherein the anti-CD20 antibody or antigen binding fragment thereof comprises a variable light chain sequence comprising CDRL1-CDRL3 sequences set forth in SEQ ID NOs: 25-27 and a variable heavy chain sequence comprising CDRH1-CDRH3 sequences set forth in SEQ ID NOs: 28-30.

55. The cell of any one of claims 45 to 53, wherein the anti-CD20 antibody or antigen binding fragment thereof comprising a variable light chain sequence as set forth in SEQ ID NO: 31 and a variable heavy chain sequence as set forth in SEQ ID NO: 32.

56. The cell of any one of claims 45 to 55, wherein the first transmembrane domain and the second transmembrane domain are each independently isolated from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1.

57. The cell of any one of claims 45 to 56, wherein the first transmembrane domain and the second transmembrane domain are both isolated from CD8α.

58. The cell of any one of claims 45 to 57, wherein the first costimulatory signaling domain and the second costimulatory domain are each independently isolated from a costimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

59. The cell of any one of claims 45 to 58, wherein the first costimulatory signaling domain isolated from CD137.

60. The cell of any one of claims 45 to 59, wherein the primary signaling domain isolated from a polypeptide selected from the group consisting of: FcRγ, FcRβ, CD3γ, CD3δ, CD3ε, CD3ζ, CD22, CD79a, CD79b, and CD66d.

61. The cell of any one of claims 45 to 60, wherein the primary signaling domain isolated from CD3ζ.

62. The cell of any one of claims 45 to 61, wherein the second costimulatory signaling domain isolated from CD28.

63. The cell of any one of claims 45 to 62, wherein the anti-CD20 CCR comprises a CD8α hinge domain, a CD8α transmembrane domain, and a CD28 costimulatory signaling domain.

64. The cell of any one of claims 45 to 63, wherein the cell expresses an anti-CD79A CAR comprising an amino acid sequence set forth in any one of SEQ ID NOs: 17-20 and an anti-CD20 CCR comprising an amino acid sequence set forth in SEQ ID NO: 33 or SEQ ID NO: 35.

65. The cell of any one of claims 45 to 64, wherein the cell comprises a first polynucleotide encoding the anti-CD79A CAR and a second polynucleotide encoding the anti-CD20 CCR.

66. The cell of any one of claims 45 to 64, wherein an isolated polynucleotide encodes the anti-CD79A CAR and the anti-CD20 CCR.

67. The cell of claim 66, wherein the isolated polynucleotide encodes the anti-CD79A CAR, an IRES sequence, and the anti-CD20 CCR.

68. The cell of claim 66, wherein the isolated polynucleotide encodes the anti-CD79A CAR, a polypeptide cleavage signal, and the anti-CD20 CCR.

69. The cell of claim 68, wherein the polypeptide cleavage signal is a viral self-cleaving polypeptide.

70. The cell of claim 68 or claim 69, wherein the polypeptide cleavage signal is a viral self-cleaving 2A polypeptide.

71. The cell of any one of claims 68 to 70, wherein the polypeptide cleavage signal is a viral self-cleaving polypeptide selected from the group consisting of: a foot-and-mouth disease virus (FMDV) 2A (F2A) peptide, an equine rhinitis A virus (ERAV) 2A (E2A) peptide, a Thosea asigna virus (TaV) 2A (T2A) peptide, a porcine teschovirus-1 (PTV-1) 2A (P2A) peptide, a Theilovirus 2A peptide, and an encephalomyocarditis virus 2A peptide.

72. The cell of any one of claims 45 to 71, wherein the cell comprises insertion or deletion of one or more nucleotides in a homing endonuclease (HE) variant cleavage target site or a megaTAL cleavage target site in the casitas B-lineage (Cbl) lymphoma proto-oncogene B (CBLB) gene.

73. The cell of claim 72, wherein the HE variant introduces one or more insertions or deletions into the HE target site in the CBLB gene set forth in SEQ ID NO: 55.

74. The cell of claim 72, wherein the megaTAL introduces one or more insertions or deletions into the megaTAL target site in the CBLB gene set forth in SEQ ID NO: 56.

75. The cell of any one of claims 72 to 74, wherein the insertions or deletions in the CBLB gene decrease CBLB expression, function, and/or activity.

76. The cell of any one of claims 45 to 75, wherein the cell comprises one or more modified CBLB alleles.

77. The cell of any one of claims 45 to 76, wherein the cell comprises one or more modified CBLB alleles that do not express or produce CBLB or that express or produce non-functional CBLB.

78. The cell of any one of claims 45 to 71, wherein the cell comprises insertion or deletion of one or more nucleotides in a homing endonuclease (HE) variant cleavage target site or a megaTAL cleavage target site in the programmed cell death 1 (PDCD-1) gene or.

79. The cell of claim 78, wherein the HE variant introduces one or more insertions or deletions into the HE target site in the PDCD-1 gene set forth in SEQ ID NO: 51.

80. The cell of claim 78, wherein the megaTAL introduces one or more insertions or deletions into the megaTAL target site in the PDCD-1 gene set forth in SEQ ID NO: 52.

81. The cell of any one of claims 78 to 80, wherein the insertions or deletions in the PDCD-1 gene decrease PDCD-1 expression, function, and/or activity.

82. The cell of any one of claims 45 to 71, wherein the cell comprises one or more modified PDCD-1 alleles.

83. The cell of any one of claims 45 to 71, wherein the cell comprises one or more modified PDCD-1 alleles that do not express or produce PDCD-1 or that express or produce non-functional PDCD-1.

84. The cell of any one of claims 45 to 83, wherein the cell is a hematopoietic cell.

85. The cell of any one of claims 45 to 84, wherein the cell is a hematopoietic stem or progenitor cell.

86. The cell of any one of claims 45 to 85, wherein the cell is a CD34+ hematopoietic stem or progenitor cell.

87. The cell of any one of claims 45 to 83, wherein the cell is an immune effector cell.

88. The cell of any one of claims 45 to 83, wherein the cell is a T cell.

89. The cell of any one of claims 45 to 83, wherein the cell is a CD3⁺, CD4⁺, and/or CD8⁺ cell.

90. The cell of any one of claims 45 to 83, wherein the cell is a cytotoxic T lymphocytes (CTLs), a tumor infiltrating lymphocytes (TILs), or a helper T cell.

91. The cell of any one of claims 45 to 83, wherein the cell is a natural killer (NK) cell or natural killer T (NKT) cell.

92. A population of cells comprising a plurality of cells of any one of claims 45 to 91.

93. A population of cells comprising one or more hematopoietic stem or progenitor cells of claim 85 and one or more immune effector cells of claim 87.

94. A population of cells comprising one or more CD34+ hematopoietic stem or progenitor cells of claim 86 and one or more T cells of claim 88.

95. A composition comprising the cells of any one of claims 45 to 91 and a physiologically acceptable excipient.

96. A composition comprising the population of cells of any one of claims 92 to 94 and a physiologically acceptable excipient.

97. A method for killing cancer cells that express CD79A or CD20 in a subject, comprising administering to the subject therapeutically effective amount of the composition of claim 95 or claim 96.

98. A method for killing cancer cells that express CD79A and CD20 in a subject, comprising administering to the subject therapeutically effective amount of the composition of claim 95 or claim 96.

99. A method for killing cancer cells that express CD79A and/or CD20 in a subject, comprising administering to the subject therapeutically effective amount of the composition of claim 95 or claim 96.

100. A method for decreasing the number of cancer cells that express CD79A or CD20 in a subject, comprising administering to the subject therapeutically effective amount of the composition of claim 95 or claim 6 sufficient to decrease the number of cancer cells that express CD79A or CD20 compared to the number of the cancer cells that express CD79A or CD20 prior to the administration.

101. A method for decreasing the number of cancer cells that express CD79A and CD20 in a subject, comprising administering to the subject therapeutically effective amount of the composition of claim 95 or claim 96 sufficient to decrease the number of cancer cells that express CD79A and CD20 compared to the number of the cancer cells that express CD79A and CD20 prior to the administration.

102. A method for decreasing the number of cancer cells that express CD79A and/or CD20 in a subject, comprising administering to the subject therapeutically effective amount of the composition of claim 95 or claim 96 sufficient to decrease the number of cancer cells that express CD79A and/or CD20 compared to the number of the cancer cells that express CD79A and/or CD20 prior to the administration.

103. A method of treating a cancer in a subject in need thereof, comprising administering to the subject a therapeutically effect amount of the composition of claim 95 or claim 96.

104. The method of claim 103, wherein the cancer is a solid cancer.

105. The method of claim 104, wherein the solid cancer is an osteosarcoma or Ewing's sarcoma.

106. The method of claim 103, wherein the cancer is a liquid cancer.

107. The method of claim 106, wherein the liquid cancer is a hematological malignancy.

108. The method of claim 106 or claim 107, wherein the cancer is non-Hodgkin's lymphoma, acute lymphocytic leukemia (ALL), chronic lymphocytic leukemia (CLL), hairy cell leukemia (HCL), multiple myeloma (MM), acute myeloid leukemia (AML), or chronic myeloid leukemia (CML).

109. The method of claim 108, wherein the non-Hodgkin's lymphoma is Burkitt's lymphoma, small lymphocytic lymphoma (SLL), diffuse large B cell lymphoma (DLBCL), follicular lymphoma (FL), mantle cell lymphoma (MCL), or marginal zone lymphoma (MZL).

110. The method of claim 108, wherein the non-Hodgkin's lymphoma is diffuse large B cell lymphoma (DLBCL).

111. The method of claim 106 or claim 107, wherein the cancer is a MM selected from the group consisting of: overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

112. A method for treating a subject that has DLBCL comprising administering to the subject a therapeutically effect amount of the composition of claim 95 or claim 96.

113. A method for ameliorating at one or more symptoms associated with a cancer expressing CD79A and/or CD20 in a subject, comprising administering to the subject a therapeutically effective amount of the composition of claim 95 or claim 96 sufficient to ameliorate at least one symptom associated with cancer cells that express CD79A and/or CD20.

114. The method of claim 113, wherein the one or more symptoms ameliorated are selected from the group consisting of: weakness, fatigue, shortness of breath, easy bruising and bleeding, frequent infections, enlarged lymph nodes, distended or painful abdomen, bone or joint pain, fractures, unplanned weight loss, poor appetite, night sweats, persistent mild fever, and decreased urination.

115. A method of generating a population of cells that expresses the fusion polypeptide of any one of claims 1 to 33 comprising introducing into the population of cells the polynucleotide of any one of claims 34 to 36, or the vector of any one of claims 37 to 42.

116. A method of generating a population of cells that expresses an anti-CD79A CAR and an anti-CD20 CCR comprising introducing into the population of cells one or more polynucleotides encoding the anti-CD79A CAR and the anti-CD20 CCR as set forth in any one of claims 1 to 33.

117. A method of generating a population of cells that expresses an anti-CD79A CAR and an anti-CD20 CCR comprising introducing into the population of cells a polynucleotide encoding the fusion polypeptide sequence set forth in any one of SEQ ID NOs: 1 to 33.

118. A method of generating a population of cells that expresses an anti-CD79A CAR and an anti-CD20 CCR comprising introducing into the population of cells a first polynucleotide encoding the anti-CD79A CAR set forth in any one of SEQ ID NOs: 17-20 and a second polynucleotide encoding the anti-CD20 CCR sequence set forth in SEQ ID NO: 33 or SEQ ID NO: 35.

119. A method of generating a population of cells that expresses an anti-CD79A CAR and an anti-CD20 CCR comprising introducing into the population of cells the polynucleotide sequence set forth in SEQ ID NO: 38 or SEQ ID NO: 40.

120. A method of generating a population of cells that expresses an anti-CD79A CAR and an anti-CD20 CCR comprising introducing into the population of cells a first polynucleotide sequence set forth in any one of SEQ ID NOs: 21 to 24 and a second polynucleotide sequence set forth in SEQ ID NO: 34 or SEQ ID NO: 36.

121. The method of any one of claims 116 to 120, wherein one or more cells in the population of cells comprises one or more insertions or deletions in the PDCD-1 gene at a polynucleotide sequence set forth in SEQ ID NO: 51 that decrease or eliminate PDCD-1 expression and/or function.

122. The method of claim 121, wherein a polynucleotide encoding an HE variant that binds and cleaves the polynucleotide sequence set forth in SEQ ID NO: 51 is introduced into the population of cells.

123. The method of any one of claims 116 to 120, wherein one or more cells in the population of cells comprises one or more insertions or deletions in the PDCD-1 gene at a polynucleotide sequence set forth in SEQ ID NO: 52 that decrease or eliminate PDCD-1 expression and/or function.

124. The method of claim 123, wherein a polynucleotide encoding a megaTAL that binds and cleaves the polynucleotide sequence set forth in SEQ ID NO: 52 is introduced into the population of cells.

125. The method of any one of claims 116 to 120, wherein one or more cells in the population of cells comprises one or more insertions or deletions in the CBLB gene at a polynucleotide sequence set forth in SEQ ID NO: 55 that decrease or eliminate CBLB expression and/or function.

126. The method of claim 125, wherein a polynucleotide encoding an HE variant that binds and cleaves the polynucleotide sequence set forth in SEQ ID NO: 55 is introduced into the population of cells.

127. The method of any one of claims 116 to 120, wherein one or more cells in the population of cells comprises one or more insertions or deletions in the CBLB gene at a polynucleotide sequence set forth in SEQ ID NO: 56 that decrease or eliminate CBLB expression and/or function.

128. The method of claim 127, wherein a polynucleotide encoding a megaTAL that binds and cleaves the polynucleotide sequence set forth in SEQ ID NO: 56 is introduced into the population of cells.

129. The method of any one of claims 115 to 128, wherein the population of cells comprises hematopoietic stem or progenitor cells.

130. The method of any one of claims 115 to 128, wherein the population of cells comprises CD34+ hematopoietic stem or progenitor cells.

131. The method of any one of claims 115 to 128, wherein the population of cells comprises immune effector cells.

132. The method of any one of claims 115 to 128, wherein the population of cells comprises T cells, NK cells, and/or NKT cells.

133. The method of any one of claims 115 to 128, wherein the population of cells comprises T cells.
